(19) United States
(12) Patent Application Publication
(10) Pub. No.: US 2003/0235577 A1
(43) Pub. Date: Dec. 25, 2003

(54) Methods and compositions for preventing and treating microbial infections

(76) Inventors: Steven D. Shapiro, Boston, MA (US); William O. Hartzell, Westborough, MA (US)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 10/409,643

(22) Filed: Apr. 8, 2003

Related U.S. Application Data
(60) Provisional application No. 60/370,649, filed on Apr. 8, 2002.

(51) Int. Cl. A61K 38/46; C12N 9/64

(52) U.S. Cl. 424/94.65; 435/226; 514/6

(57) ABSTRACT

The invention involves administration of MMPAP-12 polypeptides and nucleic acids for the treatment or prevention of infectious disease associated with microorganisms in subjects. The invention also relates to kits and compositions relating to the MMPAP-12 molecules.

### RELATED APPLICATIONS

This application claims priority under 35 U.S.C. §119 from U.S. provisional application serial number 60/370,649, filed Apr. 8, 2002.

### GOVERNMENT SUPPORT

This invention was made in part with government support under grant number RO1 HL55160 from the National Institutes of Health (NIH). The government may have certain rights in this invention.

### FIELD OF THE INVENTION

The present invention relates to the use of MMPAP-12 polypeptides and nucleic acids in the treatment of microbial disorders (e.g., bacterial infections, viral infections, fungal infections, parasitic infections, etc.).

### BACKGROUND OF THE INVENTION

Infectious disease is one of the leading causes of death throughout the world. In the United States alone the death rate due to infectious disease rose 58% between 1980 and 1992. During this time, the use of anti-infective therapies to combat infectious disease has grown significantly and is now a multi-billion dollar a year industry. Even with these increases in anti-infective agent use, the treatment and prevention of infectious disease remains a challenge to the medical community throughout the world. In general, there are three types of anti-infective agents, anti-bacterial agents, anti-viral agents, and anti-fungal agents, and even within these classes of agents there is some overlap with respect to the type of microorganism they are useful for treating.

Anti-bacterial agents kill or inhibit bacteria, and include antibiotics as well as other synthetic or natural compounds having similar functions. Antibiotics are low-molecular-weight molecules that are produced as secondary metabolites by cells, such as microorganisms. In general, antibiotics interfere with one or more bacterial functions or structures which are specific for the microorganism and which are not present in host cells.

One of the problems with anti-infective therapies is the side effects occurring in the host that is treated with the anti-infective. For instance, many anti-infectious agents can kill or inhibit a broad spectrum of microorganisms and are not specific for a particular type of species. Treatment with these types of anti-infectious agents results in the killing of the normal microbial flora living in the host, as well as the infectious microorganism. The loss of the microbial flora can lead to disease complications and predispose the host to infection by other pathogens, since the microbial flora compete with and function as barriers to infectious pathogens. Other side effects may arise as a result of specific or non-specific effects of these chemical entities on non-microbial cells or tissues of the host.

Another problem with wide-spread use of anti-infectants is the development of antibiotic resistant strains of microorganisms. Already, vancomycin-resistant enterococci, penicillin-resistant pneumococci, multi-resistant S. aureus, and multi-resistant tuberculosis strains have developed and are becoming major clinical problems. Widespread use of anti-infectants will likely produce many antibiotic-resistant strains of bacteria. As a result, new anti-infective strategies will be required to combat these microorganisms.

### SUMMARY OF THE INVENTION

Improved methods and products for the prevention and/or treatment of microbial disorders (e.g., bacterial infections, viral infections, fungal infections, parasitic infections, etc.).

According to one aspect of the invention methods for treating or preventing an infection in a subject having or at risk of developing the infection are provided. The methods include administering to a subject in need of such treatment a therapeutically effective amount of an MMPAP-12 polypeptide molecule, or functional homolog thereof for treating or preventing the infection. In some embodiments, the MMPAP-12 polypeptide molecule is selected from the group consisting of SEQ ID NOs:1-6, 36, 37, 42, and 43. In certain embodiments, the infection is a bacterial infection. In some embodiments, the subject is a vertebrate. In certain embodiments, the subject is human. In some embodiments, the polypeptide molecule is administered systemically. In certain embodiments, the polypeptide molecule is administered topically.

According to another aspect of the invention, methods for treating or preventing an infection in a subject having or at risk of developing the infection are provided. The methods include administering to a subject in need of such treatment a therapeutically effective amount of an MMPAP-12 nucleic acid molecule, or functional homolog thereof, for treating or preventing the infection. In some embodiments, the MMPAP-12 nucleic acid molecule is selected from the group consisting of SEQ ID NOs:7-12, 38, 39, 44, and 45. In certain embodiments, the infection is a bacterial infection. In some embodiments, the subject is a vertebrate. In certain embodiments, the subject is human. In some embodiments, the polypeptide molecule is administered systemically. In certain embodiments, the polypeptide molecule is administered topically.

According to yet another aspect of the invention, isolated MMPAP-12 polypeptide molecules are provided. The isolated MMPAP-12 polypeptide molecules, do not have an amino acid sequence set forth as SEQ ID NO:13 or SEQ ID NO:15. In some embodiments, the polypeptide molecule is selected from the group consisting of SEQ ID NOs:1-6, 36, 37, 42, and 43, and functional homologs thereof.

According to another aspect of the invention, therapeutic compositions are provided. The therapeutic compositions include the foregoing isolated MMPAP-12 polypeptide molecule in a pharmaceutically acceptable carrier.

According to another aspect of the invention, an isolated nucleic acid molecule that encodes the any of the foregoing isolated polypeptides is provided. The isolated nucleic acid molecule does not have a nucleotide sequence selected from the group consisting of SEQ ID NO:14 and SEQ ID NO:16.

According to yet another aspect of the invention, therapeutic compositions are provided. The compositions include any of the foregoing isolated nucleic acid molecules, in a pharmaceutically acceptable carrier.

According to another aspects of the invention, expression vectors are provided. The expression vectors include any of the foregoing isolated nucleic acid molecules operably linked to a promoter.

According to another aspect of the invention, host cell transformed or transfected with the foregoing expression vectors are provided.

According to another aspect of the invention, transgenic non-human animals that include any of the foregoing expression vectors are provided.

According to another aspect of the invention, transgenic non-human animals that express a variable level of an MMPAP-12 molecule are provided.

According to another aspect of the invention, methods for producing an MMPAP-12 polypeptide molecule are provided. The methods include providing an isolated MMPAP-12 nucleic acid molecule operably linked to a promoter, wherein the MMPAP-12 nucleic acid molecule encodes the MMPAP-12 polypeptide molecule or a fragment thereof, and expressing the MMPAP-12 nucleic acid molecule in an expression system. In some embodiments, the method also includes isolating the MMPAP-12 polypeptide or fragment thereof from the expression system. In certain embodiments, the MMPAP-12 nucleic acid molecule is selected from the group consisting of SEQ ID NOs:7-12, 38, 39, 44, and 45.

According to another aspect of the invention, kits are provided. The kits include at least one container housing any of the foregoing isolated MMPAP-12 polypeptide molecules, and instructions for administration of the polypeptide. In some embodiments, the MMPAP-12 polypeptide molecule , includes an amino acid sequence selected from the group consisting of SEQ ID NOs. 1-6, 36, 37, 42, and 43.

According to another aspect of the invention, kits are provided. The kits include at least one container housing any of the foregoing MMPAP-12 nucleic acid molecules, and instructions for administration of the nucleic acid. In some embodiments, the MMPAP-12 nucleic acid molecule includes a nucleotide sequence selected from the group consisting of SEQ ID NOs:7-12, 38, 39, 44, and 45.

According to another aspect of the invention, anti-microbial compositions are provided. The anti-microbial compositions include the polypeptide of claim C1 in contact with a surface of a material or mixed with a suitable material. In some embodiments, the material is selected from the group consisting of: food, liquid, an instrument, a bead, a film, a monofilament, an unwoven fabric, sponge, cloth, a knitted fabric, a short fiber, a tube, a hollow fiber, an artificial organ, a catheter, a suture, a membrane, a bandage, and gauze. In certain embodiments, the anti-microbial is an anti-bacterial.

According to another aspect of the invention, methods of preventing or treating microbial contamination of a material are provided. The methods include contacting the material with an MMPAP-12 polypeptide in an effective amount to prevent or reduce the level of microbial contamination of the material. In some embodiments, the MMPAP-12 polypeptide includes an amino acid sequence selected from the group consisting of SEQ ID NOs:1-6, 36, 37, 42, and 43, and functional homologs thereof. In certain embodiments, the microbial contamination is bacterial contamination. In some embodiments, the material is aqueous. In certain embodiments, the material is drinking water. In some embodiments, the material comprises blood, a body effusion, tissue, or cell. In some embodiments, the material is food.

According to another aspect of the invention, methods for preparing an animal model of a disorder characterized by aberrant expression of an MMPAP-12 molecule are provided. The methods include administering to a non-human subject an effective amount of an antisense, siRNA, or RNAi molecule to an MMPAP-12 nucleic acid molecule to reduce expression of the MMPAP-12 nucleic acid molecule in the non-human subject.

According to another aspect of the invention, methods for preparing a non-human animal model of a disorder characterized by aberrant expression of an MMPAP-12 molecule are provided. The methods include administering to a non-human subject an effective amount of a binding polypeptide to an MMPAP-12 polypeptide to reduce expression of the MMPAP-12 polypeptide in the non-human subject. In some embodiments, the binding polypeptide is an antibody or an antigen-binding fragment thereof. In certain embodiments, the antibodies or antigen-binding fragments are labeled with one or more cytotoxic agents

According to another aspect of the invention, antisense, (RNAi and/or siRNA molecules are provided. The antisense molecules include a sequence that binds with high stringency to an MMPAP-12 nucleic acid but does not bind to a nucleic acid that encodes a protease domain of an MMP-12 nucleic acid. In some embodiments, the antisense binds to an MMPAP-12 nucleic acid selected from the group consisting of SEQ ID NOs:7-12, 38, 39, 44, and 45.

According to another aspect of the invention, kits for preparing a non-human animal model of a MMPAP-12-associated disorder in a subject are provided. The kits include one or more of the foregoing antisense molecules, and instructions for the use of the antisense molecule in the preparation of a non-human animal model of a disorder associated with aberrant expression of an MMPAP-12 molecule

Each of the limitations of the invention can encompass various embodiments of the invention. It is, therefore, anticipated that each of the limitations of the invention involving any one element or combinations of elements can be included in each aspect of the invention.

### BRIEF DESCRIPTION OF THE DRAWINGS

Figures are not required for enabling the claimed invention.

FIG. 1 is a diagram of the metalloproteinase domain structure. MMPs share common features including a proenzyme domain (I), a catalytic domain (II), and a C-terminal domain (III), which is thought to define substrate specificity. The catalytic Zn interacts with a conserved cysteine (C) in domain I to maintain the proenzyme in an inactive conformation. Matrilysin lacks domain III, and the gelatinases have an additional domain similar to the fibronectin type II domain (Gelatin-binding), which interrupts the catalytic domain and 92 kDa gelatinase has a region with homology to type V collagen.

FIG. 2. is a graph demonstrating the role of MMP-12 in post bone marrow survival and is a survival curve for MMP-12−/− and MMP-12+/+ mice after BMT.

FIG. 3. provides graphs of survival curves for MMP-12−/− and MMP-12+/+ mice during bacterial infections. FIG. 3A shows survival curve 72 hours after intraperitoneal inoculation with E. coli (K1) (1×10⁸ CFU). FIG. 3B shows 72 hour survival curve after peritoneal inoculation with S. aureus (4×10⁸ CFU). FIG. 3C shows a two week survival curve after intratracheal injection with S. aureus (3×10⁸ CFU). FIG. 3D shows a two week survival curve after hematogenous injection with (4×10⁸ CFU).

FIG. 4. consists of histograms of clearance of S. aureus from the lungs of MMP12−/− and MMP-12 mice. FIG. 4A shows the bacterial burden in lungs of MMP-12−/− and MMP-12+/+ at 2 and 24 hours after hematogenous injection. FIG. 4B shows bacterial load in lungs 2 hours after intratracheal inoculation with S. aureus (1×10⁶ CFU). FIGS. 4C and D are digitized photomicrographic images of histology from the lungs of mice stained with bacterial stain. FIG. 4E shows results indicating that MMP-12−/− alveolar macrophage contained intracellular S. aureus while MMP-12+/+ macrophage infrequently contained bacteria.

FIG. 5 is a histogram and digitized photomicrographic images demonstrating intracellular antimicrobial activity of MMP-12−/− and MMP-12+/+ macrophages against S. aureus. FIG. 5A shows results of an antibiotic protection assay for macrophages with intracellular bacterial load over 90 minute time course. Electron microscopy of macrophages S. aureus co-culture after 2 hours. FIG. 5B shows a digitized image of a micrograph of MMP-12+/+ macrophage with bacteria sequestered in phagosome. FIG. 5C is a digitized image of a micrograph showing MMP-12−/− macrophage after co-incubation with large intracellular bacterial proliferation.

FIG. 6 provides bar graphs of results when functional full-length recombinant human MMP-12 was incubated with S. aureus in a 5% LB culture. FIG. 6A shows results of a dose response curve showed that MMP-12 had 90% bacterial kill at 16 μg/ml after 2-hour incubation. FIG. 6B shows results when recombinant c-terminal domain co-incubated with S. aureus, which showed similar activity and dose response as the full length MMP-12 with a 90% antimicrobial activity at 20 μg/ml.

FIG. 7 is a graph that illustrates the antimicrobial activity of MMPAP-12 C-terminal fragment. S. aureus was co-incubated with the MMP-12 c-terminal and a hydrophilic fluorescent dye was added. The results indicated that MMP-12 carboxy terminal has bactericidal activity by disrupting bacterial cell membrane against S. aureus.

FIG. 8 provides graphs of results of additional trials were performed as described with (FIG. 8A) 60 mice for S. aureus peritonitis and (FIG. 8B) 11 mice for E. coli (K1) peritonitis. The results indicate that the MMP-12+/+ mice had a lower mortality rate than their MMP-12−/− counterparts.

FIG. 9 provides a list conserved regions of MMP-12 C-terminal homology of members of the MMP family. The sequences are: rabbit: DRHQVFLFKGDKFWLISHL (SEQ ID NO: 46); Rat: GRNQLFLFKDEKYWLINNL (SEQ ID NO;47); Mouse; SRNQLFLFKDEKYWLINNL (SEQ ID NO:48); and Human: ARNQVFLFKDDKYWLISNL (SEQ ID NO:49). A list of murine MMP C-terminal homology is also provided. The sequences are: MMP-12: SRNQLFLFKDEKYWLINNL (SEQ ID NO:48); MMP-13: SRDLMFIFRGRKFWALNG (SEQ ID NO:50); MMP-8: DRDLVFLFKGRQYWALSG (SEQ ID NO:51); MMP-10: IFKGSQFWAVRGNEVQAG (SEQ ID NO:52); MMP-9: GALHFFKDGWYWKFLNH (SEQ ID NO:53); and MMP-2: FAGNEYWVYSASTLERGY (SEQ ID NO:54). FIG. 9B illustrates results of a propidium iodide exclusion assay our results, which revealed bacteria incubated in the presence of MMP-12 peptide had clumping and increased uptake of membrane impermeant dye compared to bacteria incubated with MMP-13 which had little dye uptake.

FIG. 10 provides a bar graph and digitized images of the effect of the MMP 12 C-terminal fragment (SEQ ID NO:37) on cell death. FIG. 10A shows a the number of bacterial cells plotted against the amount of the MMP-12 C-terminal fragment with which the cells were incubated. The graph indicates results for E. coli and S aureus. FIGS. 10 B and C show digitized images of the propidium iodide exclusion assay of our results, which revealed bacteria incubated in the presence of MMP-12 C-terminal peptide had clumping and increased uptake of membrane impermeant dye.

FIG. 11 is a bar graph of results from a dose response experiment in which samples of S aureus were incubated with various concentrations of murine peptide (SEQ ID NO: 37), human peptide (SEQ ID NO: 36) and Human SNP (SEQ ID NO:55). The amount of bacteria remaining at various the various times was determined for each group.

### DETAILED DESCRIPTION OF THE INVENTION

Matrix metalloproteinase-12 (MMP-12) is a member of the family of matrix degrading enzymes, a family of proteinases that are capable of degrading most extracellular matrix proteins. Due to its degradative capabilities, MMP-12 has been hypothesized to contribute to matrix destruction in disease states such as emphysema and aortic aneurysm formation. We present data that sheds new understanding on this matrix metalloproteinase as a component in host defense. We have identified a new and novel physiological function for MMP-12 as an antimicrobial agent. Surprisingly, at a protein, cellular, in vitro, and in vivo level, MMP-12 has antimicrobial properties. This novel non-enzymatic anti-microbial activity of MMP-12, functions systemically and intracellularly. In addition, we have identified novel fragments of MMP-12 that have antimicrobial properties. As used herein, the terms “microbial” and “antimicrobial” are used interchangeably with the terms “microorganism” and antimicroorganism” respectively.

The invention in part, relates to methods and products for the treatment of infectious disease using the MMP-12 polypeptides and their encoding nucleic acids as described herein. In addition, the invention also relates in some aspects to the use of these polypeptides, and the nucleic acids that encode the polypeptides, in compositions and methods directed to the prevention and treatment of infectious disease. As used herein the term “MMPAP-12 molecules” includes MMPAP-12 polypeptides and MMPAP-12 nucleic acids that encode the MMPAP-12 polypeptides. The MMPAP-12 molecules of the invention include human, mouse, rat, and rabbit polypeptides and nucleic acids. The MMPAP-12 polypeptides include fragments (i.e. pieces) of an MMP-12 polypeptide. These fragments are shorter than the full-length MMP-12 molecule.

The MMPAP-12 polypeptides, which are also referred to herein as MMP-12 fragments, of the invention can be screened for antimicrobial activity using the same type of assays as described herein (e.g. in the Examples section). Using such assays, the MMPAP-12 polypeptides that have the best antimicrobial activity can be identified. It is understood that any mechanism of action described herein for the MMP-12 fragments or MMPAP-12 polypeptides is not intended to be limiting, and the scope of the invention is not bound by any such mechanistic descriptions provided herein.

The human MMPAP-12 polypeptides of the invention include sequences that contain the amino acid sequence EARNQVFLFKDDKYWLISNLR (SEQ ID NO: 3) and 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, or 111 additional amino acids at its C-terminal end, wherein the amino acids that are added are identical to the corresponding amino acid in that position in the full-length human MMP-12 amino acid sequence (Genbank accession number NP_{—}002417, SEQ ID NO:13). For example, the human MMPAP-12 polypeptide that has five additional amino acids at the C-terminal end will have the amino acid sequence: EARNQVFLFKDDKYWLISNLRPEPNY (SEQ ID NO: 22), and the human MMPAP-12 polypeptide that has eight additional amino acids at the C-terminal end will have the amino acid sequence: EARNQVFLFKDDKYWLISNLRPEPNYPDSIH (SEQ ID NO:23).

The human MMPAP-12 polypeptides of the invention also include sequences that include the amino acid sequence EARNQVFLFKDDKYWLISNLR (SEQ ID NO:3) and have 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, or 338 additional amino acids at its N-terminal end, wherein the amino acids that are added are identical to the corresponding amino acid in that position in the full-length human MMP-12 sequence (Genbank Accession number NP_{—}002417, SEQ ID NO:13). For example, the human MMPAP-12 polypeptide that has five additional amino acids at the N-terminal end will have the amino acid sequence: AAYEIEARNQVFLFKDDKYWLISNLR (SEQ ID NO:24), and the human MMPAP-12 polypeptide that has twelve additional amino acids at the N-terminal end will have the amino acid sequence: TLPSGIEAAYEIEARNQVFLFKDDKYWLISNLR (SEQ ID NO:25).

The human MMPAP-12 polypeptides of the invention also include sequences that include EARNQVFLFKDDKYWLISNLR (SEQ ID NO:3) and have 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, or 111 additional amino acids at its C-terminal end and have 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, or 338 additional amino acids at its N-terminal end, wherein the amino acids that are added will be identical to the amino acid in that position in the full-length human MMP-12 sequence (Genbank Accession number NP_{—}002417, SEQ ID NO:13). The human MMPAP12 polypeptides of the invention do not include the full-length human MMP-12 sequence. For example, the human MMPAP-12 polypeptide that has five additional amino acids at the N-terminal end and five additional amino acids at its C-terminal end will have the amino acid sequence: AAYEIEARNQVFLFKDDKYWLISNLRPEPNY (SEQ ID NO:26), and the human MMPAP-12 polypeptide that has 12 additional amino acids at the N-terminal end and five additional amino acids at its C-terminal end, will have the amino acid sequence: TLPSGIEAAYEIEARNQVFLFKDDKYWLISNLRPEPNY (SEQ ID NO: 27). Yet another human MMPAP-12 polypeptide of the invention is the amino acid sequence EARNQVFLFKDDKYWLISNLRP (SEQ ID NO:42). The human MMPAP12 polypeptides of the invention do not include the full-length human MMP-12 sequence.

The human MMPAP-12 polypeptides of the invention also include sequences that are smaller than the amino acid sequence EARNQVFLFKDDKYWLISNLR (SEQ ID NO:3) and it will be understood that the sequence can be reduced in size by 1, 2, 3, 4, 5, 6, 7, 8, 9, or 10 amino acids from either or both termini, provided that the remaining sequence is at least about 10 amino acids in length/For example, the human MMPAP-12 polypeptides of the invention include the sequence that contains the amino acid sequence ARNQVFLFKDDKYWLISNLR (SEQ ID NO:36).

The mouse MMPAP-12 polypeptides of the invention include sequences that contain the amino acid sequence ESRNQLFLFKDEKYWLINNLV (SEQ ID NO: 6) and 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, or 110 additional amino acids at its C-terminal end, wherein the amino acids that are added are identical to the corresponding amino acid in that position in the full-length mouse MMP-12 amino acid sequence (Genbank accession number NP_{—}032631, SEQ ID NO:15). For example, the mouse MMPAP-12 polypeptide that has five additional amino acids at the C-terminal end will have the amino acid sequence: ESRNQLFLFKDEKYWLINNLVPEPHY (SEQ ID NO: 28), and the mouse MMPAP-12 polypeptide that has eight additional amino acids at the C-terminal end will have the amino acid sequence: ESRNQLFLFKDEKYWLINNLVPEPHYPRS (SEQ ID NO:29).

The mouse MMPAP-12 polypeptides of the invention also include sequences that include the amino acid sequence ESRNQLFLFKDEKYWLNNLV (SEQ ID NO:6) and have 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, or 331 additional amino acids at its N-terminal end, wherein the amino acids that are added are identical to the corresponding amino acid in that position in the full-length mouse MMP-12 sequence (Genbank Accession number NP_{—}032631, SEQ ID NO:15). For example, the mouse MMPAP-12 polypeptide that has five additional amino acids at the N-terminal end will have the amino acid sequence: AAYEIESRNQLFLFKDEKYWLINNLV (SEQ ID NO:30), and the human MMPAP-12 polypeptide that has twelve additional amino acids at the N-terminal end will have the amino acid sequence: SIPSAIQAAYEIESRNQLFLFKDEKYWLINNLV (SEQ ID NO:31).

The mouse MMPAP-12 polypeptides of the invention also include sequences that includes the amino acid sequence ESRNQLFLFKDEKYWLTNNLV (SEQ ID NO:6) and have 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, or 110 additional amino acids at its C-terminal end and have 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, or 331 additional amino acids at its N-terminal end, wherein the amino acids that are added will be identical to the amino acid in that position in the full-length mouse MMP-12 sequence (Genbank Accession number NP_{—}032631, SEQ ID NO:15). For example, the mouse MMPAP-12 polypeptide that has five additional amino acids at the N-terminal end and five additional amino acids at its C-terminal end will have the amino acid sequence: AAYEIESRNQLFLFKDEKYWLINNLVPEPHY (SEQ ID NO:32), and the mouse MMPAP-12 polypeptide that has 12 additional amino acids at the N-terminal end and five additional amino acids at its C-terminal end, will have the amino acid sequence: SIPSAIQAAYEIESRNQLFLFKDEKYWLINNLVPEPHY (SEQ ID NO: 33). Yet another mouse MMPAP-12 polypeptide of the invention is the amino acid sequence ESRNQLFLFKDEKYWLINNLVP (SEQ ID NO:43). The mouse MMPAP12 polypeptides of the invention do not include the full-length human MMP-12 sequence.

The mouse MMPAP-12 polypeptides of the invention also include sequences that are smaller than ESRNQLFLFKDEKYWLINNLV (SEQ ID NO:6) and it will be understood that the sequence can be reduced in size by 1, 2, 3, 4, 5, 6, 7, 8, 9, or 10 amino acids from either or both termini, provided that the remaining sequence is at least about 10 amino acids in length. For example, the mouse MMPAP-12 polypeptides of the invention include the sequence that contains the amino acid sequence SRNQLFLFKDEKYWLINNLV (SEQ ID NO:37).

The MMPAP-12 nucleic acids of the invention are those nucleic acids that encode the MMPAP-12 polypeptides of the invention as described herein. The amino acid sequences identified herein as MMPAP-12 polypeptides, and the nucleotide sequences encoding them, are sequences deposited in databases such as GenBank. The human MMPAP-12 polypeptide molecules disclosed herein set forth as SEQ ID NOs:1-3 and 36 are encoded by the human MMPAP-12 nucleic acids set forth as SEQ ID NOs:7-9 and 38 shown in Table 1. The mouse MMPAP-12 polypeptide molecules disclosed herein set forth as SEQ ID NOs:4-6 and 37 are encoded by the mouse MMPAP-12 nucleic acids set forth as SEQ ID NOs:10-12 and 39 shown in Table 1. The rat MMPAP-12 polypeptide molecules disclosed herein are set forth as SEQ ID NOs:17-19. The amino acid sequences of the full-length human, mouse, rat, and rabbit MMP-12 polypeptides are set forth as SEQ ID NO:13, 15, 17, and 21 respectively, which correspond to Genbank Accession Numbers: NP_{—}002417, NP_{—}032631, Q63341, and P79227 respectively. The nucleotide sequences of the full-length human, mouse MMP-12 nucleic acids are set forth as SEQ ID NO: 14 and 16, respectively, which correspond to Genbank Accession Numbers: NM_{—}002426, NM_{—}008605, respectively.

As used herein, the term “protease domain” of the human MMP-12 polypeptide means the amino acid positions 218-228 (inclusive) of the human MMP-12 polypeptide sequence published as Genbank Accession No: NP_{—}002417. As used herein, the term “protease domain” of the mouse MMP-12 polypeptide means the amino acid positions 211-221 (inclusive) of the mouse MMP-12 polypeptide sequence published as Genbank Accession No: NP_{—}032631. The nucleic acid protease domains of human and mouse are understood to be the nucleic acids that encode the above-referenced polypeptide protease domains respectively. The protease domain is also known as the zinc-binding domain.

TABLE 1
Sequence descriptions for MMPAP-12 and MMP-12 Polypeptides
and Nucleic acids and Primers

| | Amino Acid | |
|---|---|---|
| Sequence Name/description | SEQ ID NO | Nucleic Acid SEQ ID NO. |
| Human MMPAP-12 | 1 | 7 |
| Human MMPAP-12 | 2 | 8 |
| Human MMPAP-12 | 3 | 9 |
| Mouse MMPAP-12 | 4 | 10 |
| Mouse MMPAP-12 | 5 | 11 |
| Mouse MMPAP-12 | 6 | 12 |
| Human MMP-12 | 13 | 14 |
| Mouse MMP-12 | 15 | 16 |
| Rat MMP-12 | 17 | |
| Rat MMPAP-12 | 18 | |
| Rat MMPAP-12 | 19 | |
| Rat MMPAP-12 | 20 | |
| Rabbit MMP-12 | 21 | |
| Human MMPNP-12 | 22 | |
| Human MMPAP-12 | 23 | |
| Human MMPAP-12 | 24 | |
| Human MMPAP-12 | 25 | |
| Human MMPAP-12 | 26 | |
| Human MMPAP-12 | 27 | |
| Mouse MMPAP-12 | 28 | |
| Mouse MMPAP-12 | 29 | |
| Mouse MMPAP-12 | 30 | |
| Mouse MMPAP-12 | 31 | |
| Mouse MMPAP-12 | 32 | |
| Mouse MMPAP-12 | 33 | |
| 5′ Primer | 34 | |
| 3′ Primer | 35 | |
| Human MMPAP-12 | 36 | 38 |
| Mouse MMPAP-12 | 37 | 39 |
| Mouse MMPAP-12 | 40 | |
| Peptide I | | |
| Mouse MMPAP-12 | 41 | |
| Peptide II | | |
| Human MMPAP-12 | 42 | 44 |
| Mouse MMPAP-12 | 43 | 45 |
| Rabbit MMP-12 fragment | 46 | |
| Rat MMP-12 fragment | 47 | |
| Mouse MMP-12 fragment | 48 | |
| Human MMP-12 fragment | 49 | |
| Mouse MMP-13 fragment | 50 | |
| Mouse MMP-8 fragment | 51 | |
| Mouse MMP-10 fragment | 52 | |
| Mouse MMP-9 fragment | 53 | |
| Mouse MMP-2 fragment | 54 | |
| Human MMP-12 SNP | 55 | |

The discovery that these polypeptides have an antimicrobial activity is unexpected. The identification of these antimicrobial molecules of the invention provides a basis for methods of treating microbial infection, therapeutic pharmaceutical agents and compounds, and other uses and methods described herein. Thus, an aspect of the invention is those nucleic acid sequences that code for MMPAP-12 polypeptides and polypeptide fragments thereof, which do not necessarily have an antimicrobial activity.

The invention also includes in some aspects isolated MMPAP-12 polypeptides and fragments thereof encoded by the nucleic acid molecules of the invention. Such MMPAP-12 polypeptides are useful, for example, alone or as fusion proteins to generate antibodies, and as components of an immunoassay. MMPAP-12 polypeptides can be isolated from biological samples including tissue or cell homogenates. The term “isolated” as used herein refers to a molecular species that is substantially free of other proteins, lipids, carbohydrates or other materials with which it is naturally associated. One skilled in the art can purify polypeptides, using standard techniques for protein purification. The isolated polypeptide will often yield a single major band on a non-reducing polyacrylamide gel. In the case of partially glycosylated polypeptides or those that have several start codons, there may be several bands on a non-reducing polyacrylamide gel, but these will form a distinctive pattern for that polypeptide. The purity of the polypeptide can also be determined by amino-terminal amino acid sequence analysis.

In addition to obtaining MMPAP-12 polypeptides of the invention via isolation, the MMPAP-12 polypeptides can also be expressed recombinantly in a variety of prokaryotic and eukaryotic expression systems by constructing an expression vector appropriate to the expression system, introducing the expression vector into the expression system, and isolating the recombinantly expressed protein. Short polypeptides, such as MMPAP-12 fragments, also can be synthesized chemically using well-established methods of peptide synthesis.

Fragments of a polypeptide preferably retain a distinct functional capability of the polypeptide. Functional capabilities that can be retained in a fragment of a polypeptide include antimicrobial activity, interaction with other polypeptides or fragments thereof, and selective binding of nucleic acids or proteins. One important activity is the antimicrobial activity.

The skilled artisan will also realize that conservative amino acid substitutions may be made in MMPAP-12 polypeptides to provide functionally equivalent variants, or homologs of the foregoing polypeptides, i.e, the variants retain the functional capabilities of the MMPAP-12 polypeptides (e.g. antimicrobial activity). As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Exemplary functionally equivalent variants or homologs of the MMPAP-12 polypeptides include conservative amino acid substitutions of in the amino acid sequences of proteins disclosed herein. Conservative substitutions of amino acids include substitutions made amongst amino acids within the following groups: (a) M, I, L, V; (b) F, Y, W; (c) K, R, H; (d) A, G; (e) S, T; (f) Q, N; and(g) E, D.

For example, upon determining that a peptide is an MMPAP-12 polypeptide, one can make conservative amino acid substitutions to the amino acid sequence of the peptide, and determine whether the variant so made retains antimicrobial activity.

Conservative amino-acid substitutions in the amino acid sequence of MMPAP-12 polypeptides to produce functionally equivalent variants of MMPAP-12 polypeptides typically are made by alteration of a nucleic acid encoding a MMPAP-12 polypeptide. Such substitutions can be made by a variety of methods known to one of ordinary skill in the art. For example, amino acid substitutions may be made by PCR-directed mutation, site-directed mutagenesis according to the method of Kunkel (Kunkel, Proc. Nat. Acad. Sci. U.S.A. 82: 488-492, 1985), or by chemical synthesis of a gene encoding a MMPAP-12 polypeptide. Where amino acid substitutions are made to a small unique fragment of a MMPAP-12 polypeptide, the substitutions can be made by directly synthesizing the peptide. The activity of functionally equivalent fragments of MMPAP-12 polypeptides can be tested by cloning the gene encoding the altered MMPAP-12 polypeptide into an insect, bacterial, or mammalian expression vector, introducing the vector into an appropriate host cell, expressing the altered polypeptide, and testing for a functional capability of the MMPAP-12 polypeptides as disclosed herein. Peptides that are chemically synthesized can be tested directly for function, e.g., for antimicrobial activity (see Examples).

The invention as described herein has a number of uses, some of which are described elsewhere herein.

The MMPAP-12 polypeptides of the invention, including fragments thereof, can also be used to screen peptide libraries, including phage display libraries, to identify and select peptide binding partners of the MMPAP-12 polypeptides of the invention. Such molecules can be used, as described, for screening assays, for purification protocols, for interfering directly with the functioning of MMPAP-12 polypeptides (e.g. in knock-out cells or animals as described herein) and for other purposes that will be apparent to those of ordinary skill in the art. For example, isolated MMPAP-12 polypeptides can be attached to a substrate (e.g., chromatographic media, such as polystyrene beads, or a filter), and then a solution suspected of containing the binding partner may be applied to the substrate. If a binding partner that can interact with MMPAP-12 polypeptides is present in the solution, then it will bind to the substrate-bound MMPAP-12 polypeptide. The binding partner then may be isolated.

The invention, therefore, embraces polypeptide binding agents which, for example, can be antibodies or fragments of antibodies having the ability to selectively bind to MMPAP-12 polypeptides. Antibodies include polyclonal and monoclonal antibodies, prepared according to conventional methodology.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. See, e.g., U.S. Pat. No. 4,816,567, 5,225,539, 5,585,089, 5,693,762 and 5,859,205.

Fully human monoclonal antibodies also can be prepared by immunizing mice transgenic for large portions of human immunoglobulin heavy and light chain loci. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

Thus, as will be apparent to one of ordinary skill in the art, the present invention also provides for F(ab′)₂, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

Thus, the invention involves polypeptides of numerous size and type that bind specifically to MMPAP-12 polypeptides, and complexes of both MMPAP-12 polypeptides and their binding partners. These polypeptides may be derived also from sources other than antibody technology. For example, such polypeptide binding agents can be provided by degenerate peptide libraries which can be readily prepared in solution, in immobilized form or as phage display libraries. Combinatorial libraries also can be synthesized of peptides containing one or more amino acids. Libraries further can be synthesized of peptoids and non-peptide synthetic moieties.

Phage display can be particularly effective in identifying binding peptides useful according to the invention. Briefly, one prepares a phage library (using e.g. m13, fd, or lambda phage), displaying inserts from 4 to about 80 amino acid residues using conventional procedures. The inserts may represent, for example, a completely degenerate or biased array. One then can select phage-bearing inserts which bind to the MMPAP-12 polypeptide. This process can be repeated through several cycles of reselection of phage that bind to the MMPAP-12 polypeptide. Repeated rounds lead to enrichment of phage bearing particular sequences. DNA sequence analysis can be conducted to identify the sequences of the expressed polypeptides. The minimal linear portion of the sequence that binds to the MMPAP-12 polypeptide can be determined. One can repeat the procedure using a biased library containing inserts containing part or all of the minimal linear portion plus one or more additional degenerate residues upstream or downstream thereof. Yeast two-hybrid screening methods also may be used to identify polypeptides that bind to the MMPAP-12 polypeptides.

Optionally, an antibody can be linked to one or more detectable markers (as described herein), or cytotoxic agent. Detectable markers include, for example, radioactive or fluorescent markers. Cytotoxic agents include cytotoxic radionuclides, chemical toxins and protein toxins.

The cytotoxic radionuclide or radiotherapeutic isotope may be an alpha-emitting isotope such as ²²⁵Ac, ²¹¹At, ²¹²Bi, or ²¹³Bi. Alternatively, the cytotoxic radionuclide may be a beta-emitting isotope such as ¹⁸⁶Rh, ¹⁸⁸Rh, ⁹⁰y, ¹³¹I, or ⁶⁷Cu. Further, the cytotoxic radionuclide may emit Auger and low-energy electrons such as the isotopes ¹²⁵I, ¹²³I, or ⁷⁷Br.

Suitable chemical toxins or include members of the enediyne family of molecules, such as chalicheamicin and esperamicin. Chemical toxins can also be taken from the group consisting of methotrexate, doxorubicin, melphalan, chlorambucil, ARA-C, vindesine, mitomycin C, cis-platinum, etoposide, bleomycin and 5-fluorouaracil. Other chemotherapeutic agents are known to those skilled in the art.

The invention also relates, in part, to the use of homologs of the MMPAP-12 polypeptides of the invention. As used herein, a “homolog” to an MMPAP-12 polypeptide is a polypeptide from a human or other animal that has a high degree of structural similarity to the identified MMPAP-12 polypeptides. Identification of MMPAP-12 polypeptide homologs may be useful in therapeutic drug design or in the production of animal models.

The invention also relates, in some aspects, to homologs and alleles of the nucleic acids encoding MMPAP-12 polypeptides of the invention, which can be identified by conventional techniques. Identification of human and/or other organism homologs of MMPAP-12 nucleic acids will be familiar to those of skill in the art. In general, nucleic acid hybridization is a suitable method for identification of homologous sequences of another species (e.g., mouse, rabbit, rat, cow, sheep), which correspond to a known sequence. Standard nucleic acid hybridization procedures can be used to identify related nucleic acid sequences of selected percent identity. For example, one can construct a library of cDNAs reverse transcribed from the mRNA of a selected tissue (e.g., lung) and use the nucleic acids identified herein to screen the library for related nucleotide sequences. The screening preferably is performed using high-stringency hybridization conditions to identify those sequences that are closely related by sequence identity.

The term “high stringency” as used herein refers to parameters with which the art is familiar. Nucleic acid hybridization parameters may be found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. More specifically, high-stringency conditions, as used herein, refers, for example, to hybridization at 65° C. in hybridization buffer (3.5×SSC, 0.02% Ficoll, 0.02% polyvinyl pyrrolidone, 0.02% Bovine Serum Albumin, 2.5 mM NaH₂PO₄(pH7), 0.5% SDS, 2 mM EDTA). SSC is 0.15 M sodium chloride/0.015 M sodium citrate, pH7; SDS is sodium dodecyl sulphate; and EDTA is ethylenediaminetetracetic acid. After hybridization, the membrane upon which the DNA is transferred is washed, for example, in 2×SSC at room temperature and then at 0.1−0.5×SSC/0.1×SDS at temperatures up to 68° C.

There are other conditions, reagents, and so forth that can be used, which result in a similar degree of stringency. The skilled artisan will be familiar with such conditions, and thus they are not given here. It will be understood, however, that the skilled artisan will be able to manipulate the conditions in a manner to permit the clear identification of homologs and alleles of MMPAP-12 polypeptide nucleic acids of the invention (e.g., by using lower stringency conditions). The skilled artisan also is familiar with the methodology for screening cells and libraries for expression of such molecules, which then are routinely isolated, followed by isolation of the pertinent nucleic acid molecule and sequencing.

In general, homologs and alleles typically will share at least 80% nucleotide identity and/or at least 80% amino acid identity to the sequences of MMPAP-12 nucleic acids and polypeptides, respectively, in some instances will share at least 85% nucleotide identity and/or at least 90% amino acid identity to the sequences of MMPAP-12 nucleic acids and polypeptides, respectively, in some instances will share at least 90% nucleotide identity and/or at least 95% amino acid identity to the sequences of MMPAP-12 nucleic acids and polypeptides, respectively, in some instances will share at least 95% nucleotide identity and/or at least 97% amino acid identity, in other instances will share at least 97% nucleotide identity and/or at least 98% amino acid identity, in other instances will share at least 99% nucleotide identity and/or at least 99% amino acid identity, and in other instances will share at least 99.5% nucleotide identity and/or at least 99.5% amino acid identity. The identity can be calculated using various, publicly available software tools developed by NCBI (Bethesda, Md.) that can be obtained through the internet. Exemplary tools include the BLAST system available from the website of the National Center for Biotechnology Information (NCBI) at the National Institutes of Health. Pairwise and ClustalW alignments (BLOSUM30 matrix setting) as well as Kyte-Doolittle hydropathic analysis can be obtained using the MacVector sequence analysis software (Oxford Molecular Group). Watson-Crick complements of the foregoing nucleic acids also are embraced by the invention. In silico methods can also be used to identify related sequences.

In screening for MMPAP-12 genes, a Southern blot may be performed using the foregoing conditions, together with a detectably labeled probe (e.g. radioactive or chemiluminescent probes). After washing the membrane to which the DNA is finally transferred, the membrane can be placed against X-ray film or a phosphorimager to detect the radioactive or chemiluminescent signal. In screening for the expression of MMPAP-12 polypeptide nucleic acids, Northern blot hybridizations using the foregoing conditions can be performed on samples taken from cells or subjects suspected of expressing the MMPAP-1 molecules of the invention.

Amplification protocols such as polymerase chain reaction using primers that hybridize to the sequences presented also can be used for detection of the MMPAP-12 polypeptide genes or expression thereof. Identification of related sequences can also be achieved using polymerase chain reaction (PCR) including RT-PCR, RT-real-time PCR, and other amplification techniques suitable for cloning related nucleic acid sequences. Preferably, PCR primers are selected to amplify portions of a nucleic acid sequence believed to be conserved (e.g., a catalytic domain, a DNA-binding domain, etc.). Again, nucleic acids are preferably amplified from a tissue-specific library (e.g., lung).

The invention also includes degenerate nucleic acids that include alternative codons to those present in the native materials. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating MMPAP-12 polypeptide. Similarly, nucleotide sequence triplets which encode other amino acid residues include, but are not limited to: CCA, CCC, CCG, and CCT (proline codons); CGA, CGC, CGG, CGT, AGA, and AGG (arginine codons); ACA, ACC, ACG, and ACT (threonine codons); AAC and AAT (asparagine codons); and ATA, ATC, and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code.

The invention also provides modified nucleic acid molecules, which include additions, substitutions and deletions of one or more nucleotides (preferably 1-20 nucleotides). In preferred embodiments, these modified nucleic acid molecules and/or the polypeptides they encode retain at least one activity or function of the unmodified nucleic acid molecule and/or the polypeptides, such as antimicrobial activity, etc. In certain embodiments, the modified nucleic acid molecules encode modified polypeptides, preferably polypeptides having conservative amino acid substitutions as are described elsewhere herein. The modified nucleic acid molecules are structurally related to the unmodified nucleic acid molecules and in preferred embodiments are sufficiently structurally related to the unmodified nucleic acid molecules so that the modified and unmodified nucleic acid molecules hybridize under stringent conditions known to one of skill in the art.

For example, modified nucleic acid molecules that encode polypeptides having single amino acid changes can be prepared. Each of these nucleic acid molecules can have one, two or three, four, five, or six nucleotide substitutions exclusive of nucleotide changes corresponding to the degeneracy of the genetic code as described herein. Likewise, modified nucleic acid molecules that encode polypeptides having two amino acid changes can be prepared which have, e.g., 2-6 nucleotide changes. Numerous modified nucleic acid molecules like these will be readily envisioned by one of skill in the art, including for example, substitutions of nucleotides in codons encoding amino acids 2 and 3, 2 and 4, 2 and 5, 2 and 6, and so on. In the foregoing example, each combination of two amino acids is included in the set of modified nucleic acid molecules, as well as all nucleotide substitutions which code for the amino acid substitutions. Additional nucleic acid molecules that encode polypeptides having additional substitutions (i.e., 3 or more), additions or deletions (e.g., by introduction of a stop codon or a splice site(s)) also can be prepared and are embraced by the invention as readily envisioned by one of ordinary skill in the art. Any of the foregoing nucleic acids or polypeptides can be tested by routine experimentation for retention of activity or structural relation to the nucleic acids and/or polypeptides disclosed herein. As used herein, the term, “functional homolog” means a homolog as described herein, that retains the antimicrobial property of the MMPAP-12 polypeptide, or encodes an MMPAP-12 polypeptide that possesses the antimicrobial property.

The invention also provides nucleic acid molecules that encode fragments of MMPAP-12 polypeptides. Fragments, can be used as probes in Southern and Northern blot assays to identify such nucleic acids, or can be used in amplification assays such as those employing PCR, including, but not limited to RT-PCR and RT-real-time PCR. As known to those skilled in the art, large probes such as 200, 250, 300 or more nucleotides are preferred for certain uses such as Southern and Northern blots, while smaller fragments will be preferred for uses such as PCR. Fragments also can be used to produce fusion proteins for generating antibodies or determining binding of the polypeptide fragments, or for generating immunoassay components. Likewise, fragments can be employed to produce nonfused fragments of the MMPAP-12 polypeptides, useful, for example, in the preparation of antibodies, and in immunoassays.

The invention also permits the construction of MMPAP-12 polypeptide gene “knock-out” or “knock-in” cells and/or animals, providing materials for studying certain aspects of microbial infection and treatments by regulating the expression of MMPAP-12 polypeptides. For example, a knock-in mouse may be constructed and examined for clinical parameters of increased antimicrobial properties in a mouse with upregulated expression of an MMPAP-12 polypeptide. In addition, a MMPAP-12 polypeptide “knock-out” cell and/or animal can be constructed and used to study aspects of microbial infection. A knock-out cell or animal can be generated by administering antisense, RNAi and/or siRNA molecules to reduce expression of MMPAP-12 polypeptides of the invention in the subject. Knock-out cells or animal models can also be generated by administering an effective amount of a molecule, such as an antibody, that specifically binds to a MMPAP-12 polypeptide in a subject. Such antibodies may inhibit the function of the polypeptide, thereby reducing its antimicrobial function, or the antibodies may include a cytotoxic or radioactive label that kills cells upon binding to the polypeptides of the invention. Such cellular or animal model may also be useful for assessing treatment strategies for microbial infection.

The invention relates in some aspects to methods of administering MMPAP-12 molecules for preventing and/or treating microorganism infections in subjects. As used herein, the term “prevent”, “prevented”, or “preventing” and “treat”, “treated” or “treating” when used with respect to the prevention or treatment of an infectious disease refers to a prophylactic treatment which increases the resistance of a subject to a microorganism or, in other words, decreases the likelihood that the subject will develop an infectious disease to the microorganism, as well as to a treatment after the subject has been infected in order to fight the infectious disease, e.g., reduce or eliminate it altogether or prevent it from becoming worse.

The MMPAP-12 polypeptide and nucleic acid molecules of the invention are useful for treating or preventing infectious disease in a subject. As used herein, a “subject” shall mean a human or vertebrate mammal including but not limited to a dog, cat, horse, cow, pig, sheep, goat, or primate, e.g., monkey. Non-human vertebrates that exist in close quarters and which are allowed to intermingle as in the case of zoo, farm, and research animals are also embraced as subjects for the methods of the invention. In some embodiments, a “subject” shall mean a non-mammalian vertebrate, such as a bird or fish. In some embodiments, a “subject” shall mean an invertebrate, and in yet other embodiments, a “subject” shall mean a plant.

The MMPAP-12 polypeptides and nucleic acids are useful in some aspects of the invention as prophylactics for the treatment of a subject at risk of developing an infectious disease where the exposure of the subject to a microorganism or expected exposure to a microorganism is known or suspected. A “subject at risk” of developing an infectious disease as used herein is a subject who has any risk of exposure to a microorganism, e.g. someone who is in contact with an infected subject or who is travelling to a place where a particular microorganism is found. For instance, a subject at risk may be a subject who is planning to travel to an area where a particular microorganism is found or it may even be any subject living in an area where a microorganism has been identified. A subject at risk of developing an infection includes those subjects that have a general risk of exposure to a microorganism, e.g., staphylococcus, but that don't have the active disease during the treatment of the invention, as well as subjects that are considered to be at specific risk of developing an infectious disease because of medical or environmental factors, that expose them to a particular microorganism. A subject at risk also includes transplant patients, an example of which, although not intending to be limiting is a subject who has undergone or will undergo a bone marrow transplant.

In addition to the use of the MMPAP-12 polypeptides and nucleic acids for prophylactic treatment, the invention also encompasses the use of the molecules for the treatment of a subject having a microorganism infection. A “subject having a microbial infection” is a subject that has had contact with a microbial organism. Thus, the microbial organism has invaded the body of the subject. The word “invade” as used herein refers to contact by the microbial organism with the external surface of the subject, e.g., skin or mucosal membranes and/or refers to the penetration of the external surface of the subject by the microbial organism.

An “infectious disease” or “infection”, as used herein, refers to a disorder arising from the invasion of a host, superficially, locally, or systemically, by an infectious microorganism. Infectious microorganisms include bacteria, viruses, and fungi. Bacteria are unicellular organisms which multiply asexually by binary fission. They are classified and named based on their morphology, staining reactions, nutrition and metabolic requirements, antigenic structure, chemical composition, and genetic homology. Bacteria can be classified into three groups based on their morphological forms, spherical (coccus), straight-rod (bacillus) and curved or spiral rod (vibrio, campylobacter, spirillum, and spirochaete). Bacteria are also more commonly characterized based on their staining reactions into two classes of organisms, gram-positive and gram-negative. Gram refers to the method of staining which is commonly performed in microbiology labs. Gram-positive organisms retain the stain following the staining procedure and appear a deep violet color. Gram-negative organisms do not retain the stain but take up the counter-stain and thus appear pink.

Bacteria have two main structural components, a rigid cell wall and protoplast (material enclosed by the cell wall). The protoplast includes cytoplasm and genetic material. Surrounding the protoplast is the cytoplasmic membrane which includes some of the cell respiratory enzymes and is responsible for the permeability of bacteria and transport of many small molecular weight substances. The cell wall surrounding the cytoplasmic membrane and protoplast is composed of mucopeptides which include complex polymers of sugars cross-linked by peptide chains of amino acids. The wall is also composed of polysaccharides and teichoic acids.

Infectious bacteria include, but are not limited to, gram negative and gram positive bacteria. Gram positive bacteria include, but are not limited to Pastuerella species, Staphylococci species, and Streptococcus species. Gram negative bacteria include, but are not limited to, Escherichia coli, Pseudomonas species, and Salmonella species. Specific examples of infectious bacteria include but are not limited to: Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria nmeningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic species.), Streptococcus pneumoniae, pathogenic Campylobacter sp., Enterococcus sp., Haemophilus influenzae, Bacillus antracis, corynebacterium diphtheriae, corynebacterium sp., Erysipelothrix rhusiopathiae, Clostridium perfringers, Clostridium tetani, Enterobacter aerogenes, Citrobacter, Klebsiella pneumoniae, Pasturella multocida, Bacteroides sp., Fusobacterium nucleatum, Streptobacillus moniliformis, Treponema palladium, Treponema pertenue, Leptospira, Rickettsia, and Actinomyces israelli.

Examples of bacterial infections for which methods of the invention can be used, include, but are not limited to: pneumonia, peritonitis, blood-borne infections, skin infections, corneal ulcers, meningitis, and urinary tract infections.

Infectious bacteria of plants include but are not limited to: Pseudomonadaceae, Rhizobiaceae, Enterobacteriaceae, Corynebacteriaceae and Streptomycetaceae. Phytopathogenic bacteria include, but are not limited to members of the order Pseudomonas, e.g. Pseudomonas tomato, Pseudomonas lachrymans, Ps. morsprunorum, Ps. phaseolicola, Ps. syringae and those of the order Xanthomonas, e.g. Xanthomonas oryzae, Xanthomonas vesicatoria, Xanthomonas phaseoli and Xanthomonas campestris, as well as Erwinia and Corynebacterium.

Viruses are small infectious agents which contain a nucleic acid core and a protein coat, but are not independently living organisms. A virus cannot survive in the absence of a living cell within which it can replicate. Viruses enter specific living cells either by endocytosis or direct injection of DNA (phage) and multiply, causing disease. The multiplied virus can then be released and infect additional cells. Some viruses are DNA-containing viruses and other are RNA-containing viruses.

Once the virus enters the cell it can cause a variety of physiological effects. One effect is cell degeneration, in which the accumulation of virus within the cell causes the cell to die and break into pieces and release the virus. Another effect is cell fusion, in which infected cells fuse with neighboring cells to produce syncytia. Other types of virus cause cell proliferation which results in tumor formation.

Viruses include, but are not limited to, interoviruses (including, but not limited to, viruses that the family picornaviridae, such as polio virus, coxsackie virus, echo virus), rotaviruses, adenovirus, hepatitus. Specific examples of viruses that have been found in humans include but are not limited to: Retroviridae (e.g. human immunodeficiency viruses, such as HIV-1 (also referred to as HTLV-III, LAV or HTLV-III/LAV, or HIV-ITI; and other isolates, such as HIV-LP; Picornaviriclae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bunyaviridae (e.g. Hantaan viruses, bunya viruses, phleboviruses and Nairo viruses); Arena viridae (hemorrhagic fever viruses); Reoviridae (e.g. reoviruses, orbiviurses and rotaviruses); Birnaviriclae; Hepadnaviridae (Hepatitis B virus); Parvoviricda (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviriclae (e.g. African swine fever virus); and unclassified viruses (e.g. the etiological agents of spongiform encephalopathies, the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

In addition to viruses that infect human subjects causing human disorders, the invention is also useful for treating other non-human vertebrates. Non-human vertebrates are also capable of developing infections which can be prevented or treated with the MMPAP-12 molecules disclosed herein. For instance, in addition to the treatment of infectious human diseases, the methods of the invention are useful for treating or preventing infections of non-human animals.

Infectious virus of both human and non-human vertebrates, include retroviruses, RNA viruses and DNA viruses. This group of retroviruses includes both simple retroviruses and complex retroviruses. The simple retroviruses include the subgroups of B-type retroviruses, C-type retroviruses and D-type retroviruses. An example of a B-type retrovirus is mouse mammary tumor virus (MMTV). The C-type retroviruses include subgroups C-type group A (including Rous sarcoma virus (RSV), avian leukemia virus (ALV), and avian mycloblastosis virus (AMV)) and C-type group B (including murine leukemia virus (MLV), feline leukemia virus (FeLV), murine sarcoma virus (MSV), gibbon ape leukemia virus (GALV), spleen necrosis virus (SNV), reticuloendotheliosis virus (RV) and simian sarcoma virus (SSV)). The D-type retroviruses include Mason-Pfizer monkey virus (MPMV) and simian retrovirus type 1 (SRV-1). The complex retroviruses include the subgroups of lentiviruses, T-cell leukemia viruses and the foamy viruses. Lentiviruses include HIV-1, but also include HIV-2, SIV, Visna virus, feline immunodeficiency virus (FIV), and equine infectious anemia virus (EIAV). The T-cell leukemia viruses include HTLV-1, HTLV-II, simian T-cell leukemia virus (STLV), and bovine leukemia virus (BLV). The foamy viruses include human foamy virus (HFV), simian foamy virus (SFV) and bovine foamy virus (BFV).

Examples of other RNA viruses that are antigens in vertebrate animals include, but are not limited to, the following: members of the family Reoviridae, including the genus Orthoreovirus (multiple serotypes of both mammalian and avian retroviruses), the genus Orbivirus (Bluetongue virus, Eugenangee virus, Kemerovo virus, African horse sickness virus, and Colorado Tick Fever virus), the genus Rotavirus (human rotavirus, Nebraska calf diarrhea virus, murine rotavirus, simian rotavirus, bovine or ovine rotavirus, avian rotavirus); the family Picornaviridae, including the genus Enterovirus (poliovirus, Coxsackie virus A and B, enteric cytopathic human orphan (ECHO) viruses, hepatitis A virus, Simian enteroviruses, Murine encephalomyclitis (ME) viruses, Poliovirus muris, Bovine enteroviruses, Porcine enteroviruses, the genus Cardiovirus (Encephalomyocarditis virus (EMC), Mengovirus), the genus Rhinovirus (Human rhinoviruses including at least 113 subtypes; other rhinoviruses), the genus Apthovirus (Foot and Mouth disease (FMDV); the family Calciviridae, including Vesicular exanthema of swine virus, San Miguel sea lion virus, Feline picornavirus and Norwalk virus; the family Togaviridae, including the genus Alphavirus (Eastern equine encephalitis virus, Semliki forest virus, Sindbis virus, Chikungunya virus, O'Nyong-Nyong virus, Ross river virus, Venezuelan equine encephalitis virus, Western equine encephalitis virus), the genus Flavirus (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); the family Rhabdoviridae, including the genus Vesiculovirus (VSV), Chandipura virus, Flanders-Hart Park virus), the genus Lyssavirus (Rabies virus), fish Rhabdoviruses, and two probable Rhabdoviruses (Marburg virus and Ebola virus); the family Arenaviridae, including Lymphocytic choriomeningitis virus (LCM), Tacaribe virus complex, and Lassa virus; the family Coronoaviridae, including Infectious Bronchitis Virus (IBV), Mouse Hepatitis virus, Human enteric corona virus, and Feline infectious peritonitis (Feline coronavirus).

Illustrative DNA viruses that infect vertebrate animals include, but are not limited to: the family Poxviridae, including the genus Orthopoxvirus (Variola major, Variola minor, Monkey pox Vaccinia, Cowpox, Buffalopox, Rabbitpox, Ectromelia), the genus Leporipoxvirus (Myxoma, Fibroma), the genus Avipoxvirus (Fowlpox, other avian poxvirus), the genus Capripoxvirus (sheeppox, goatpox), the genus Suipoxvirus (Swinepox), the genus Parapoxvirus (contagious postular dermatitis virus, pseudocowpox, bovine papular stomatitis virus); the family Iridoviridae (African swine fever virus, Frog viruses 2 and 3, Lymphocystis virus of fish); the family Herpesviridae, including the alpha-Herpesviruses (Herpes Simplex Types 1 and 2, Varicella-Zoster, Equine abortion virus, Equine herpes virus 2 and 3, pseudorabies virus, infectious bovine keratoconjunctivitis virus, infectious bovine rhinotracheitis virus, feline rhinotracheitis virus, infectious laryngotracheitis virus) the Beta-herpesviruses (Human cytomegalovirus and cytomegaloviruses of swine, monkeys and rodents); the gamma-herpesviruses (Epstein-Barr virus (EBV), Marek's disease virus, Herpes saimiri, Herpesvirus ateles, Herpesvirus sylvilagus, guinea pig herpes virus, Lucke tumor virus); the family Adenoviridae, including the genus Mastadenovirus (Human subgroups A,B,C,D,E and ungrouped; simian adenoviruses (at least 23 serotypes), infectious canine hepatitis, and adenoviruses of cattle, pigs, sheep, frogs and many other species, the genus Aviadenovirus (Avian adenoviruses); and non-cultivatable adenoviruses; the family Papoviridae, including the genus Papillomavirus (Human papilloma viruses, bovine papilloma viruses, Shope rabbit papilloma virus, and various pathogenic papilloma viruses of other species), the genus Polyomavirus (polyomavirus, Simian vacuolating agent (SV-40), Rabbit vacuolating agent (RKV), K virus, BK virus, JC virus, and other primate polyoma viruses such as Lymphotrophic papilloma virus); the family Parvoviridae including the genus Adeno-associated viruses, the genus Parvovirus (Feline panleukopenia virus, bovine parvovirus, canine parvovirus, Aleutian mink disease virus, etc). Finally, DNA viruses may include viruses which do not fit into the above families such as Kuru and Creutzfeldt-Jacob disease viruses and chronic infectious neuropathic agents (CHINA virus).

Infectious viruses of plants include insect or nematode transmitted viruses and those mechanically transmitted through handling, cutting, grafting, etc. Such viruses include, but are not limited to: tobacco rattle virus, pea early-browning virus, tobacco mosaic virus, cucumber green mottle mosaic virus, odontoglossum ringspot virus, ribgrass mosaic virus, Sammon's Opuntia virus, sann hemp mosaic virus, tomato mosaic virus, potato virus X cactus virus X, clover yellow mosaic virus, hydrangea ringspot virus, white clover mosaic virus, carnation latent virus, cactus virus 2, chrysanthemum virus B, passiflora latent virus, pea streak virus, potato virus M, potato virus S, red clover vein mosaic virus, potato virus Y, bean common mosaic virus, bean yellow mosaic virus, beet mosaic virus, clover yellow vein virus, cowpea aphid-borne mosaic virus, Columbian datura virus, henbane mosaic-virus, pea mosaic virus, potato virus A, soybean mosaic virus, sugar beet yellows viruses, sugar cane mosaic-virus, tobacco etch virus, watermelon mosaic virus (South African), alfalfa mosaic virus, pea enation mosaic virus, cucumber mosaic virus (S isolate), tomato aspermy virus, yellow cucumber mosaic virus, turnip yellow mosaic virus, cacao yellow mosaic virus, wild cucumber mosaic virus, Andean potato latent virus, belladonna mottle virus, dulcamara mottle virus, eggplant mosaic virus, ononis yellow mosaic virus, cowpea mosaic virus (SB isolate), bean pod mottle virus, broad bean stain virus, radish mosaic virus, red clover mottle virus, squash mosaic virus, true broad bean mosaic virus, tobacco ringspot virus, arabis mosaic virus, grapevine fanleaf virus, raspberry ringspot virus, strawberry latent ringspot virus, tomato black ring virus, tomato ringspot virus, etc. The type member of Group 12 is tobacco necrosis virus (A strain), tobacco necrosis virus Strain D, brome mosaic virus, broad bean mottle virus, cowpea chlorotic mottle virus, tomato bushy stunt virus, artichoke mottle crinkle virus, carnation Italian ringspot virus, pelargonium leaf curl virus, petunia asteroid mosaic virus, tomato spotted wilt virus, cauliflower mosaic virus (cabbage B isolate), dahlia mosaic virus. In addition to the above viruses the methods of this invention can be used to treat or inhibit plant viroids such as chrysanthemum chlorotic mottle viroid, potato spindle tuber viroid, chrysanthemum stunt viroid, citrus exocortis viroid, etc.

Fungi are eukaryotic organisms, only a few of which cause infection in vertebrate mammals. Because fungi are eukaryotic organisms, they differ significantly from prokaryotic bacteria in size, structural organization, life cycle and mechanism of multiplication. Fungi are classified generally based on morphological features, modes of reproduction and culture characteristics. Although fungi can cause different types of disease in subjects, such as respiratory allergies following inhalation of fungal antigens, fungal intoxication due to ingestion of toxic substances, such as amatatoxin and phallotoxin produced by poisonous mushrooms and aflotoxins, produced by aspergillus species, not all fungi cause infectious disease.

Infectious fungi can cause systemic or superficial infections. Primary systemic infection can occur in normal healthy subjects and opportunistic infections, are most frequently found in immuno-compromised subjects. The most common fungal agents causing primary systemic infection include blastomyces, coccidioides, and histoplasma. Common fungi causing opportunistic infection in immuno-compromised or immunosuppressed subjects include, but are not limited to, Candida albicans (an organism which is normally part of the respiratory tract flora), Cryptococcus neoformans (sometimes in normal flora of respiratory tract), and various Aspergillus species. Systemic fungal infections are invasive infections of the internal organs. The organism usually enters the body through the lungs, gastrointestinal tract, or intravenous lines. These types of infections can be caused by primary pathogenic fungi or opportunistic fungi.

Superficial fungal infections involve growth of fungi on an external surface without invasion of internal tissues. Typical superficial fungal infections include cutaneous fungal infections involving skin, hair, or nails. An example of a cutaneous infection is Tinea infections, such as ringworm, caused by Dermatophytes, such as microsporum or traicophyton species, i.e., Microsporum canis, Microsporum gypsum, Tricofitin rubrum. Examples of fungi include: Cryptococcus neoformans, Histoplasma capsulatum, Coccidioidies immitis, Blastomyces dermatitidis, Chlamydia trachomatis, Candida albicans.

Parasitic infections targeted by the methods of the invention include those caused by the following parasites Plasmodium falciparum, Plasmodium ovale, Plasmodium malariae, Plasmdodium vivax, Plasmodium knowlesi, Babesia microti, Babesia divergens, Trypanosoma cruzi, Toxoplasma gondii, Trichinella spiralis, Leishmania major, Leishmania donovani, Leishmania braziliensis and Leishmania tropica, Trypanosoma gambiense, Trypanosmoma rhodesiense and Schistosoma mansoni.

Other medically relevant microorganisms have been described extensively in the literature, e.g., see C. G. A Thomas, Medical Microbiology, Bailliere Tindall, Great Britain 1983, the entire contents of which is hereby incorporated by reference. Each of the foregoing lists is illustrative, and is not intended to be limiting.

The invention includes, in some aspects, methods of preventing and/or treating microbial infection in a subject. Such methods include administering a pharmaceutical agent or compound of the invention in an amount effective to prevent or treat a microbial infection in a subject. For example, a pharmaceutical compound that includes an MMPAP-12 molecule, as described herein, can be administered to prevent or treat a microbial infection in a subject. The effectiveness of treatment or prevention methods of the invention can be determined using standard diagnostic methods described herein.

The term “effective amount” of a MMPAP-12 polypeptide or nucleic acid refers to the amount necessary or sufficient to realize a desired biologic effect. For example, an effective amount of a MMPAP-12 polypeptide or nucleic acid for treating or preventing infectious disease is that amount necessary to prevent the infection with the microorganism if the subject is not yet infected or is that amount necessary to prevent an increase in infected cells or microorganisms present in the subject or that amount necessary to decrease the amount of the infection that would otherwise occur in the absence of the MMPAP-12 polypeptide or nucleic acid. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is effective to treat the particular subject. The effective amount for any particular application can vary depending on such factors as the disease or condition being treated, size of the subject, or the severity of the disease or condition. One of ordinary skill in the art can empirically determine the effective amount of a particular MMPAP-12 polypeptide or nucleic acid and/or other therapeutic agent without necessitating undue experimentation.

In some embodiments of the invention, the MMPAP-12 polypeptide or nucleic acid is administered in an amount effective to treat or prevent infectious disease. An effective amount is that amount which produces a physiological response that is greater than the response without the administration of the MMPAP-12 molecule. For example, in some embodiments of the invention, the physiological effect is a reduction in the number of cells infected with bacteria. An effective amount is that amount which produces a reduction in infected cells that is greater than the number of the infected cells without administration of the MMPAP-12 molecule. In other embodiments, the physiological result is a reduction in the number of bacteria in the body. The effective amount in this case is that amount which produces the reduction that is greater than the amount of reduction produced without administration of the MMPAP-12 molecule. In other embodiments the physiological result is a decrease in physiological parameters associated with the infection, e.g., lesions or other symptoms. For instance, a diagnosis of urinary tract infection is based on the presence and quantification of bacteria in the urine when greater than 10⁵ colonies per milliliter of microorganisms are detected in a mid-stream, clean-voided urine specimen. A reduction in this number to 10³ and preferably to fewer than 10² bacterial colonies per milliliter indicates that the infection has been eradicated.

The pharmaceutical compound or agent dosage may be adjusted by a physician or veterinarian, particularly in the event of any complication. A therapeutically effective amount typically varies from 0.01 mg/kg to about 1000 mg/kg, preferably from about 0.1 mg/kg to about 200 mg/kg, and most preferably from about 0.2 mg/kg to about 20 mg/kg, in one or more dose administrations for one or more days.

The absolute amount of a pharmaceutical compound that is administered will depend upon a variety of factors, including the material selected for administration, whether the administration is in single or multiple doses, and individual patient parameters including age, physical condition, size, weight, and the stage of the disease. These factors are well known to those of ordinary skill in the art and can be addressed with no more than routine experimentation.

The determination of whether treatment in a subject is effective, and/or whether the amount administered is a therapeutically effective amount can be done using routine methods known those of ordinary skill in the art. For example, diagnostic tests known to those of ordinary skill in the art or as described herein, may be used to assess the microbial infection status of a subject and evaluate the effectiveness of a pharmaceutical compound or agent that has been administered to the subject. A first determination of microbial infection may be obtained using one of the methods described herein (or other methods known in the art), and a subsequent determination of the presence of microbial infection in a subject may be done. A comparison of the presence of microbial infection, for example by determining the infection level/presence before and after administration of a pharmaceutical agent comprising an MMPAP-12 polypeptide or nucleic acid molecule of the invention, may be used to assess the effectiveness of administration of a pharmaceutical compound or agent of the invention as a prophylactic or a treatment of the microbial infection. The presence of indications of microbial infection in a subject that is above the indications in uninfected subjects may be an indication of a need for treatment intervention by administering a pharmaceutical agent described herein to prevent or treat a microbial infection.

The pharmaceutical agents of the invention may be administered alone, in combination with each other, and/or in combination with other anti-microbial drug therapies and/or treatments. These therapies and/or treatments may include, but are not limited to: surgical intervention, chemotherapy, and adjuvant systemic therapies. The type of anti-microbial drugs that may be administered in conjunction with the MMPAP-12 molecules of the invention will depend upon the type of microorganism with which the subject is infected or at risk of becoming infected. Examples of drugs that that may be administered in conjunction with the MMPAP-12 molecules of the invention include: antibacterial agents, antiviral agents, antifungal agents, and antiprotozoan agents, vaccines, etc. This list of agents is not meant to be limiting, and it will be understood by one of ordinary skill that additional antimicrobial agents can also be administered. When the other therapeutic agents are administered in conjunction with the MMPAP-12 molecules of the invention, they can be administered in the same or separate formulations, but are administered at the same time. The other therapeutic agents may also be administered sequentially with the MMPAP-12 polypeptide or nucleic acid, which means that the administration of the other therapeutic agents and the MMPAP-12 polypeptides and/or nucleic acids are temporally separated. The separation in time between the administration of these compounds may be a matter of minutes or it may be longer.

In some instances, a sub-therapeutic dosage of a second antibacterial agent may be administered in conjunction with an MMPAP-12 molecule of the invention. A “sub-therapeutic dose” as used herein refers to a dosage that is less than that dosage which would produce a therapeutic result in the subject. Thus, the sub-therapeutic dose of an anti-microbial agent is one that would not produce the desired therapeutic result in the subject in the absence of the MMPAP-12 molecule of the invention. Therapeutic doses of anti-bacterial agents are well known in the field of medicine for the treatment of infectious disease. These dosages have been extensively described in references such as Remington's Pharmaceutical Sciences, 18th ed., 1990; as well as many other medical references relied upon by the medical profession as guidance for the treatment of infectious disease.

In other embodiments of the invention, an MMPAP-12 molecule of the invention is administered on a routine schedule, but alternatively, may be administered as symptoms arise. A “routine schedule” as used herein, refers to a predetermined designated period of time. The routine schedule may encompass periods of time which are identical or which differ in length, as long as the schedule is predetermined. For instance, the routine schedule may involve administration of the MMPAP-12 molecule on a daily basis, every two days, every three days, every four days, every five days, every six days, a weekly basis, a monthly basis or any set number of days or weeks there-between, every two months, three months, four months, five months, six months, seven months, eight months, nine months, ten months, eleven months, twelve months, etc. Alternatively, the predetermined routine schedule may involve administration of the MMPAP-12 molecule on a daily basis for the first week, followed by a monthly basis for several months, and then every three months after that. Any particular combination would be covered by the routine schedule as long as it is determined ahead of time that the appropriate schedule involves administration on a certain day.

An MMPAP-12 polypeptide may be in the form of a polypeptide when administered to the subject or it may be encoded by a nucleic acid vector. If the nucleic acid vector is administered to the subject the protein is expressed in vivo. Minor modifications of the primary amino acid sequences of the MMPAP-12 polypeptides may also result in a polypeptide which has substantially equivalent functional activity, as compared to the unmodified counterpart polypeptide. Such modifications may be deliberate, as by site-directed mutagenesis, or may be spontaneous. Thus, nucleic acids having such modifications are also encompassed.

For administration of a MMPAP-12 nucleic acid in a vector, the nucleic acid encoding the MMPAP-12 polypeptide is operatively linked to a gene expression sequence, which directs the expression of the protein within a eukaryotic cell. The “gene expression sequence” is any regulatory nucleotide sequence, such as a promoter sequence or promoter-enhancer combination, which facilitates the efficient transcription and translation of the protein to which it is operatively linked. The gene expression sequence may, for example, be a mammalian or viral promoter, such as a constitutive or inducible promoter: Constitutive mammalian promoters include, but are not limited to, the promoters for the following genes: hypoxanthine phosphoribosyl transferase (HPTR), adenosine deaminase, pyruvate kinase, β-actin promoter and other constitutive promoters. Exemplary viral promoters that function constitutively in eukaryotic cells include, for example, promoters from the cytomegalovirus (CMV), simian virus (e.g., SV40), papilloma virus, adenovirus, human immunodeficiency virus (HIV), Rous sarcoma virus, cytomegalovirus, the long terminal repeats (LTR) of Moloney leukemia virus and other retroviruses, and the thymidine kinase promoter of herpes simplex virus. Other constitutive promoters are known to those of ordinary skill in the art. The promoters useful as gene expression sequences of the invention also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

In general, the gene expression sequence shall include, as necessary, 5′ non-transcribing and 5′ non-translating sequences involved with the initiation of transcription and translation, respectively, such as a TATA box, capping sequence, CAAT sequence, and the like. Especially, such 5′ non-transcribing sequences will include a promoter region which includes a promoter sequence for transcriptional control of the operably joined MMPAP-12 nucleic acid. The gene expression sequences optionally include enhancer sequences or upstream activator sequences as desired.

As used herein, the nucleic acid sequence encoding the protein and the gene expression sequence are said to be “operably linked” when they are covalently linked in such a way as to place the expression or transcription and/or translation of the antigen coding sequence under the influence or control of the gene expression sequence. Two DNA sequences are said to be operably linked if induction of a promoter in the 5′ gene expression sequence results in the transcription of the gene sequence and if the nature of the linkage between the two DNA sequences does not (1) result in the introduction of a frame-shift mutation, (2) interfere with the ability of the promoter region to direct the transcription of the antigen sequence, or (3) interfere with the ability of the corresponding RNA transcript to be translated into a protein. Thus, a gene expression sequence would be operably linked to a specific nucleic acid sequence if the gene expression sequence were capable of effecting transcription of that nucleic acid sequence such that the resulting transcript is translated into the desired protein or polypeptide.

As described herein, the compositions of the invention may be delivered to the subject or other target cells and tissues alone or in association with one of a variety of available vectors. In its broadest sense, a “vector” is any vehicle capable of facilitating the transfer of the compositions to the target cells. The vector generally transports the nucleic acid to the target cells with reduced degradation relative to the extent of degradation that would result in the absence of the vector. In general, the vectors useful in the invention are divided into two classes: biological vectors and chemical/physical vectors. Biological vectors and chemical/physical vectors are useful for delivery/uptake of nucleic acids by a target cell.

Biological vectors include, but are not limited to, plasmids, phagemids, viruses, other vehicles derived from viral or bacterial sources that have been manipulated by the insertion or incorporation of nucleic acid sequences, and free nucleic acid fragments which can be attached to nucleic acid sequences. Viral vectors are a preferred type of biological vector and include, but are not limited to, nucleic acid sequences from the following viruses: retroviruses, such as: Moloney murine leukemia virus; Harvey murine sarcoma virus; murine mammary tumor virus; Rous sarcoma virus; adenovirus; adeno-associated virus; SV40-type viruses; polyoma viruses; Epstein-Barr viruses; papilloma viruses; herpes viruses; vaccinia viruses; polio viruses; and RNA viruses such as any retrovirus. One can readily employ other viral vectors not named but known in the art.

Preferred viral vectors are based on non-cytopathic eukaryotic viruses in which non-essential genes have been replaced with a nucleic acid of interest. Non-cytopathic viruses include retroviruses, the life cycle of which involves reverse transcription of genomic viral RNA into DNA with subsequent proviral integration into host cellular DNA. Retroviruses have been approved for human gene therapy trials. In general, the retroviruses are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., “Gene Transfer and Expression, A Laboratory Manual,” W. H. Freeman Co., New York (1990) and Murry, E. J. Ed. “Methods in Molecular Biology,” vol. 7, Humana Press, Inc., Clifton, N.J. (1991).

Another preferred virus for certain applications is the adeno-associated virus, a double-stranded DNA virus. The adeno-associated virus can be engineered to be replication-deficient and is capable of infecting a wide range of cell types and species. It further has advantages, such as heat and lipid solvent stability; high transduction frequencies in cells of diverse lineages; and lack of superinfection inhibition thus allowing multiple series of transductions. Reportedly, the adeno-associated virus can integrate into human insertional mutagenesis and variability of inserted gene expression. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other biological vectors include plasmid vectors. Plasmid vectors have been extensively described in the art and are well known to those of skill in the art. See e.g., Sambrook et al., “Molecular Cloning: A Laboratory Manual,” Second Edition, Cold Spring Harbor Laboratory Press, 1989. In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA.

It has recently been discovered that gene-carrying plasmids can be delivered to the immune system using bacteria. Modified forms of bacteria that is resistant to antimicrobial effects of the MMPAP-12 molecule of the invention, such as. Salmonella, can be transfected with the plasmid and used as delivery vehicles. The bacterial delivery vehicles can be administered to a host subject orally or by other administration means. The bacteria deliver the plasmid to immune cells, e.g. B cells, dendritic cells, likely by passing through the gut barrier. High levels of immune protection have been established using this methodology. Such methods of delivery are useful for the aspects of the invention utilizing systemic delivery of the MMPAP-12 nucleic acid.

In addition to the biological vectors, chemical/physical vectors may be used to deliver an MMPAP-12 nucleic acid or polypeptide to a target cell and facilitate uptake thereby. As used herein, a “chemical/physical vector” refers to a natural or synthetic molecule, other than those derived from bacteriological or viral sources, capable of delivering the nucleic acid to a cell.

A preferred chemical/physical vector of the invention is a colloidal dispersion system. Colloidal dispersion systems include lipid-based systems including oil-in-water emulsions, micelles, mixed micelles, and liposomes. A preferred colloidal system of the invention is a liposome. Liposomes are artificial membrane vessels, which are useful as a delivery vector in vivo or in vitro. It has been shown that large unilamellar vessels (LUV), which range in size from 0.2-4.0 μm can encapsulate large macromolecules. RNA, DNA, and intact virions can be encapsulated within the aqueous interior and be delivered to cells in a biologically active form (Fraley, et al., Trends Biochem. Sci., (1981) 6:77).

Liposomes may be targeted to a particular tissue by coupling the liposome to a specific ligand such as a monoclonal antibody, sugar, glycolipid, or protein. Ligands which may be useful for targeting a liposome to a specific type of cell include, but are not limited to: intact or fragments of molecules which interact with the cell type's cell-specific receptors and molecules, such as antibodies, which interact with the cell surface markers of cells. Such ligands may easily be identified by binding assays well known to those of skill in the art. Additionally, the vector may be coupled to a nuclear targeting peptide, which will direct the vector to the nucleus of the host cell.

Lipid formulations for transfection are commercially available from QIAGEN, for example, as EFFECTENE™ (a non-liposomal lipid with a special DNA condensing enhancer) and SUPERFECT™ (a novel acting dendrimeric technology).

Liposomes are commercially available from Gibco BRL, for example, as LIPOFECTIN™ and LIPOFECTACE™, which are formed of cationic lipids such as N-[1-(2, 3 dioleyloxy)-propyl]-N, N, N-trimethylammonium chloride (DOTMA) and dimethyl dioctadecylammonium bromide (DDAB). Methods for making liposomes are well known in the art and have been described in many publications. Liposomes also have been reviewed by Gregoriadis, G. in Trends in Biotechnology, (1985) 3:235-241.

In one embodiment, the vehicle is a biocompatible microparticle or implant that is suitable for implantation or administration to the mammalian recipient. Exemplary bioerodible implants that are useful in accordance with this method are described in PCT International application no. Publication No. WO95/24929, entitled “Polymeric Gene Delivery System”. Pub. WO95/24929 describes a biocompatible, preferably biodegradable polymeric matrix for containing an exogenous gene under the control of an appropriate promoter. The polymeric matrix can be used to achieve sustained release of the exogenous gene in the patient.

The polymeric matrix preferably is in the form of a microparticle such as a microsphere (wherein the nucleic acid is dispersed throughout a solid polymeric matrix) or a microcapsule (wherein the nucleic acid is stored in the core of a polymeric shell). Other forms of the polymeric matrix for containing the nucleic acid include films, coatings, gels, implants, and stents. The size and composition of the polymeric matrix device is selected to result in favorable release kinetics in the tissue into which the matrix is introduced. The size of the polymeric matrix further is selected according to the method of delivery that is to be used, typically injection into a tissue or administration of a suspension by aerosol into the nasal and/or pulmonary areas. Preferably when an aerosol route is used the polymeric matrix and the nucleic acid and/or polypeptide is encompassed in a surfactant vehicle. The polymeric matrix composition can be selected to have both favorable degradation rates and also to be formed of a material which is bioadhesive, to further increase the effectiveness of transfer when the matrix is administered to a nasal and/or pulmonary surface that has sustained an injury. The matrix composition also can be selected not to degrade, but rather, to release by diffusion over an extended period of time.

Such sustained-release systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation. Another suitable compound for sustained release delivery is GELFOAM, a commercially available product consisting of modified collagen fibers.

In another embodiment the chemical/physical vector is a biocompatible microsphere that is suitable for delivery, such as oral or mucosal delivery. Such microspheres are disclosed in Chickering et al., Biotech. And Bioeng., (1996) 52:96-101 and Mathiowitz et al., Nature, (1997) 386:.410-414 and PCT Patent Application WO97/03702.

Both non-biodegradable and biodegradable polymeric matrices can be used to deliver the nucleic acid and/or polypeptide to the subject. Biodegradable matrices are preferred. Such polymers may be natural or synthetic polymers. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multi-valent ions or other polymers.

Bioadhesive polymers of particular interest include bioerodible hydrogels described by H. S. Sawhney, C. P. Pathak and J. A. Hubell in Macromolecules, (1993) 26:581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Compaction agents also can be used alone, or in combination with, a biological or chemical/physical vector to deliver nucleic acids. A “compaction agent”, as used herein, refers to an agent, such as a histone, that neutralizes the negative charges on the nucleic acid and thereby permits compaction of the nucleic acid into a fine granule. Compaction of the nucleic acid facilitates the uptake of the nucleic acid by the target cell. The compaction agents can be used alone, i.e., to deliver a nucleic acid in a form that is more efficiently taken up by the cell or, more preferably, in combination with one or more of the above-described vectors.

Other exemplary compositions that can be used to facilitate uptake by a target cell of the nucleic acid and/or polypeptide include calcium phosphate and other chemical mediators of intracellular transport, microinjiection compositions, electroporation and homologous recombination compositions (e.g., for integrating a nucleic acid into a preselected location within the target cell chromosome).

The MMPAP-12 nucleic acid and/or polypeptide and/or other therapeutics may be administered alone (e.g. in saline or buffer) or using any delivery vectors known in the art. For instance the following delivery vehicles have been described: Cochleates (Gould-Fogerite et al., 1994, 1996); Emulsomes (Vancott et al., 1998, Lowell et al., 1997); ISCOMs (Mowat et al., 1993, Carlsson et al., 1991, Hu et., 1998, Morein et al., 1999); Liposomes (Childers et al., 1999, Michalek et al., 1989, 1992, de Haan 1995a, 1995b); Live bacterial vectors (e.g., Salmonella, Escherichia coli, Bacillus calmatte-guerin, Shigella, Lactobacillus) (Hone et al., 1996, Pouwels et al., 1998, Chatfield et al., 1993, Stover et al., 1991, Nugent et al., 1998); Live viral vectors (e.g., Vaccinia, adenovirus, Herpes Simplex) (Gallichan et al., 1993, 1995, Moss et al., 1996, Nugent et al., 1998, Flexner et al., 1988, Morrow et al., 1999); Microspheres (Gupta et al., 1998, Jones et al., 1996, Maloy et al., 1994, Moore et al., 1995, O'Hagan et al., 1994, Eldridge et al., 1989); Nucleic acid vaccines (Fynan et al., 1993, Kuklin et al., 1997, Sasaki et al., 1998, Okada et al., 1997, Ishii et al., 1997); Polymers (e.g. carboxymethylcellulose, chitosan) (Hamajima et al., 1998, Jabbal-Gill et al., 1998); Polymer rings (Wyatt et al., 1998); Proteosomes (Vancott et al., 1998, Lowell et al., 1988, 1996, 1997); Sodium Fluoride (Hashi et al., 1998); Transgenic plants (Tacket et al., 1998, Mason et al., 1998, Haq et al., 1995); Virosomes (Gluck et al., 1992, Mengiardi et al., 1995, Cryz et al., 1998); Virus-like particles (Jiang et al., 1999, Leibl et al., 1998).

In other aspects, the invention relates to kits that are useful in the treatment of infectious disease. One kit of the invention includes a container housing an MMPAP-12 molecule of the invention and instructions for timing of administration of the MMPAP-12 molecule. In some embodiments, the MMPAP-12 molecule is provided for systemic administration, and the instructions accordingly provide for this. In other embodiments, the MMPAP-12 molecule is provided for topical administration, and the instructions accordingly provide for this. In some embodiments, the container housing the MMPAP-12 molecule is a sustained release vehicle that is used herein in accordance with its prior art meaning of any device that slowly releases the MMPAP-12.

The kit may include the MMPAP-12 molecule in a single container or it may be multiple containers or chambers housing individual dosages of the MMPAP-12 molecule, such as a blister pack. The kit also has instructions for timing of administration of the anti-microbial agent. The instructions would direct the subject having an infectious disease or at risk of an infectious disease to take the MMPAP-12 molecule at the appropriate time. For instance, the appropriate time for delivery of the medicament may be as the symptoms occur. Alternatively, the appropriate time for administration of the medicament may be on a routine schedule such as monthly or yearly.

In other aspects of the invention, a composition is provided. The composition includes an MMPAP-12 molecule of the invention formulated in a pharmaceutically acceptable carrier and present in the composition in an effective amount for preventing or treating an infection, e.g. a bacterial infection. The effective amount for preventing or treating an infectious disease is that amount that prevents, inhibits completely or partially infection or prevents an increase in the infection.

The pharmaceutical compositions of the invention contain an effective amount of an MMPAP-12 molecule and/or other therapeutic agents optionally included in a pharmaceutically-acceptable carrier. The termn “pharmaceutically-acceptable carrier” means one or more compatible solid or liquid filler, dilutants or encapsulating substances that are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

For any compound described herein a therapeutically effective amount can be initially determined in vitro and/or from cell culture assays and based on known effective amounts described herein in the Examples section. For instance the effective amount of MMPAP-12 molecules useful for preventing or treating a bacterial infection can be assessed using the in vitro assays. This type of assay can be used to determine an effective amount of the particular oligonucleotide for the particular infection type, subject, and the dosage can be adjusted upwards or downwards to achieve the desired levels in the subject. Therapeutically effective amounts can also be determined from animal models. The applied dose of the MMPAP-12 molecule can be adjusted based on the relative bioavailability and potency of the administered compound. Adjusting the dose to achieve maximal efficacy based on the methods described above and other methods are well known in the art and it is well within the capabilities of one of ordinary skill in the art.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

The MMPAP-12 molecules of the invention can be administered by any ordinary route for administering medications. For use in therapy, an effective amount of an MMPAP-12 molecule can be administered to a subject by any mode that delivers the MMPAP-12 molecule to the desired surface, e.g., mucosal, systemic, or topical. “Administering” the pharmaceutical composition of the present invention may be accomplished by any means known to the skilled artisan. Preferred routes of administration include but are not limited to oral, parenteral, intramuscular, intranasal, intratracheal, inhalation, ocular, vaginal, and rectal. Preferably, the pharmaceutical compositions of the invention are inhaled, ingested or administered by systemic routes. Systemic routes include oral and parenteral. Inhaled medications are preferred in some embodiments because of the direct delivery to the lung, e.g. when bacterial, viral or fungal agents are inhaled. Several types of metered dose inhalers are regularly used for administration by inhalation. These types of devices include metered dose inhalers (MDI), breath-actuated MDI, dry powder inhaler (DPI), spacer/holding chambers in combination with MDI, and nebulizers.

For oral administration, the compounds (i.e., MMPAP-12 molecules) can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from an insufflator, pressurized packs, a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. Techniques for preparing aerosol delivery systems are well known to those of skill in the art. Generally, such systems should utilize components which will not significantly impair the biological properties of the therapeutic, such as the antibacterial capacity of the MMPAP-12 molecules (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing aerosols without resort to undue experimentation. Alternatively, the compounds of the invention can be delivered as a dry powder composition containing, for example, the pure compound together with a suitable powder base (e.g., lactose, starch).

For intra-nasal administration, the compounds of the invention can be administered via nose drops, a liquid spray, such as via a plastic bottle atomizer or metered-dose inhaler. Exemplary atomizers are known to those of ordinary skill in the art. Drops, such as eye drops or nose drops, can be formulated with an aqueous or non-aqueous base which optionally further includes one or more dispersing agents, solubilizing agents or suspending agents. Apparatus and methods for delivering liquid sprays and/or drops are well known to those of ordinary skill in the art.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Alternatively, the active compounds may be in powder form for constitution with a suitable vehicle, e.g., sterile pyrogen-free water, before use.

For topical administration, the compounds (i.e., MMPAP-12 molecules) can be formulated readily by combining the active compound(s) with pharmaceutically acceptable carriers well known in the art. When the compositions of the invention are to be delivered via topical administration, the compounds can be administered as a pure dry chemical (e.g., by inhalation of a fine powder via an insufflator) or as a pharmaceutical composition further including a pharmaceutically acceptable topical carrier. Thus, the pharmaceutical compositions of the invention include those suitable for administration by inhalation or insufflation or for nasal, intraocular or other topical (including buccal and sub-lingual) administration.

For topical administration to the eye, nasal membranes or to the skin, the compounds according to the invention may be formulated as ointments, creams or lotions, or as a transdermal patch or intraocular insert or iotophoresis. For example, ointments and creams can be formulated with an aqueous or oily base alone or together with suitable thickening and/or gelling agents. Lotions can be formulated with an aqueous or oily base, and, typically, further include one or more emulsifying agents, stabilizing agent, dispersing agents, suspending agents, thickening agents, or coloring agents. (see, e.g., U.S. Pat. No. 5,563,153, entitled “Sterile Topical Anesthetic Gel.”, issued to Mueller, D., et al., for a description of a pharmaceutically acceptable gel-based topical carrier.

In general, the compounds of the invention are present in a topical formulation in an amount ranging from about 0.01% to about 30.0% by weight, based upon the total weight of the composition. Preferably, the compounds of the invention are present in an amount ranging from about 0.5 to about 30% by weight and, most preferably, the compounds are present in an amount ranging from about 0.5 to about 10% by weight. In one embodiment, the compositions of the invention comprise a gel mixture to maximize contact with the surface of the skin or membrane and to minimize the volume and dosage necessary. GELFOAM ® (a methylcellulose-based gel manufactured by Upjohn Corporation) is a preferred pharmaceutically acceptable topical carrier. Other pharmaceutically acceptable carriers include iontophoresis for transdermal drug delivery.

In one aspect of the invention, the compounds of the invention are formulated in a composition for delivery in the oral cavity. An exemplary pharmaceutically acceptable topical carrier for the sustained release of an antimicrobial in the oral cavity is a polyvinyl alcohol matrix such as that described in U.S. Pat. No. 5,520,924, entitled “Methods and articles for administering drug to the oral cavity”, issued to Chapman, R., et al. Alternative formulations suitable for topical administration in the mouth or throat include lozenges comprising the compound(s) of the invention in a flavored base, usually sucrose and acacia or tragacanth; pastilles comprising the compound(s) in an inert base such as gelatin and glycerin or sucrose and acacia; and mouthwashes comprising the active ingredient in a suitable liquid carrier. Other suitable carriers for delivery to the oral cavity or other topical surface are known to one of ordinary skill in the art.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The MMPAP-12 molecules may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The invention also, in some aspects, to the use of the MMPAP-12 polypeptides of the invention in materials. The MMPAP-12 polypeptides can be mixed in with the material, for example during manufacturing of the material or at a subsequent time. In addition, a MMPAP-12 polypeptide can be applied to the surface of a material, either during manufacturing or at a subsequent time. As used herein, the term “suitable material” means material with which the polypeptides can be applied, thereby incorporating an antimicrobial activity in/on the material. For example, a gauze pad on a bandage can be manufactured with MMPAP-12 polypeptide in or on the gauze, and/or an MMPAP-12 ointment can be applied to the gauze thereby incorporating antimicrobial activity to the gauze. Examples of suitable materials in which MMPAP-12 polypeptides may be used, include, but are not limited to: foods, liquids, an instrument (e.g. surgical instruments), a bead, a film, a monofilament, an unwoven fabric, sponge, cloth, a knitted fabric, a short fiber, a tube, a hollow fiber, an artificial organ, a catheter, a suture, a membrane, a bandage, and gauze. The MMPAP-12 polypeptide may be applied or mixed into numerous other types of materials that are suitable for use in medical, health, food safety, or environmental cleaning activities.

The invention also relates in part to methods to prevent contamination of materials and methods to decomtaminate materials using the MMPAP-12 polypeptides of the invention.

In other aspects the invention involves preventing and/or treating microbial contamination of materials. A “material” as used herein is any liquid or solid material including, but not limited to: blood, tissue, bodily fluids, and tissue-processing equipment, including but not limited to: equipment for food processing, medical equipment, equipment for tissue transplant processing, and equipment for cell or bodily fluid processing. In some embodiments of the invention, the material is aqueous. In some embodiments, the material is water, an example of which, although not intended to be limiting, is drinking water. The invention also involves preventing and/or treating microbial contamination in blood, bodily fluids, cells, and tissue samples, including those from live human subjects and cadavers, as well as live animals and animal tissues and cells processed as food, cosmetics, or medication. As used herein, the term “contamination” means contact between the material and a living microorganism.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

All references, patents and patent publications that are recited in this application are incorporated in their entirety herein by reference.

### EXAMPLES

Introduction

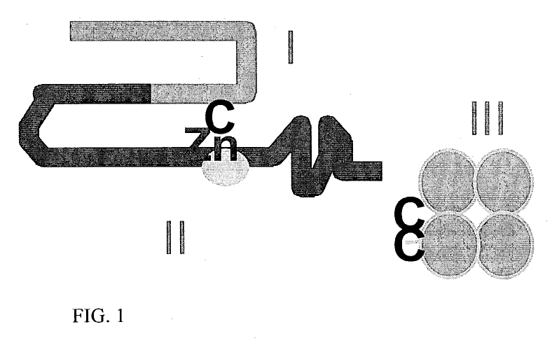

Macrophage elastase has potent proteinase activity against several constituents of the matrix including the highly insoluble elastin. Macrophage elastase has been cloned and confirmed by its predicted sequence to be a unique member of the matrix metalloproteinase (MMP) family and designated matrix metalloproteinase 12, (MMP-12) (FIG. 1). MMP-12 encodes a 54 kDa proenzyme consisting of three common domains: a pro-enzyme amino terminal domain, a zinc binding catalytic domain, and a hemopexin like carboxy terminal domain.

### Example 1

Antimicrobial Activity of MMP-12

We investigated the role MMP-12 plays in host defense against bacteria and identified a novel use of (MMP-12), as a macrophage antimicrobial agent. We have determined that MMP-12 has direct antimicrobial activity against gram-positive and gram-negative bacteria, and that MMP-12 has a novel intracellular and non-catalytic mechanism contained in its c-terminal hemopexin domain. To test for a function of MMP-12 in host defense, MMP-12−/− mice and wild-type littermates (MMP-12+/+) received infectious challenges to macrophage rich environments using a prototypical gram positive bacterium, S. aureus.

Methods

Mice: MMP-12 deficient mice, generated by gene targeting, and wild-type littermates, in a 129 Sv/Ev background, were used throughout all experiments. Mice were housed in pathogen free derived and barrier maintained facility. Adult mice ages >20 weeks were used for these experiments and matched for age and sex. Animal use was conducted in accordance with the institutional guidelines of Washington University.

Bacteria. Staphylococcus aureus used in these experiments was a clinical isolate. We chose to use this clinical isolate of S. aureus in our studies because a murine model of infection has been well studied. S. aureus was grown in tryptic soy broth (TSB, Difco, Detroit, Mich.) for 18 h at 37° C. A 1:10 dilution of S. aureus was placed in fresh TSB for mid-log-phase growth. S. aureus was then centrifuged at 2000×g for 10 minutes and washed in sterile phosphate buffered saline (PBS) twice and diluted in PBS. The concentration of bacteria in PBS was determined by measuring the amount of absorbance at 540 nm. A standard of absorbencies based on known colony-forming units (CFU) was used to calculate the inoculum concentration quantity was confirmed by 1/100 dilution and next day CFU.

Peritonitis model: Mice were subjected to an intraperitoneal injection of S. aureus. Mice were followed for a two-week period. Mice demonstrating signs of respiratory difficulty or distress were euthanized according to Washington University guidelines. LD50 was determined for both types of mice.

Hematogenous Infection: Wild-type and MMP-12−/− mice were anesethized using 2.5% avertin. S. aureus in 400 μl of PBS was injected via tail vein. The mice mortality curve was followed over a two week time period. Mice exhibiting signs of distress were euthanized and counted as a mortality. Mice received a hematogenous injection of S. aureus and euthanized at 2 and 24 hours. At the time of sacrifice, lungs were flushed with one ml of sterile normal saline (NS) and removed aseptically and placed in 1 ml of sterile saline. Left lung, kidney, and spleen were homogenized with a tissue homogenizer under a vented hood. Homogenates were placed on ice, and serial 1/10 and 1/100 dilutions were made. Ten microliters of each dilution were plated on LB agar plates (Difco) and incubated for 18 h at 37° C., and then the colonies forming units were counted.

Pneumonia model: MMP-12−/− mice were anesthetized with intraperitoneal injection of 0.1-0.2 ml of 2.5% avertin. Trachea was isolated by sterile technique. S. aureus, prepared as described above in 100 ml, was injected into the trachea using a 30-gauge needle. The injection site was left opened and mice were observed daily for signs of distress. Mice that showed signs of respiratory difficulty, and inactivity over a two-week time course were euthanized according to Washington University guidelines.

Lung Bacterial Burden: MMP-12−/− and wild type littermates received intratracheal injection of S. aureus as described above. Mice were euthanized at 2 and 24 hours after injection. The left lung was removed using sterile technique and homogenized as described above. The right lung was inflated to 25-cm and fixed with 10% buffered formalin. The left lung was homogenized in 1 ml sterile PBS for CFU count as described above.

Histology: Tissues were perfused, inflated (for lung only), fixed in 10% buffered formalin, and processed for paraffin sections. Routinely, 5-mm paraffin sections were cut and stained with hematoxylin and eosin and Brown and Brenn bacterial stain using standard methods.

Peritoneal Macrophages: Mice were injected with 1 ml of sterile Brewers thioglycol media. Peritoneal macrophages were obtained by peritoneal lavage with 10 cc of iced normal saline instilled into the peritoneal cavity with a 21-gauge needle and withdrawn. Lavage was repeated for a total volume of 20 ml of lavage fluid. Peritoneal lavage fluid was centrifuged at 4° C. for 10 min at 600×g. Cells were resuspended in condition media (Dulbeco's Modified Eagles Media, 10% fetal bovine serum, Streptomycin 50 μg/ml, penicillin 50 μmg/ml). Cytospin slides of this suspension were then prepared and stained (Diff-Quik Stain set; Dade Behring, Newark, Del.), and differential cell counts were determined using a high-power microscope. The absolute number of a leukocyte subtype was determined by multiplication of the percentage of that cell type by the total number of cells. Cultures were >95% peritoneal macrophages. Cells were plated in sterile 24-well plates (Costar) at a concentration of 2.5×10⁵/well. The following day, cells were washed to remove dead and non-adherent cells and antibiotic-free media was added.

Macrophage Intracellular Killing Experiments: S. aureus was added to macrophage cultures at a concentration of 10 bacterium per macrophage and centrifuged at 400×g for 5 minutes. Co-cultures were incubated at 37° C. humidified in a 5% (vol/vol) CO₂ injected incubator for one hour, to allow for adequate phagocytosis. Co-cultures were washed with sterile PBS×3 and an antibiotic condition media (100 μg/ml gentamicin, 100 μg/ml penicillin, 100 μg/ml streptomycin) was added. Cultures were incubated for 30 minutes to kill extracellular and membrane bound bacteria. After 30 minutes, time course was started and at each time point condition media was removed, cells were washed and then permeabilized with 200 μl of sterile 0.2% Triton PBS solution then scraped. Cell lysates were diluted 1/10 and 1/100 in sterile PBS and plated on LB agar plates and incubated for 18 hours at 37° C. for CFU count.

Immunoelectron microscopy: Peritoneal macrophages were isolated using the previously described method. Macrophages (2×10⁶) were cultured in Teflon coated wells in DMEM, 10% fetal bovine serum antibiotic free media. Staph aurcus (6×10⁶ CFU) added to macrophages for two hour incubation. Co-culture was stopped and cells were fixed with iced 5% glutaraldehyde PBS solution.

Recombinant Protein: MMP-12 carboxyterminal protein was generated using PET expression system. The primers utilized were 5′ primer ttttatggatatcagtccaccatcaact (SEQ ID NO:34) and 3′ primer ttttagaattcgaacaaccaaaccagcttgt (SEQ ID NO:35). MME carboxy terminal was directionally cloned into PET 20 b plasmid with EcoR1 and EcoRV cloning sites. The carboxy terminal was tagged with 6×histidine, used for purification and detection. Plasmid was transfected into BL21(DE3)LysE and grown to an O.D. 0.6 (Invitrogen, Carlsbad, Calif.). Culture was stimulated with 1 mM WPTG and grown for 16 hours. Cells were spun at 5,000×g for 15 minutes. Pellet was resuspended in 6M urea and purified under denaturing conditions. Recombinant protein was purified using cobalt histidine binding resin (Chemicon, Temecula, Calif.). Protein was eluted under nondenaturing condition using 50 mM sodium phosphate 300 mM NaCl pH 2.0 elution buffer. Production of protein was verified by western blotting using monoclonal antibody to 6 histidine residue (Invitrogen). Concentration of recombinant protein was determined using Bradford colorimetric assay. Purity was determined by Coomassie stained 10% PAGE.

In Vitro Antimicrobial Activity: S. aureus in mid-log phase of growth was co-cultured with MMP-12 recombinant c-terminal protein in a 5% LB media. S. aureus co-culture was incubated for 60 minutes with doses of MMP-12 C-terminal. Aliquots of cultures were diluted in PBS at 1:10 and 1:100 dilution. Dilutions were plated on LB agar plates for 18 hour incubation at 37° C. Controls consisted of column fractions that lacked MMP-12 carboxy terminal determined by immunoblotting.

Results

MMP-12−/− Mice Have Increased Mortality during Bacterial Peritonitis To confirm a function of MMP-12 in host defense, MMP-12−/− mice and wild type littermates (MMP-12+/+) received infectious challenges to macrophage rich environments using a prototypical gram positive bacterium, S. aureus. MMP-12−/− and MMP-12+/+mice received an intraperitoneal inoculation of S. aureus (4×10⁸ CFU) and were followed for 72 hours. MMP-12−/− mice demonstrated clinical signs of sepsis consisting of decreased activity, ruffled fur, and labored respiration with a mortality rate of 100% compared to 72% for MMP-12+/+ mice after 72 hours. Mice were then challenged with a gram-negative bacteria, E. coli (K1) (1×10⁸ CFU), a more typical peritoneal pathogen. Similar to S. aureus, MMP-12−/− mice had increased susceptibility to E. coli peritonitis compared to MMP-12+/+ mice. MMP-12−/− and MMP-12+/+ mice had mortality rate after 72 hours of 60% versus 40% respectively. These results demonstrated a novel function for MMP-12 for the improvement of survival during gram-positive and gram-negative bacterial peritonitis.

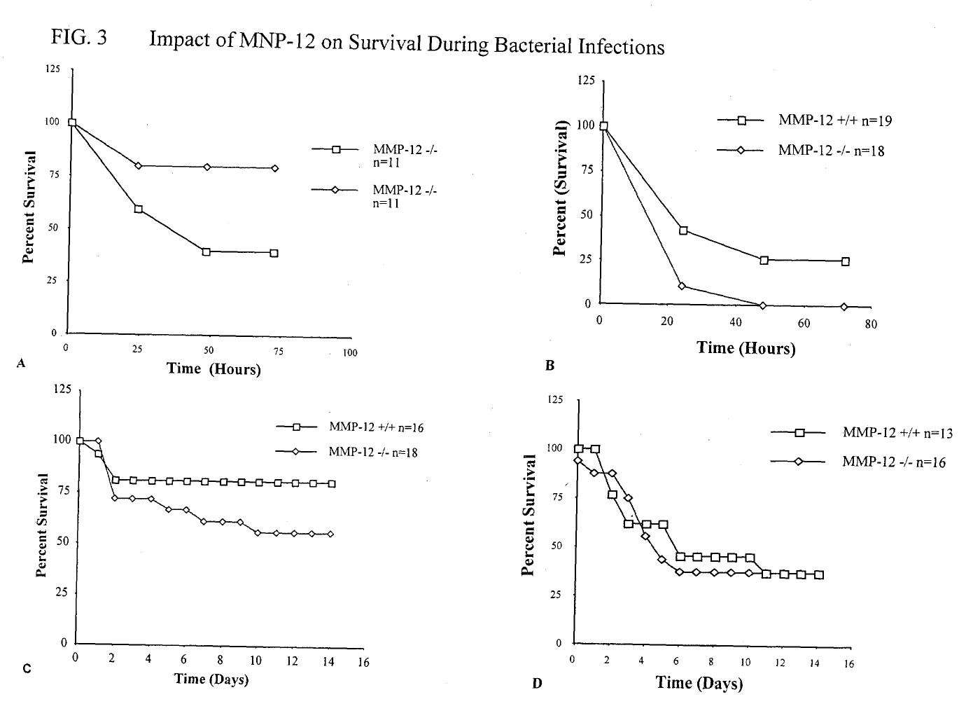

FIG. 3 demonstrates that MMP-12−/− mice have impaired survival during bacterial infections against gram positive and gram negative bacteria. FIG. 3A shows results obtained when MMP-12−/− and MMP-12+/+ mice (n=11) mice were injected into the peritoneum with E. coli (1×10⁸ CFU). Mice were observed for 72 hours for signs of distress and differences in mortality. FIG. 3B shows the results when a second group of mice (n=18 and n=19 respectively) were injected into the peritoneum with S. aureus (4×10⁸ CFU) and observed for 72 hours for distress and mortality. FIG. 3C shows the results of intratracheal injection of MMP-12−/− and MMP-12+/+ mice (n=16 and n=18 respectively) with S. aureus (4×10⁸ CFU). Mice followed for signs of infection and respiratory difficulty. FIG. 3 C shows the results of Tail vein inoculation of MMP-12+/+ and MMP-12−/− mice (n=13 and 16) with S. aureus (1×10⁸ CFU) observed for two weeks following previously described parameters.

MMP-12−/− Mice have Increased Mortality during S. aureus Pneumonia but not Hematogenous Infection.

Results from the peritonitis experiments demonstrated a role for MMP-12 in the setting of peritonitis. To confirm that other macrophage-containing organs, such as the lung, would demonstrate similar dependence on MMP-12 for survival during bacterial challenge, S. aureus (1×10⁸ CFU) was instilled into the pulmonary parenchyma via intratracheal injection. MMP-12−/− mice again showed signs of bacterial sepsis, as previously described, while MMP-12 +/+ mice demonstrated fewer and milder response to the challenge. Survival differences for the two strains of mice revealed a two-week mortality rate of 44% for MMP-12−/− mice with the majority of deaths occurring during the first 48 hours compared to a 19% mortality rate for MMP-12+/+ mice.

To define the impact of MMP-12 during a systemic infection, mice were inoculated hematogenously with S. aureus (4×10⁸ CFU). Survival rates for two weeks did not reveal differences between MMP-12−/− and MMP-12+/+ with both groups of mice having a mortality rate of 62%. Results from the hematogenous survival suggested that MMP-12, although improving survival during peritonitis and pneumonia, does not exert its host defense activity when bacteria circumvent macrophages.

MMP-12−/− Mice Have Impaired Pulmonary Clearance of Bacteria

To confirm that MMP-12 deficiency contributed to murine death during bacterial infection due to a macrophage impaired clearance of bacteria the following experiments were performed. The requirement of macrophages and MMP-12 in the clearance of bacteria in organs with varying quantities of tissue macrophages was tested. To examine whether MMP-12 had a regional clearance of bacteria based on the presence of tissue macrophages and not due to a systemic response such as the release of pro-inflammatory cytokines. MMP-12−/− and MMP-12+/+ mice were hematogenously infected (n=12 each group) with a sub-lethal dose of S. aureus (1×10⁶ CFU). Mice were euthanized at 2 and 24 hours for harvesting of spleen, kidney, and lung. Tissues were homogenized and diluted for CFU count. Results from this experiment demonstrated similar bacterial burden in spleen and kidney at both 2 and 24 hours for both groups of mice. Lung cultures revealed a larger bacterial load at 2 hours and by 24 hours MMP-12−/− mice had 5 fold more bacteria than MMP-12+/+ mice. MMP-12 +/+ mice had lower levels at both 2 and 24 hours with a trend toward bacterial clearance. These experiments confirmed that although MMP-12 did not affect survival during hematogenous infection, it had a role in the clearance of infection from the lung, a macrophage rich organ.

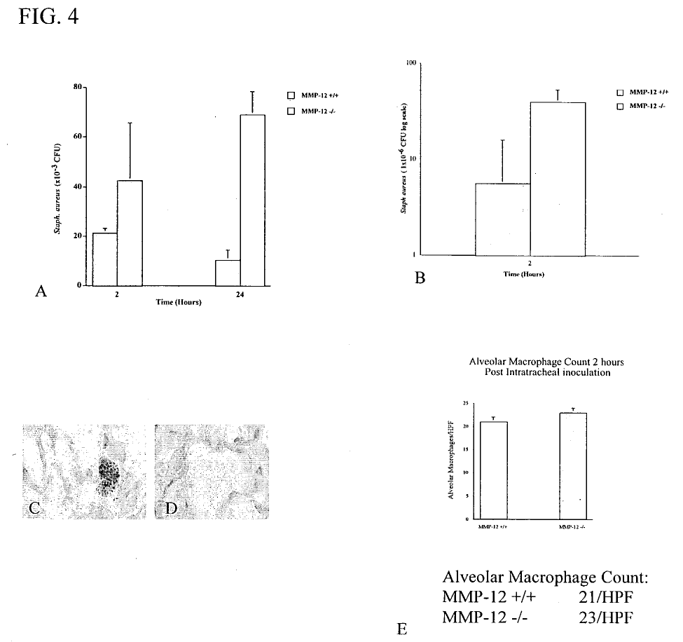

FIG. 4 illustrates impaired bacterial clearance from the lungs of MMP-12−/− mice compared to MMP-12+/+ mice. FIG. 4A shows the bacterial load in the lungs of MMP-12+/+ and MMP-12−/− mice after hematogenous inoculation of S. aureus (10⁶ CFU). FIG. 4B shows the bacterial load from the lungs of MMP-12+/+ and MMP-12−/− mice after sub-lethal intratracheal inoculation of S. aureus (CFU) at 2 and 24 hours. FIG. 4C shows a high power microscopy (×1000) image of lung tissue from MMP-12−/− and MMP-12+/+ mice two hours after bacterial challenge. Lung tissue stained with Brown and Brenn bacterial stain (gram positive bacteria stain dark).

To further determine pulmonary dependence on macrophage and MMP-12 to clear bacteria, MMP-12−/− and MMP-12+/+ mice (n=12 each group) were challenged with an intratracheal sub-lethal dose of S. aureus (6×10⁷ CFU). Lungs were harvested at 2 and 24 hours, similar to the hematogenous challenge. The results of this experiment demonstrated a larger bacterial load in the lungs of MMP-12−/− mice at 2 hours with a 10-fold increase in bacteria compared to MMP-12+/+ mouse lungs. At the 24-hour time point both groups of mice were able to clear bacteria. Lung histology from the groups of mice did not show any significant difference in neutrophil numbers or macrophages at either 2 or 24 hours after the inoculation. Lung tissue stained for bacteria demonstrated bacteria were concentrated inside alveolar macrophages in the MMP-12−/− mice at the two hour time point and not in the MMP-12 +/+ mice lungs consistent with our CFU counts. Previous reports have shown decreases in neutrophil recruitment in immunoglobulin mediated lung inflammation. Neutrophil and macrophage counts in the lungs of MMP-12−/− and MMP-12+/+ mice did not reveal any significant difference. These experiments demonstrated that MMP-12 had a role in bacterial clearance from a macrophage-containing organ.

MMP-12 is Important for Intracellular Macrophage Anti-microbial Activity

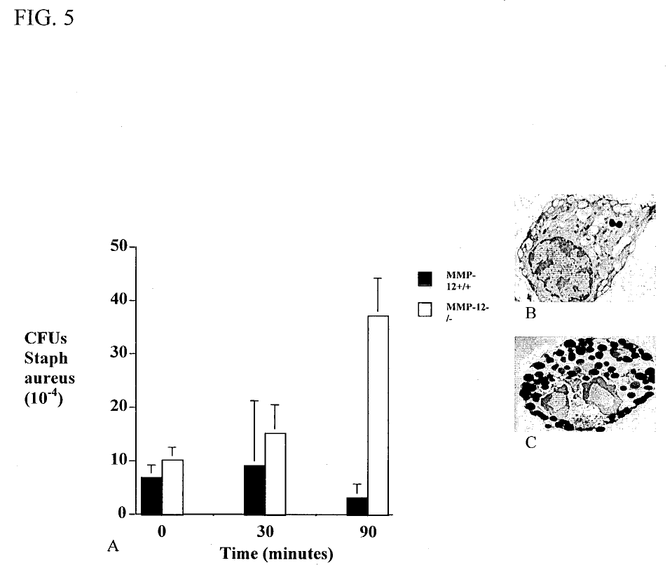

The intracelluar role of MMP-12 was examined in macrophage bacterial killing by co-culturing peritoneal macrophages from MMP-12−/− and MMP-12 +/+ mice with S. aureus using an antibiotic protection assay. Peritoneal macrophages were washed several times prior to the addition of bacteria to remove extracellular MMP-12. Bacteria were then co-incubated for one hour to allow for adequate phagocytosis. The co-culture was washed with PBS and an antibiotic media (gentamicin 100 μg/ml, penicillin 100 μg/ml) was added to kill extracellular and membrane bound bacteria. Over a 90-minute time course, macrophages were perrneabilized with Triton 0.2% and lysates were diluted and plated on LB agar plates for over night incubation and next day CFU count. Bacterial counts were then used as a representation of total viable intracellular bacteria. Results from these experiments revealed MMP-12−/− macrophages had 10 times more intracellular bacteria than wild type control (FIG. 5A) after a 90-minute time course. These findings have been repeated (n=6) with the consistent finding of impaired antimicrobial function of MMP-12−/− macrophages. Electron microscopy of the peritoneal macrophages co-incubated with S. aureus two hours revealed intracellular proliferation of bacteria in MMP-12−/− macrophages along with signs of cell death. MMP-12+/+ macrophages had significantly fewer bacteria (FIGS. 5B and 5C). Findings from both intracellular killing experiments and electron microscopy reveal a novel and previously unreported intracellular anti-microbial activity of MMP-12.

The results, which are illustrated in FIG. 5, indicate that MMP-12−/− macrophages have impaired intracellular killing. FIG. 5A shows results of an antibiotic protection assay of MMP-12+/+ and MMP-12−/− peritoneal macrophages co-cultured with S. aureus. Peritoneal macrophage co-cultures were incubated for one hour for phagocytosis after which extracellular and membrane bound bacteria were killed with antibiotic media (penicillin and gentamicin). Macrophages were lysed with Triton and intracellular quantity of bacteria was determined by CFU count of lysate. FIGS. 5B and 5C show results obtained when MMP-12+/+ and MMP-12−/− macrophages were co-incubated with S. aureus for two hours and then prepared for electron microscopy. High power electron microscopy of representative of MMP-12+/+ and MMP-12−/− macrophages show differences in the intracellular population of bacteria represented by dark spheres shown by the arrow.

MMP-12 Has Direct In Vitro Antimicrobial Activity

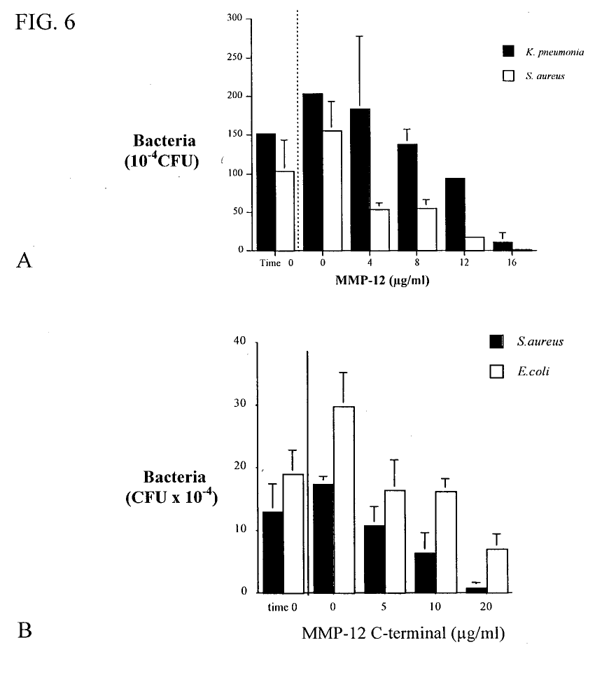

MMP-12's mechanism of action as a host defense protein was investigated. To test for direct activity, functional full-length recombinant human MMP-12 was incubated with S. aureus in a 5% LB culture. A dose response curve showed that MMP-12 had 90% bacterial kill at 16 μg/ml after 2-hour incubation (FIG. 6A). Similar antimicrobial activity and dose response were observed against K. pneumonia. MMPs 2,3,7,8, and 9 tested under similar conditions did not demonstrate this direct antimicrobial activity. MMP-12 enzymatic activity was not required for this antimicrobial effect. Full-length MMP-12 was inhibited under different conditions either with hydroxamic acid, an irreversible MMP inhibitor or heat denaturation and tested for antimicrobial activity. Neither the denatured MMP-12 or enzymatically inhibited enzyme lost its antimicrobial function. Furthermore, rMMP-12 active domain alone did not kill bacteria at similar doses and conditions. From these studies, we determined MMP-12 had a direct anti-microbial effect and its antimicrobial function was not dependent on its enzymatic activity and was located in a region outside the active domain.

MMP-12 C-terminal Has In Vitro Antimicrobial Activity

Because recombinant MMP-12 demonstrated a non-enzymatic in vitro antimicrobial activity, recombinant protein of the 26 kDa C-terminal domain was generated to isolate the region of antimicrobial activity. Recombinant C-terminal domain co-incubated with S. aureus showed similar activity and dose response as the full length MMP-12 with a 90% antimicrobial activity at 20 μg/ml (FIG. 6B). Recombinant c-terminal domains of MMP-2 and MMP-9 were also generated to test for the novelty of MMP-12 C-terminal antimicrobial function. When incubated under similar conditions only MMP-12 C-terminal domain demonstrated antimicrobial effects.

FIG. 6 shows results indicating that antimicrobial activity of MMP-12 is non-enzymatic and is located in the MMP-12 carboxy terminal domain. Recombinant full length human MMP-12 was co-incubated with S. aureus and K. pneumonia for 2 hours. Dose response curve was for recombinant murine carboxy terminal domain against S. aureus and E. coli after one-hour co-incubation.

MMP-12 Kills Bacteria by Disrupting Bacterial Membrane

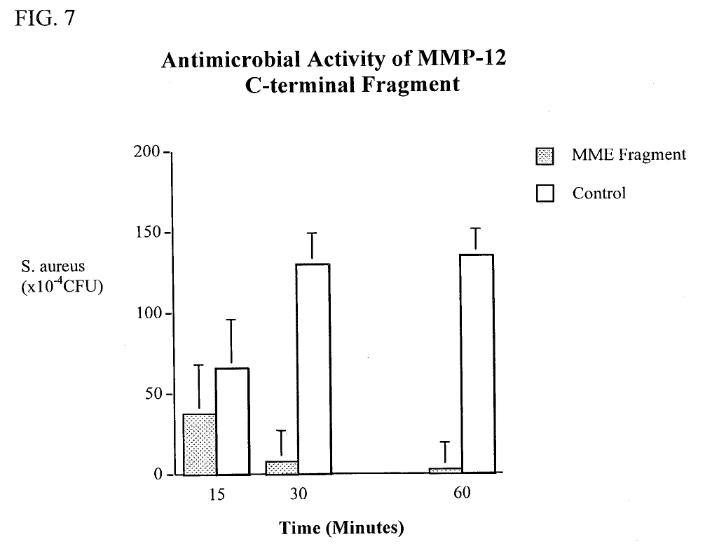

To confirm the ability of MMP-12 to disrupt the bacterial membrane, we co-incubated S. aureus with the MMP-12 C-terminal and added a hydrophilic fluorescent dye that is able to penetrate bacteria after disruption of the cell wall. Bacteria that developed cell leakage will fluoresce but intact bacteria will not. Results of these experiments revealed that bacteria that were incubated with MMP-12 C-terminal developed cell membrane leakage after one hour but bacteria incubated with control media did not show the same membrane leakage. The results, which are illustrated in FIG. 7, indicated that MMP-12 carboxy terminal has bactericidal activity by disrupting bacterial cell membrane against S. aureus. Bacteria incubated with MMP-12 C-terminal domain for one hour in the presence of membrane impermeant green fluorescent dye that increase in fluorescence by 100 fold when bound to DNA. Red fluorescent membrane permeant dye was also added for determination of total number bacteria present.

### Example 2

Roles of MMP-12 and Induction of MMP-12

Role of MMP-12 in Post Bone Marrow Transplant Lung Injury

Idiopathic pneumonia syndrome (IPS) is a significant non-infectious pulmonary injury syndrome, occurring after bone marrow transplantation, limiting the role of this life saving procedure. IPS, similar to pneumonia, is characterized by pulmonary infiltrates, fever and impaired oxygen exchange. Pulmonary biopsies from patients with IPS demonstrate alveolar damage with mononuclear infiltrates and alveolar hemorrhage. Immunohistochemistry from patients with the diagnosis of IPS revealed the presence of MMPs in the areas of alveolar damage and mononuclear infiltrates. MMP-12 and MMP-7 had the strongest expression.

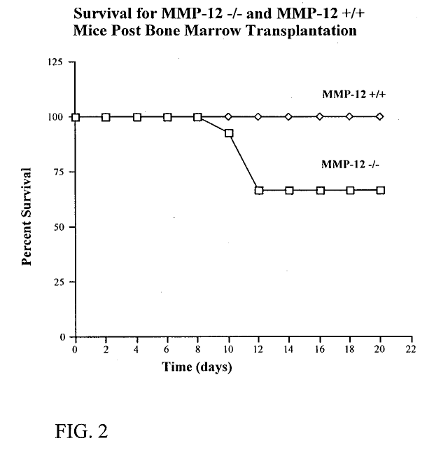

MMP-12 was found highly expressed in areas of monocytic infiltrates. To confirm the role of MMP-12 in this setting, a murine bone marrow transplant model system was developed using MMP-12−/− mice and wild type littermates. Mice were subjected to a lethal dose of external beam irradiation (10 cGY) and then received bone marrow from a donor mouse containing a single MHC mismatch. These studies revealed an increase in mortality for the MMP-12−/− mice of 40% starting at day , during the period of neutropenia (FIG. 2). In contrast, MMP-12+/+ littermates had a 100% survival during this same time period. Lung histology of MMP-12−/− mice contained areas of alveolar hemorrhage and mononuclear infiltrate compared mild inflammation and small vessel vasculitis in MMP-12+/+ mice. Bacterial stains of MMP-12−/− lung tissue showed gram-positive bacteria clustered in areas of inflammation and monocyte infiltrates. Tissue cultures identified the organism as Gemella morbillorum, a common bacterial colonizer of the oropharynx and gastrointestinal tract. Subsequent MMP-12−/− lung cultures grew gastrointestinal bacterial flora: E. faecalis, C. farmeri and E. cloacae. MMP-12−/− lung cultures had a 40% incidence of bacterial infection while wild-type lung cultures did not demonstrate the presence of bacteria by culture or histology. These studies identified a novel beneficial function for MMP-12 in the prevention of enteric bacterial dissemination during neutropenia after BMT.

Role of MMP-12 in Host Defense

To test for the role of MMP-12 in host defense, MMP-12−/− mice and wild-type littermates (MMP-12+/+) received infectious challenges to macrophage-rich environments using a prototypical gram-positive bacterium, S. aureus. MMP-12−/− and MMP-12+/+ mice received an intraperitoneal inoculation of S. aureus (4×10⁸ CFU). MMP-12−/− mice demonstrated clinical signs of sepsis consisting of decreased activity ruffled fur and labored respiration with a mortality rate of 100% after 72 hours compared to 72% for MMP-12+/+ mice. A similar difference in mortality between MMP-12−/− and MMP-12+/+ mice was observed after infection with E. coli (K1) (1×10⁸ CFU). These results demonstrated a novel role for MMP-12 in immunocompetent mice against both gram-positive and gram-negative bacterial infection during peritonitis.

Lung macrophages were next challenged via an intratracheal injection of S. aureus (3×10⁸ CFU). MMP-12−/− mice and MMP-12+/+, similar to the peritonitis model, demonstrated differences in susceptibility to the bacteria. MMP-12−/− mice developed signs of distress and had a mortality of 44% compared to 19% for MMP-12+/+ mice over two weeks. (FIG. 3). The majority of the deaths occurred with in the first 48 hours after inoculation.

In order to confirm a systemic role for MMP-12 in the clearance of bacteria, mice received a hematogenous injection of S. aureus (4 ×10⁸ CFU). In this infection model, MMP-12 did not impact overall survival between the groups of mice over a two-week time course. However, because MMP-12 is a macrophage specific proteinase and macrophages are tissue bound immune cells, an experiment was performed to confirm that MMP-12 dependent bacterial clearance would have regional distribution. Mice were inoculated with a sublethal dose of S. aureus (1×10⁶ CFU) and organs were removed to determine bacterial clearance during the early time period after infection. At 2 and 24 hours post inoculation, mice were euthanized and spleen, kidney, and lungs tissue cultures were obtained to determine bacterial burden in each organ. Results from this experiment demonstrated a similar bacterial burden in spleen and kidney from both MMP-12−/− and MMP-12+/+ mice. However, lung cultures revealed increasing quantity of bacterial load in the lungs of MMP-12−/− mice at 2 and 24 hours, while MMP-12+/+ mice had trend toward bacterial clearance (FIG. 4). MMP-12−/− mice also demonstrated an inability to clear bacteria from the lung after a sublethal challenge with S. aureus (6×10⁷ CFU). MMP-12+/+ and MMP-12−/− mice were challenged and lung cultures were obtained at 2 and 24 hours to determine bacterial burden. At 2 hours, MMP-12−/− lungs had 10 times more bacteria than MMP-12+/+ mice (FIG. 4), demonstrating MMP-12 is important for optimal macrophages clearance of bacteria during the initial stage of infection. Lung histology from MMP-12−/− mice demonstrated large pools of intracellular bacteria within alveolar macrophages, while MMP-12+/+ mice had few bacteria. These findings demonstrated a novel antimicrobial function for MMP-12, for macrophage antimicrobial activity. Histology from the pneumonia model suggested that MMP-12 has an intracellular function not previously reported. To confirm the intracellular function, peritoneal macrophages from MMP-12+/+ and MMP-12−/− mice were isolated and co-cultured with S. aureus using an antibiotic protection assay. Peritoneal macrophages were co-incubated with S. aureus in an antibiotic-free media for one hour to allow for adequate phagocytosis. Cells were washed with PBS and an antibiotic media (gentamicin 100 μg/ml, penicillin 100 μg/ml) was added to kill extra-cellular and membrane bound bacteria. Over a two-hour time course, macrophages were permeabilized with Triton 0.2% and lysates were diluted and plated on LB agar plates for over night incubation and next day CFU count. Bacterial counts were then used as a representation of total viable intracellular bacteria. Results from these experiments showed that MMP-12−/− macrophages had 10 times more intracellular bacteria than wild-type control (FIG. 4.) after a 90 minute time course. These findings were repeated (n=6) with the consistent finding of impaired antimicrobial function of MMP-12−/− macrophages. Electron microscopy of the peritoneal macrophages co-incubated with S. aureus two hours revealed intracellular proliferation of bacteria in MMP-12−/− macrophages along with signs of cell death. MMP-12 macrophages had significantly fewer bacteria (FIGS. 4C and D). These data demonstrated a novel intracellular anti-microbial activity of MMP-12 not described for any other MMP.

Recombinant full-length MMP-12 was generated and tested for direct antimicrobial activity against S. aureus. A dose-response curve showed that MMP-12 had 90% bacterial kill at 16 μg/ml after 2-hour incubation. Similar antimicrobial activity and dose response was observed against K. pneumonia. MMP 2, 3, 7, 8, and 9 tested under similar conditions did not demonstrate this direct antimicrobial activity. Results confirmed that MMP-12 enzymatic activity was not required for this antimicrobial effect. Pro-MMP-12 did not lose its anti-microbial activity in the presence of hydroxamic acid, a MMP inhibitor, or after heat inactivation. Furthermore, rMMP-12 active domain did not show anti-microbial activity. This suggested the anti-microbial activity is via a non-enzymatic linear peptide sequence, which is resistant to heat denaturation.

Experiments were focused on the MMP-12 C-terminal domain, which has only 40% homology to other MMPs and is autolytically cleaved. Recombinant murine MMP-12 C-terminal domain was generated and tested for direct antimicrobial activity against S. aureus. In vitro antimicrobial activity was observed with a 90% killing dose of 20 μg/ml. This data confirms a new function for MMP-12 as an antimicrobial peptide, and demonstrates the role of MMP-12 in the clearance of S. aureus from the lung. This novel function lies in the C-terminal domain and has a novel intracellular antimicrobial activity.

Bacterial Induction of MMP-12

Blood monocytes when differentiated into dendritic cells will increase mRNA levels after stimulation with lipopolysaccharide (LPS) and lipotechoic acids (LTA). Of the MMPs only MMP-12 and MMP-14 have been found to have significant increase in mRNA levels by genomic array screening. A similar experiment was performed using peritoneal macrophages and stimulated the macrophage culture with S. aureus cell wall component, lipoteichoic acid. The results consistently confirm that macrophages undergo histological changes after 48 hour co-incubation as well as increase extracellular expression of MMP-12.

### Example 3

Examination of MMP-12 Antimicrobial Activity

These studies confirm the bacterial range of activity and its mechanism of action of MMP-12, and confirm the peptide sequence responsible for the antimicrobial effect by generating segments of recombinant MMP-12 C-terminal and testing function.

The Antimicrobial Peptide Region of MMP-12 C-terminal Domain

Recombinant Protein

Antimicrobial peptides contain short peptide segments required for antimicrobial activity. The peptide segments containing antimicrobial activity are confirmed by dividing the domain into overlapping segments each covering approximately one third of the total length. This approach narrows the active site to about 60 amino acids. The C-terminal cDNA fragments are PCR amplified with EcoR1 and EcoRV restriction sites for cloning into the PET-20b cloning plasmid (Novagen Inc., Madison, Wis.). The PET-20b cloning plasmid contains a C-terminal 6×histidine tag for detection and purification. MMP-12 C-terminal constructs are transfected and expressed in BL21(DE3)LysE bacteria (Novagen) and induced with 1 mM IPTG and incubated for 12 hours. Peptides are solubilized in 6 M urea and purified using Talon resin (Clontech, Palo Alto, Calif.) and eluted under non-denaturing conditions using Bugbuster reagents (Novagen). Using this technique we have generated MMP-12 active and C-terminal domains. Peptide verification is performed by western blot analysis using anti-His Ab (Invitrogen) and by peptide sequencing (Brigham and Women's Hospital Biopolymer Lab Core Facility). Purity of the protein is determined by Coomassie stained 10% PAGE and concentration by Bradford assay.

Peptides are tested for antimicrobial activity against S. aureus as described above herein. S. aureus is grown in trypticase soy broth at 37° C. until exponential-phase growth. Bacteria are centrifuged and resuspended (10⁷ CFU/ml) in 10 mM potassium phosphate buffer pH 7.2 with 5% Luria-Bertani (LB) medium. S. aureus (10⁶ CFU/ml) are incubated with recombinant peptides in the buffer media in 96-well plates. S. aureus are incubated for two hours with serial dilutions of recombinant c-terminal. Aliquots of the suspension are diluted in PBS and plated on LB agar plates for 18 hr incubation at 37° C. and next day CFU count. Control for these experiments consists of BL21(DE3)LysE that underwent transfonrmation with the PET20b plasmid without MMP-12 C-terminal insert and is purified using similar conditions as recombinant protein. With respect to this particular experimnent, antimicrobial activity is defined as >90% reduction of S. aureus CFU at doses <50 μg/ml. All experimental conditions are done in triplicate. Standard deviation is calculated and results are tested for statistical significance using two-tailed T-test. Results are considered statistically significant with p value <0.05.

Peptides that demonstrate antimicrobial activity are further tested to determine physiological kinetics by performing time course and dose response experiments. Optimal conditions for antimicrobial activity are also determined. The effects of changing NaCl or Ca2+ and Mg2+ concentrations are tested as well and antimicrobial activity under range of pH in experimental conditions found in macrophage phagosomes and lysosomes is tested.

To further narrow the peptide sequence responsible for activity, peptides of the active segment consisting of 20 amino acids are generated (Brigham and Women's Hospital Biopolymer Lab Core Facility). Controls consist of random amino acid sequences of the peptides. Peptides are tested for antimicrobial activity using methods described herein. From this data the predicted secondary structure is determined by using commercially available programs i.e. Garnier-Doolittle (Geneworks). Similar method has been described in the generation of cathelicidins.

Antimicrobial peptides generally are cationic peptides that have amphipathic and alpha helical structures. Secondary structure allows for the insertion into bacterial cell walls and the production of pores. In order to determine if MMP-12 C-terminal has similar properties, mutants of the C-terminal are generated using site specific mutations (Stratagene, La Jolla, Calif.) to disrupt regions of alpha helical structure with proline residues and change predicted areas of amphipathic regions by inserting charged amino acids. To confirm the secondary structure x-ray crystallography of MMP-12 C-terminal is performed.

Confirming the Antimicrobial Function of MMP-12 C-terminal as a Bactericidal Protein

The data confirms a bactericidal activity of the C-terminal. The ability of C-terminal to directly kill bacteria is determined by using DAPI (Blue fluorescent live-cell stain) and SYTOX® (Green fluorescent dead-cell stain)(Molecular Probes, Eugene Oreg.). Sytox green fluorescent stain is a membrane impermeable stain. When bacterial membrane is disrupted the nucleus stains green indicating bacterial death. S. aureus is grown to logarithmic growth as described herein. S. aureus is incubated with c-terminal in a 5% LB media. Cells are centrifuged and resuspended in SYTOX and DAPI stain for 15 minutes at 37° C. Dead vs. live cells are determined by fluorescence microscopy and bacterial count/high powered field. The ratio of dead versus live bacteria is used to determine quantity of bacterial death. Flow cytometry is used to quantitate larger numbers of bacteria. Similar experiments are performed to assess bactericidal activity against E. coli.

Determining the Binding of MP-12 C-terminal to Bacterial Cell Wall

These experiments will assess pore formation as a possible first step by determining the ability of MMP-12 to bind directly to bacteria. Recombinant MMP-12 C-terminal fusion protein with a 6×His C-terminal tag has been generated. FITC-labeled antibody to the His tag (Invitrogen) is commercially available. Bacteria in mid-log phase of growth are incubated with the rMMP-12 C-terminal for one hour. Bacteria are centrifuged at 5000×g for 10 minutes and washed and resuspended in PBS. Bacteria are adhered to a glass slide and fixed in 10% buffered formalin. Bacteria are permeabilized with methanol at 4° C. and labeled with FITC antibody at 1:500 dilution. Binding is visualized using fluorescence microscopy. Localization experiments are conducted using bacteria transfected with red fluorescent protein, which allows for real-time quantitation of bacterial viability and visualization using fluorescence microscopy or confocal microscopy.

Experiments to confirm the ability of MMP-12 C-terminus to generate pores in bacteria cell walls. This is assessed by detecting the leakage of fluorescence marker from bacteria. S. aureus is incubated with calcein acetoxymethyl ester (calcein AM) (Molecular Probes) a lipid soluble nonfluorescent derivative of calcein that can cross membranes. Once inside the cytoplasm of target cells, calcein AM is hydrolyzed by cytoplasmic esterases, generating fluorescent calcein. S. aureus labeled with calcein is incubated with C-terminal. Membrane leakage is determined by change in fluorescence as detected by fluorometry. Total cell fluorescence is determined by flow cytometry, using standard methods.

A second method is to generate bacterial membrane liposomes. S. aureus is sonicated for 30 seconds to disrupt the bacteria cell wall. Bacterial membranes are allowed to fold into liposomes during a loading of fluorescent dye. Liposomes are incubated with MMP-12 C-terminal. During the co-incubation the bacterial liposomes are assessed for loss of membrane integrity by the loss of fluorescence. This technique eliminates loss of bacterial membrane integrity due to bacterial death.

Confirmation of MMP-12 C-terminal Domain Cleavage for Antimicrobial Activation

The data demonstrates that the full-length rMMP-12 has antibacterial properties. We have also found that the activity rests in the C-terminal domain and not in the active domain. MMP-12 has the unique property of autolytically cleaving its C-terminal domain. Antimicrobial peptides are produced as zymogens and require activation. MMP-12 can self cleave its C-terminal. This has been observed in the generation of recombinant protein as well as in the tissue culture. The requirement of the active domain for the processing of the full-length protein was confirmed by generating mutants of MMP-12. The active domain containing the zinc-binding site, is targeted by replacing histidine residues with lysine. Generation and purification of recombinant mutant follows previously described procedures. Mutant MMP-12 is tested for enzymatic activity against S. aureus and for antimicrobial activity.

The ability of enzymatic active MMP-12 to degrade the full-length mutant MMP-12 and release antimicrobial peptides is tested. Degradative products are tested for anti-microbial activity. Enzymatic active MMP-12 domain is incubated with mutant MMP-12 for 24 hours at 37° C. in Tris CaCl, and Zinc substrate buffer. Protein degradation is determined by Coomassie-stained 10% PAGE and with western blot analysis using anti-His Ab of pre- and post-digested protein. Peptide degradative products are purified using Talon resin. Peptide fragments are then tested for antimicrobial activity against S. aureus. Fragment separation is performed using sepharose gel size purification. Peptides that show activity are sequenced to determine location of cleavage (Brigham and Women's Hospital Biopolymer Lab Core Facility). Peptide fragments are separated by column chromatography.

Confirmation of the Spectrum of Cacteria Susceptible to MMP-12 Mediated Killing

The data demonstrates MMP-12 antimicrobial activity against S. aureus, E. coli (K1) and K. pneumonia(KPA). These bacteria are used as a positive control in the determination of recombinant MMP-12 peptides. Bacterial strains consist of bacteria found in the tissue cultures from the bone marrow transplant model as described herein. Pulmonary pathogens such as Streptococcus pneumoniae and Pseudomonas aeurogenosa are also tested. The following bacteria S. pneumonia, serotype 59 (ATCC #49619), H. influenzae ATCC (#35056), Enterococcus faecalis (ATCC #6057) and a clinical isolate of Pseuclomonas aeurogenosa are tested. Bacteria, twice passaged in vivo are grown in the appropriate culture media at 37° C. for logarithmic growth and washed twice in sterile phosphate potassium pH 7.2. Bacteria quantity is determined by optical density at 540 and as well as serial dilution with plating of LB agar media for overnight incubation and CFU count. Bacteria (1×10⁵ CFU) are be incubated in a 5% LB media with serial dilutions of recombinant MMP-12 C-terminal for two hours. Aliquot of cultures are diluted and plated on LB agar plate and incubated 37° C. for 18 hours for CFU count. Bacterial strains that demonstrate susceptibility are stained with Sytox dead cell bacterial stain to determine bacterial death.

The data demonstrates that MMP-12 has both gram-positive and gram-negative antimicrobial activity. Our experience in generating the MMP-12 proteins has given us insight into optimal conditions for the generation of recombinant MMP-12. MMP-12 proteins also are generated using baculovirus expression system, which has been successful for producing β-defensins.

### Example 4

Examination of MMP-12 Intracellular Antimicrobial Activity

Pulmonary macrophages are the most prevalent immune cell of the lung and serve as a significant innate immune cellular response to invading pathogens. Macrophages clear microbes through phagocytosis and intracellular degradation, which consists of oxygen dependent and independent pathways. Although not wishing to be bound to any particular theory or mechanism, our data indicates MMP-12 serves as an oxygen-independent constitutive host defense mechanism. Further examination of mechanism is assessed with cellular experiments that determine the intracellular trafficking of MMP-12 during rest and bacterial infection. The results of these studies confirm the intracellular role of MMP-12 during bacterial infection. The cellular microbiology of macrophages with phagocytized bacteria is examined. After macrophage engulfment of invading bacteria there are intracellular degradation mechanisms the macrophage use to kill bacteria. Bacteria have developed means to evade being killed such as the release of toxins that can induce apoptosis. Shigella and Salmonella are two examples of bacteria that secrete apoptosis inducing toxins, which activate caspases cascade. S. aureus also is able to induce apoptosis in endothelial cells and osteoblasts through the release of alpha-toxin. Electron microscopy of MMP-12−/− macrophages have shown signs of programmed cell death: nuclear condensation and excessive vacuolization and membrane disruption after the ingestion of S. aureus.

In co-culture experiments that MMP-12−/− macrophages have a greater loss of adherent macrophages compared to MMP-12+/+ macrophages. Electron microscopy of co-cultures showed the characteristic findings of apoptosis after two-hour incubation with S. aureus. Experiments are performed to confirm that the active domain degrades bacterial toxins and bacterial remnants and prevents bacterial induced apoptosis, and to confirm the bacterial induction of apoptosis macrophages.

Determination of Intracellular Location and Trafficking MMP-12

The data suggest that MMP-12 is contained in lysosomal granules, for release into phagosomes to form a phagolysosomes. Experiments are performed to determine the intracellular trafficking of MMP-12 at rest and during the stress state of bacterial infection. The location of MMP-12 is confirmed using colocalization to determine the intracellular compartments of MMP-12. MMP-12 is tracked using specific antibodies for MMP-12 and MMP-12 GFP fusion protein and antibodies for specific organelle markers, i.e. lysosome associated membrane glycoproteins (LAMP1 and LAMP2) (Research Diagnostics, Flanders, N.J.).

Peritoneal Macrophage Cell Cultures

Peritoneal macrophages are obtained for all experiments by the following method unless stated otherwise. Mice are injected with 6 ml of sterile Brewers thioglycoll media. Peritoneal macrophages are harvested by peritoneal lavage with 10 ml of iced normal saline instilled into the peritoneal cavity with a 21-gauge needle and withdrawn. Lavage is repeated for a total volume of 20 ml fluid. Peritoneal lavage fluid is then be centrifuged at 4° C. for 10 min at 600×g. Cells are resuspended in condition media (Dulbecco's Modified Eagles Media, 10% fetal bovine serum, Streptomycin 50 μg/ml, penicillin 50 μg/ml) centrifuged and washed twice as described above. Macrophages are plated in sterile 24-well CoStar plates in a concentration of 2.5×10⁵/well and washed at 1 hour and the following day to remove dead and non-adherent cells. This technique allows for cell cultures with >95% peritoneal macrophages determined by histological examination. On the day of the experiment, cells are washed ×3 in fresh condition media without antibiotics.

Intracellular Co-localization of MMP-12 and Bacteria

Peritoneal macrophages (5×10⁵ cells) are plated on Lab-Tek II Chamber Slide (Nalgene Nunc International, Rochester, N.Y.) 2 well chamber slides. Unchallenged macrophages are permeabilized with 100% methanol at −20° C. for 7 minutes. Cells are rinsed in PBS and primary antibody for MMP-12 diluted 1:250 in 2% fish gelatin solution (Sigma-Aldrich, St. Louis, Mo.) are added and incubated at 4° C. overnight. Goat anti-rabbit IgG FluoroLinkTMCyTM3 antibody (Amersham Biosciences, Piscataway, N.J.) is added the next day. Cells are rinsed with PBS and vectashield with DAPI Vector Laboratories, Burlingame, Calif.) mounting media is added. Intracellular MMP-12 location is assessed using fluorescent microscopy. For co-localization, the above-described technique is performed and antibody specific for lysosomal cell marker LAMP1 (Santa Cruz) with FITC labeled secondary antibody is added. Peritoneal macrophages infected with S. aureus and E. coli undergo similar staining techniques during bacterial infection with. Co-cultures are incubated at 37° C. in 5% CO₂ incubator. Bacteria are washed off after 10 minutes of incubation and an antibiotic media (penicillin 50 μg/ml, gentamicin 50 μg/ml) is added to kill extracellular and membrane bound bacteria. Co-cultures are stopped by the addition of iced sterile PBS and undergo permeabilization and fixation as described above. Co-culture consists of one, two, and four hour time points starting from the addition of bacteria. Macrophages are again stained for MMP-12 and lysosomal markers, LAMP1, LAMP2 and lysozyme. Other potential markers consist of pH-sensitive and calcium-sensitive probes (Molecular Probes, Eugene, Oreg.). Both types of probes further determine the intracellular conditions under which MMP-12 is localized. These experiments will identify the optimum intracellular conditions under which MMP-12 is active as an anti-microbial agent. For example the optimal pH for enzymatic activity of MMP-12 is 7.4 and lysosomes can attain a pH of 4, which is below the optimal pH for MMP-12 enzymatic activity (pH of 7.2). This further confirms a role for the enzymatic domain of MMP-12 against bacteria.

Determination of Subcellular Location of MMP-12 by Density Gradient Centrifugation

A second method for localization uses sub-cellular fractionation and density gradient centrifugation. Peritoneal macrophages from SvEv/129 mice are obtained as previously described herein. Peritoneal macrophages (2×10⁸) are incubated in Teflon coated wells (CoStar) and resuspendend in disruption buffer (100 mM KCl, 3 mM NaCl, 1 mM ATP, 3.5 mM MgCl₂ 10 mM PIPES, pH 7.2 and EGTA 1.25 mM and 0.5 mM phenylmethylsulfonyl fluoride). Macrophages are disrupted by nitrogen cavitation. Sub-cellular fractions are separated by density gradient centrifugation using Percoll gradient containing three layers of density of 1.05/1.09/1.12 g/ml and centrifuged at 37,000×g for 30 minutes. Sub-cellular compartments are screened for the presence of MMP-12 by western blot analysis. Controls for the sub-cellular fractions consist of MMP-12−/− peritoneal macrophages, which undergo similar procedure. Fractions that contain MMP-12 are screened for the presence of lysosomal associated proteins such as lysozyme and LAMPs using commercially available antibodies (Santa Cruz biotechnology, Inc., Santa Cruz, Calif.). Co-localization of other macrophage MMPs is determined by gelatin zymography on 10% SDS-PAGE containing 1 mg/ml gelatin on non-reducing conditions.

Determination of Intracellular MMP-12 under Real-time Conditions

A second set of experiments is performed to confirm the trafficking of MMP-12 under real-time conditions. A full-length MMP-12 fluorescent C-terminal tag fusion protein is generated in these experiments. DNA expression vector consists of pDsRed1-N1 vector (Clontech). The DsRed-MMP-12 expression vector is constructed by amplifying the coding region of the full-length mouse MMP-12 containing the endogenous signal peptide by PCR amplification. MMP-12 is ligated using BglII and SacII restriction sites, which generates a C-terminal DsRed fusion protein. The expression vector contains a CMV promoter and neomycin selection marker. This fusion protein generates a MMP-12 C-terminal red fluorescent fusion protein. MMP-12 DsRed expression vector is transfected into the P388 macrophage cell line (ATCC). Transfection uses FuGene 6 Transfection Reagent (Roche Molecular Biochemicals, Indianapolis, Ind.). Transient transfection experiments occur 24 hours after transfection. Stable cell lines are selected using 400 μg/ml of G418 (Gibco-BRL). After 10 days of selection, cells are cloned by limiting dilution. One cell line that shows good DsRed fluorescence is used for all experiments. MMP-12 red fluorescent fusion protein production is verified by western blot analysis using DsRed antibody (Clontech). For control, cells are transfected with DsRED vector lacking MMP-12 insert. Cells are grown on Lab-Tek chamber slides (Nalgene Nunc Int.) and observed using fluorescence microscopy (Carl Zeiss) with cooled CCD camera and Metamorph imaging software. Co-culture experiments using the MMP-12- DsRed expressing cell line follow previously described protocols using E. coli (DH-5α) transfected with EGFP expression vector (Clontech). Cells are visualized for bacterial uptake and co-localization of MMP-12 and intracellular bacteria. Results from these studies are used to confirm real-time co-localization of bacteria and MMP-12.

A second benefit of this system is the location of the GFP tag on the c-terminal domain. Previous intracellular localization of MMP-12, has used a polyclonal antibody to the active domain. The C-terminal has antimicrobial activity and it can be cleaved from the active domain through autolytic separation. Experiments will further confirm the amount of C-terminal that is attached to the full-length MMP-12 and C-terminal that is cleaved by lysing cells with weak detergent and confirm the forms of C-terminal domain by western blot analysis. Macrophages are co-incubated with bacteria for two hours. Cold incubation is stopped with iced PBS and cells lysed with triton 0.2%. Western blot analysis is performed using antibody to GFP. These results are compared to cell lines that are transfected with DsRed vector alone.

Determination of Susceptible Bacteria to Intracellular MMP-12

To confirm changes of intracellular killing capacity of macrophages lacking MMP-12 against a range of gram positive and gram negative bacteria, MMP-12−/− macrophages are challenged using the antibiotic protection assay described previously herein. Briefly, peritoneal macrophages from MMP-12−/− and MMP-12+/+ mice are co-incubated with bacteria in a 10:1 ratio. Macrophages are washed after one hour and an appropriate antibiotic media is added to kill extra-cellular and membrane-bound bacteria. Macrophages are washed and then lysed with triton 0.1% over a two-hour time course. Lysates are diluted in PBS and then plated on LB agar plates for 18-hour incubation. Bacteria CFU are counted and results are used to determine the intracellular quantity of bacteria. Bacterial strains consist of the types previously described herein: S. pneumonia, E. faecalis, E. coli (K1), H. influenzae.

Determination of Bacterial Induced MMP-12−/− Macrophage Death during Infection

Data of MMP-12−/− macrophage co-culture with S. aureus show signs of programmed cell death (PCD) by electron microscopy. Experiments to confirm that intracellular MMP-12 has a function in the prevention of bacterial induced PCD are performed. To determine cell death of MMP-12+/+ macrophages during bacterial infection, MMP-12−/− peritoneal macrophages are challenged with S. aureus and are assessed for PCD.

MMP-12−/− and MMP-12+/+macrophages are plated in Lab-Tek chamber slides (2×10⁵ cells/well) and cultured with S. aureus for two hours. Co-cultures are washed with PBS at 4° C. and macrophages are stained with Sytox Dead cell stain (Molecular Probes). Positive-staining cells are determined by fluorescent microscopy and quantified by counts/HPF. S. aureus in mid log phase of growth is added in 10-fold higher quantity. Cells are co-incubated in 5% CO₂ injected humidity incubator 37° C. Macrophage co-culture are stopped by the removal of cell suspension and centrifuged in sterile PBS 4° C. The experiment is performed in a 90-well plate. To confirm an increase in apoptotic macrophages during bacterial infection, co-culture undergoes the experimental conditions and undergoes TUNEL assay (Trevigen, Gaithersburg, Md.) to determine apoptotic. Positive cells are quantified by high-powered microscopy.

Determination of Bacterial Induced PCD in MMP-12−/− Peritoneal Macrophages

To confirm that S. aureus is inducing PCD experiments to determine whether macrophages are demonstrating signs consistent with PCD as well as changes in caspase levels are performed.

### Example 5

Determination of the Role of MMP-12 during Bacterial Pneumonia

The pneumonia model (described herein) showed that alveolar macrophages and MMP-12 play a significant function for cellular clearance of bacterial infection. Macrophages eradicate bacteria by phagocytosis and intracellular degradation. MMP-12−/− macrophages have impaired killing of ingested bacteria; eliminating an important cellular mechanism of initial host defense. Experiments are performed to confirm that MMP-12 has a role in in vivo antimicrobial activity against a range of bacterial pathogens. Macrophage's inability to degrade intracellular pathogens leads to cell death and the loss of its inflammatory orchestration. The intratracheal bacterial infection model system, is used to confirm the immunologic contributions macrophages during the initial period after bacterial infection and the role of MMP-12 in this setting for bacterial pneumonia. Experiments also confirm the efficacy of MMP-12 C-terminal as an antibiotic in setting of bacterial infection.

Determination of Bacterial Susceptibility of MMP-12−/− Mice

MMP-12 has a role in survival during S. aureus infections involving macrophage-rich environments. Experiments are performed to further define its significance of MMP-12 against a range of common pulmonary pathogens. Six MMP-12−/− mice and six wild type mice are intratacheally injected with Streptococcus pneumonia, Enterococcus faecalis, Escherica coli, and Haemophilus pneumoniae. After infection mice are monitored for decreased activity, weight loss and signs of respiratory distress. Mice are be euthanized and be defined as a mortality when signs of distress and inactivity or weight loss of >20% appear, in accordance with guidelines from the Brigham and Women's Hospital Department of Comparative Medicine. Varying doses of each bacterium are injected to determine differences in LD50 between MMP-12+/+ and MMP-12−/− mice. A difference of tenfold is defined as significant. Statistical analysis is used to determine significance in survival curves. To determine rate of bacterial clearance, sublethal doses of each organism are given and mice are euthanized at 2 and 24 hours as previously described above herein.

Determination of In Vivo Macrophage Death

In the pneumonia model system, macrophages after 2 hours showed intracellular proliferation. Experiments are performed to confirm the in vitro co-culture data that S. aureus are inducing macrophage death possibly through the induction of apoptosis as described above.

Define the Inflammatory Response during Infection in the Absence of MMP-12

Experiments are performed to confirm the differences in inflammation during S. aureus pneumonia in regards to inflammatory cell recruitment and activation. Groups of 4 mice each of MMP-12−/− and MMP-12+/+ are infected with intratracheal S. aureus. Mice are euthanized and lungs are removed and homogenized. A single-cell suspension is produced and stained with fluorescent antibodies against GRI for neutrophils, Mac3 for macrophage, CD3, CD4 and CD8 for lymphocytes, and NK1.0 for NK cells (Santa Cruz Biotechnology, Inc.). Lung tissue of infected mice is histologically examined to determine location of cellular components and to corroborate results from flow cytometry experiments.

The cellular content of bronchoalveolar lavage (BAL) from MMP-12−/− and MMP-12+/+ mice that are injected intratracheally with S. aureus is also examined. BAL is examined for cell count and cell differentiation. The production of cytokines released by alveolar macrophages, e.g., TNF-α, IL-12, GM-CSF is also assessed in the absence of MMP-12. Cytokine quantities from lung homogenates and BAL of MMP-12−/− and MMP-12+/+ mice infected with S. aureus are tested using ELISA plates (Genzyme Corp. Cambridge, Mass.).

These experiments are performed to confirm MMP-12 C-terminal improvement of survival in the setting of bacterial infection when used as an antibiotic. Recombinant murine MMP-12 C-terminal produced as described herein also is used. Mice undergo peritoneal infection with S. aureus and E.coli as described above herein. Mice receive sublethal doses of bacteria. MMP-12 CAMP is injected into the intraperitoneal space in a concentration of 50 μg/ml. Mice undergo peritoneal lavage to determine differences in bacterial load compared to wild type.

### Example 6

Introduction

We have identified MMP-12 as the first MMP with direct antimicrobial activity against Gram positive and Gram negative bacteria. Furthermore we have shown that MMP-12 has a novel intracellular and non-catalytic mechanism contained in its c-terminal hemopexin domain. These results reclassify MMP-12, a pathological matrix destructive proteinase, as an antimicrobial protein with importance for macrophage bactericidal activity and significant implications at the animal level.

A second thrust of these studies illustrate the enhance the role macrophages have during the early events after bacterial invasion. Macrophages, a tissue-fixed monocytic derived immune cell, serves as a sentinel in early host defense response against invading microorganisms. Macrophages' intracellular clearance mechanism is a multi stage process of phagocytosis, intracellular sequestration and degradation by reactive oxygen intermediates and proteolytic enzymes. Depending on the pathogen load and virulence, macrophages can further clear pathogens by recruiting accessory host defense cells such as neutrophils and in later stages, macrophages. Although macrophages have antimicrobial capability, bacterial clearance has long been thought to be primarily the function of neutrophils, and it was believed that macrophages are limited to later stages of bacterial removal and clearance of proteinaceous inflammatory debris. Despite our current understanding of the macrophage, its overall contribution to the clearance of bacterial invasion has not been fully defined. Our results have clarified the role of macrophages play during the early phase of bacterial invasion and the results when impaired macrophage are deficient in host response effector mechanism.

Methods

Mice: MMP-12−/− mice were previously generated as described above herein, and were maintained in the 129/SvEv background. MMP-12+/+ mice were littermates. All mice were housed in pathogen free barrier facility and studied under procedures approved by the Institutional Animal Care and Use Committee. Adult mice ages >12 weeks were used for these experiments and matched for age and sex.

Bacteria: S. aureus a clinical isolate and E. coli (K1) were used in these experiments, as described above herein. Bacteria were grown in tryptic soy broth (Difco, Detroit, Mich.) for 18 h at 37° C. Bacteria in mid log phase growth were centrifuged washed in sterile phosphate buffered saline (PBS). Concentration of bacteria was determined with absorbance at 540 nm. A standard of absorbencies based on known CFU was used to calculate the inoculum concentration. Quantity was confirmed by dilution and next day CFU count.

Peritonitis model: Mice received intraperitoneal injection of bacteria in a total volume of 6 ml. Mice were observed over a 72 hour period for signs of distress and mortality. Mice demonstrating signs of respiratory difficulty or distress were euthanized. Mortality was recorded.

Hematogenous model: MMP-12+/+ and MMP-12−/− mice were anesethized using 2.5% avertin. S. aureus (1×10⁸ CFU) in 400 μl of PBS was injected via tail vein. Mice were observed daily over a two week time period for signs of distress and mortality. A second group of mice (n=12 for each group) were hematogenously injected with S. aureus (1×10⁶ CFU). Mice were euthanized at 2 and 24 hours. Lungs were flushed with one ml of sterile saline and removed aseptically. Left lung, kidney, and spleen were homogenized with a tissue homogenizer under a vented hood. Homogenates were placed on ice, and diluted. Aliquots were plated on LB agar plates (Dilfco) and incubated for 18 h at 37° C. for CFU count.

Pneumonia model: MMP-12−/− and MMP-12+/+ mice were anesthetized with 2.5% avertin. The trachea was exposed through an anterior midline incision using sterile technique. S. aureus was injected 100 μl volume using a 30-gauge needle. Injection site was left opened and mice were observed daily for signs of distress. To assess bacterial load at 2 h or 24 h (S.aureus) post infection, MMP-12−/− and MMP-12+/+ mice (n=12) received an intratracheal injection of S. aureus (1×10⁶ CFU). Mice were euthanized by CO₂ asphyxiation, left lung was removed and homogenized in sterile PBS. Serial dilutions of homogenates were plated on LB plates and incubated at 37° C. for 18 hours and CFU count. Right lung was inflated to 25 cm H₂ with 10% buffered formalin for paraffin embedding.

Histology: Tissues were perfused, inflated (for lung only), fixed in 10% buffered formalin, and processed for paraffin sections. Routinely, 5-micron paraffin sections were cut and stained with hematoxylin and eosin (H&E) and brown and brenn bacterial stain.

Peritoneal Macrophages: Mice of each genotype were injected with 1 ml of sterile Brewers thioglycoll media. After 3 days peritoneal cavity was lavaged with 10 ml (×2) of 0.9% saline. Lavage fluid was centrifuged, washed and resuspended in condition media (Dulbecco's Modified Eagles Media, 10% fetal bovine serum, streptomycin 50 μg/ml, penicillin 50 μmg/ml). Cells were seeded in 24 well plate (Costar) in concentration of 2.5×10⁵ macrophages/well and washed after 10 min to remove dead and non-adherent cells. Verification of macrophage purity was determined by cytospin and staining of suspension (Diff-Quik Stain set; Dade Behring, Newark, Del.) for differential cell counts using a high-power microscope. On the day of experiment, cells were washed and antibiotic-free media was added.

Macrophage Intracellular Killing: S. aureus was added at a concentration of 10 bacterium per macrophage. Co-cultures were incubated at 37° humidified in a 5% (vol/vol) CO₂ injected incubator for one hour. Co-cultures were washed with sterile PBS×3 and condition media was added containing appropriate antibiotics (100 μg/ml gentamicin, 100 μg/ml penicillin). Cultures were incubated for 30 minutes to kill extracellular and membrane bound bacteria (time0). At each time point condition media was removed, cells were washed and permeabilized with 200 μl of sterile 0.2% triton PBS solution. Cell lysates were diluted in sterile PBS and plated on LB agar plates and incubated 18 hours at 37° C. for CFU count.

Electron microscopy: Peritoneal macrophages (2×10⁶) were cultured in Teflon-coated wells (Costar) in antibiotic free condition media. Staph aureus (6×10⁶ CFU) added for two hour incubation. Co-culture was stopped and cells were fixed with iced 5% glutaraldehyde solution for processing electron microscopy.

Bacterial Expression and Purification of Recombinant MMP-12 C-terminal

MMP-12 C-terminal cDNA was ligated as an EcoRV/EcoRI cassette ubti te pET 20 b vector which permitted translation in the proper reading frame beginning with amino acid 269 to 462 and including 6×histidine C-terminal tag. pET 20b alone and pET 20b/MMP-12 C-terminal were transformed into the E.coli strain BL2(DE3)LysE(Novagen Inc.). Protein was resuspended in 6 M urea 300 mM NaCl, 50 mM NaPO₄ pH 8.0 and purified using Talon binding resin (Clontech). Recombinant protein was dialyzed slowly using against 50 mM sodium phosphate 300 mM NaCl 0.75 M Urea pH 7.4 buffer. Recombinant protein identity was verified by Western blotting using antibody to 6×histidine residue (Invitrogen). Concentration was determined using Bradford colorimetric assay. Coomassie-stained 10% PAGE demonstrated single band without contaminating proteins.

Reagents

Activated MMPs Human MMP 3 (cc1035) MT1-MMP (CC1041), Matrilysin (CC1059), MMP-13 (CC068) MMP-2 (CC071) were obtained from Chemicon. Peptides were obtained from Genemed Synthesis Inc. with >95% purity. The peptides used included: MMPAP-12 C-terminal peptide I: SRNQLFLFKDEKYWLINNLV (SEQ ID NO:37; 333-352 a.a.), MMPAP-12 peptide II: RSIYSLGFSASVKKVDAAVF (SEQ ID NO:40; 359-378 a.a.) and MMP-13 peptide: SRDLMFIFRGRKFWALNGYD (SEQ ID NO:40; 343-302 a.a.). Peptides were solubilized in Milli-Q purified H₂O.

In Vitro antimicrobial activity: E.coli, and S aureus were grown in TSB at 37° C. and washed twice with PBS. Mid-log phase bacteria (10⁵) were incubated in the absence or presence of purified MMP-12 C-terminal in a total volume of 100 μl of 10 mmol/L sodium phosphate containing 5% (vol/vol) TSB at 37° C. for 1 hour. Serial dilutions were then spread on agarose plates and the number of CFUs were determined after overnight incubation.

Direct bactericidal Assay: E. coli and S. aureus were incubated in the presence of MMP-12 C-terminal for one hour at 37° C. Fluorescent probes Syto 59 and S-7020 (Molecular Probes) were added for a final concentration of 5 μM and 20 μm respectively and incubated at room temperature for 5 minutes. Bacterial cultures were 20 μl aliquot was placed on glass slide and directly visualized. Images were obtained using digital Spot camera at 200× magnification. Quantification of dead versus total cells was performed using Metamorph image analysis software.

Bacterial membrane vesicle: S. aureus, grown to midlog phase of growth, centrifuged and the pellet was freeze fractured using dry ice. Chloroform/methanol (2/l) was added to a final volume of 5 ml. Mixture was agitated for 20 min in an orbital shaker at room temperature. Suspension was centrifuged (2000 rpm) and the lipid phase was removed. Chloroform was evaporated under vacuum. Bacterial membrane lipids were hydrated in a 1 mM CaCl, 10 mM MOPS 100 mM KCl pH 7.2. Bacterial membranes were freeze fractured and incubated in the presence of fluorescent Calcium Green™-1Dextran conjugates 3000 MW (Molecular Probes). Bacterial membrane vesicles were incubated in the presence and absence of MMP-12 C-terminal protein, 20 μg/ml for one hour. Fluorescent membrane vesicles were visualized using Nikon microscope 200×magnification. Images were captured using Spot camera.

Statistical Analysis: Experiments were performed in triplicates. Standard deviations of the means were determined. All tabulated or illustrated values were representations of at least 4 separate experiments. Significant differences between means were determined by Student's T-test. A P-value of <0.05 was considered significant.

Results

MMP-12−/− Mice have Increased Mortality during Bacterial Peritonitis

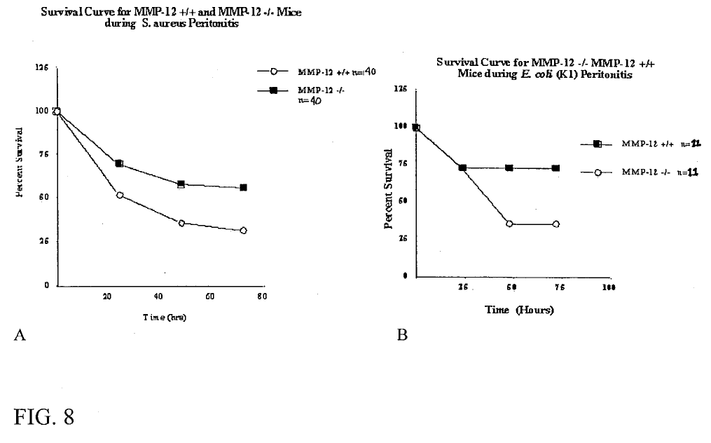

To test for a function of MMP-12 in host defense, MMP-12−/− mice and wild type littermates (MMP-12+/+) received infectious challenges to macrophage rich environments using a prototypical Gram positive bacterium, S. aureus. MMP-12−/− and MMP-12+/+ mice received an intraperitoneal inoculation of S. aureus (4×10⁸ CFU) and followed for 72 hours. MMP-12−/− mice demonstrated clinical signs of sepsis consisting of decreased activity ruffled fur and labored respiration with a mortality rate of 100% compared to 72% for MMP-12−/− mice after 72 hours. Mice were then challenged with a Gram negative bacteria, Escherica coli (K1) (1×10⁸ CFU), a more typical peritoneal pathogen. Similar to S. aureus, MMP-12−/− mice had increased susceptibility to E. coli peritonitis compared to MMP-12+/+ mice. MMP-12−/− and MMP-12 +/+ mice had mortality rate after 72 hours of 10% versus 40% respectively. These results demonstrated a novel function for MMP-12 for the improvement of survival during gram-positive and gram-negative bacterial peritonitis. Additional trials were performed as described with 40 mice for S. aureus peritonitis and 10 mice for E. coli (K1) peritonitis and the results are illustrated in FIGS. 8 A and B respectively. In each case the MMP-12 +/+ mice had a lower mortality rate than their MMP-12−/− counterparts.

MMP-12−/− Mice have Increased Mortality during S. aureus Pneumonia but not Hematogenous Infection

Results from the peritonitis experiments demonstrated a role for MMP-12 in the setting of infection. Since the peritoneum contains macrophages as a first line of host defense, it stood to reason that other macrophage containing organs, such as the lung, would demonstrate similar dependence on MMP-12 for survival during bacterial challenge. To test this hypothesis, S. aureus (1×10⁸ CFU) was instilled into the pulmonary parenchyma via intratracheal injection. MMP-12−/− mice again showed signs of bacterial sepsis, as previously described, while MMP-12 +/+ mice demonstrated fewer and milder symptoms to the challenge. Survival differences for the two strains of mice revealed a two week mortality rate of 44% for MMP-12−/− mice with the majority of deaths occurring during the first 48 hours compared to a 19% mortality rate for MMP-12+/+ mice.

To further define the impact of MMP-12 during a systemic infection, we inoculated mice hematogenously with S. aureus (4×10⁸ CFU). Survival rates for two weeks did not reveal differences between MMP-12−/− and MMP-12+/+ mice with both groups of mice having a mortality rate of 62%. Result from the hematogenous survival suggested that MMP-12, although improving survival during peritonitis and pneumonia, does not exerts its host defense activity when bacteria circumvent macrophages.

MMP-12+/+ Mice have Impaired Pulmonary Clearance of Bacteria

We postulated from our in vivo experiments that MMP-12 deficiency contributed to murine death during bacterial infection due to an impaired macrophage clearance of bacteria. We first tested for the requirement of macrophages and MMP-12 in the clearance of bacteria in organs with varying quantities of tissue macrophages. We hypothesized that MMP-12 had a regional clearance of bacteria based on the presence of tissue macrophages and not due to a systemic response such as the release of pro-inflammatory cytokines. MMP-12−/− and MMP-12+/+ mice were hematogenously infected (n=12 each group) with a sub-lethal dose of S. aureus (1×10⁶ CFU). Mice were euthanized at 2 and 24 hours for harvesting of spleen, kidney and lung. Tissues were homogenized and diluted for CFU count. Results from this experiment demonstrated similar bacterial burden in spleen and kidney at both 2 and 24 hours for both groups of mice. Lung cultures revealed a larger bacterial load at 2 hours and by 24 hours had a 5 fold more bacteria than MMP-12+/+ mice. MMP-12+/+ mice had lower levels at both 2 and 24 hours with a trend toward bacterial clearance. From this data, we determined that although MMP-12 did not affect survival during hematogenous infection, it served a function in the bacterial clearance of infection from macrophage rich region of the lung.

To further determine pulmonary dependence on macrophage and MMP-12 to clear bacteria, we challenged MMP-12−/− and MMP-12+/+ (n=12 each group) mice with an intratracheal sub-lethal dose of S. aureus (6×10⁷ CFU). Lungs were harvested at 2 and 24 hours, similar to the hematogenous challenge. The results of this experiment demonstrated a larger bacterial load in the lungs of MMP-12−/− mice at 2 hours with a 10-fold increase in bacteria compared to MMP-12+/+ mouse lungs. At the 24 hour time point both groups of mice were able to clear bacteria, potentially through the use of secondary inducible bactericidal mechanisms. Lung histology from the groups of mice did not show any significant difference in neutrophil numbers or macrophages at either 2 or 24 hours after the inoculation (FIG. 4E). Lung tissue stained for bacteria demonstrated bacteria were concentrated inside alveolar macrophages in the MMP-12−/− mice at the two hour time point and not in the MMP-12+/+ mice lungs consistent with our CFU counts. These experiments demonstrated that MMP-12 had a role in bacterial clearance from a macrophage containing organ and was localized to alveolar macrophage intracellular killing and not to neutrophil recruitment.

MMP-12 is Required for Intracellular Macrophage Anti-microbial Activity

Lung histology suggested a role for intracellular MMP-12 in the clearance of bacteria during invasion into the distal parenchyma. We tested for an intracelluar macrophage bacterial killing function for MMP-12 by co-culturing peritoneal macrophages from MMP-12−/− and MMP-12+/+ mice with S. aureus using an antibiotic protection assay. Prior to the addition of bacteria, peritoneal macrophages were washed several times prior to remove extracellular MMP-12. Bacteria were then co-incubated for one hour to allow for adequate phagocytosis. Co-cultures were washed with PBS and an antibiotic media (gentamicin 100 μg/ml, penicillin 100 μg/ml) was added to kill extra-cellular and membrane bound bacteria. Over a 90 minute time course, macrophages were permeabilized with triton 0.2% and lysates were diluted and plated on LB agar plates for overnight incubation and next day CFU count. Bacterial counts were then used as a representation of total viable intracellular bacteria. Results from these experiments revealed MMP-12−/− macrophages had 10 times more intracellular bacteria than wild-type control (FIG. 4.) after a 90 minute time course. These findings have been repeated (n=6) with the consistent finding of impaired antimicrobial function of MMP-12−/− macrophages. Electron microscopy of the peritoneal macrophages co-incubated with S. aureus two hours revealed intracellular proliferation of bacteria in MMP-12−/− macrophages along with signs of cell death. MMP-12 macrophages had significantly fewer bacteria (FIG. 5). Findings from both intracellular killing experiments and electron microscopy reveal a novel and unreported intracellular anti-microbial activity of MMP-12 unique amongst the MMP family.

MMP-12 C-terminal has In Vitro Antimicrobial Activity

Since recombinant MMP-12 demonstrated a non-enzymatic in vitro antimicrobial effect, we further attempted to isolate the region of antimicrobial activity by generating recombinant protein of the 26 kDa C-terminal domain. Recombinant C-terminal domain was co-incubated with S. aureus and showed similar activity and dose response as the full length MMP-12 with a 90% antimicrobial activity at 20 μg/ml. Recombinant C-terminal domains of MMP-2 and MMP-9 were also generated to test for the novelty of MMP-12 C-terminal (a MMPAP-12 polypeptide) antimicrobial function. When incubated under similar conditions only MMP-12 C-terminal domain demonstrated antimicrobial effects.

MMP-12 Kills Bacteria by Disrupting Bacterial Membrane

To determine the mechanism of action for the antimicrobial activity of MMP-12, we postulated that MMP-12 has similar activity against bacteria as other recently described antimicrobial peptides in the disruption of the bacterial membrane. In order to determine the ability of MMP-12 to disrupt the bacterial membrane we co-incubated S. aureus with the MMP-12 C-terminal and added a hydrophilic fluorescent dye that is able to penetrate bacteria after disruption of the cell wall. Bacteria that developed cell leakage will fluoresce while intact bacteria will not. Results of these experiments revealed that bacteria that were incubated with MMP-12 C-terminal developed cell membrane leakage after one hour while bacteria incubated with control media did not show loss of fluorescence. To further verify that MMP-12 C-terminal was directly causing membrane damage, bacterial membrane vesicles from S. aureus cell wall were generated and loaded with a 3000 MW fluorescent dextran. MMP-12 C-terminal (20 μg/ml) and control media were incubated with the membrane vesicles for thirty minutes. An aliquot of co-culture was placed on a slide for visualization with fluorescent microscopy. Results from these experiments revealed loss of vesicle fluorescence compared to control. Signifying MMP-12 C-terminal directly permeabilized the vesicle membrane allowing for extravasation of dextran.

MMP-12 Contains a Conserved Amino Acid Sequence with Antimicrobial Activity Antimicrobial

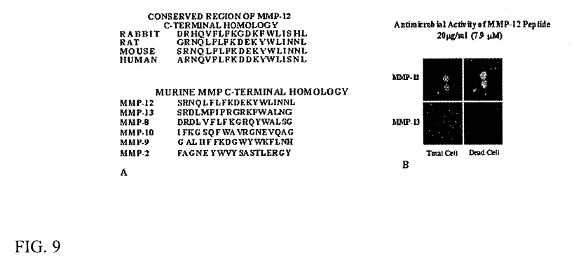

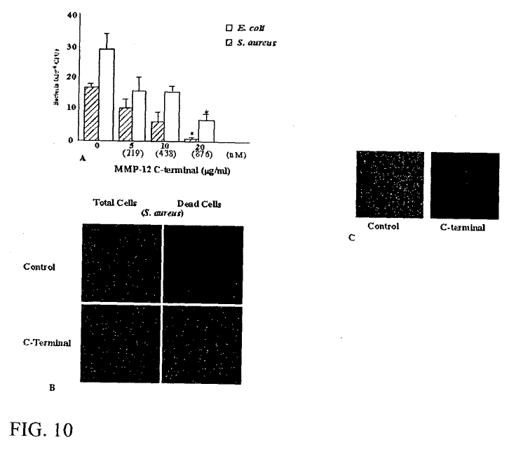

Recombinant segments of MMP-12 C-terminal were generated each covering one third of the C-terminal. Segments were then tested for anti-microbial activity against S. aureus. The second segment demonstrated antimicrobial effect while the first and third regions showed little effect. We hypothesized that in this region there was a secondary structure that had potential antimicrobial structure and properties consistent with the structure in cathelicidins. A predicted amphipathic and alpha helical structure was found in this region, which was conserved in the MMP-12 C-terminal domains from rabbit, rat, mouse and human. This region was unique when compared to other members of the MMP family shown in FIG. 9A. To determine if this region contained antimicrobial properties peptides were generated of the murine MMP-12 region (SRNQLFLFKDEKYWLINNLV (SEQ ID NO:37; 333-352 a.a.), and homologous region in MMP-13 peptide (SRDLMFIFRGRKFWALNGYD (SEQ ID NO:40; 343-302 a.a.)) for control. MMP-12 and MMP-13 peptides (20 μg/ml) were incubated with S. aureus for 30 minutes. Bacterial death was determined using propidium iodide exclusion assay and visualized with fluorescence microscopy. FIG. 9B illustrates our results, which revealed bacteria incubated in the presence of MMP-12 peptide had clumping and increased uptake of membrane impermeant dye compared to bacteria incubated with MMP-13 which had little dye uptake. These studies have been repeated n>10 with similar results. FIG. 10 illustrates thae effect of MMP-12 C-terminal domain on cell survival.

MMP-12 is the only MMP to have Direct Antimicrobial Activity

To test for direct activity, functional full length recombinant human MMP-12 was incubated with S. aureus in a 5% LB culture. Commercially available full length pro-MMPs 2,3,7,8, and 9 tested under similar conditions did not demonstrate this direct antimicrobial activity. MMP-12 enzymatic activity was not required for this antimicrobial effect. Full-length MMP-12 was inhibited under different conditions either with hydroxamic acid, an irreversible MMP inhibitor. Furthermore, rMMP-12 active domain alone did not kill bacteria at similar doses and conditions. These studies demonstrated pro-MMP-12 had a direct anti-microbial effect not dependent on its enzymatic activity.

Discussion

MMP 12 as an Antimicrobial Peptide

MMP-12 is a 54 kDa protein that consists of three separate domains. During the process of activation, MMP-12 undergoes cleavage of the amino terminal domain for activation of the enzymatic domain. It further undergoes the cleavage of the C-terminal domain by what is postulated to be an autolytic event. The processing of the C-terminal has been thought to be more representative of MMP-12's potent enzymatic activity and not the release of a functioning protein. Furthermore MMP-12's C-terminal has not been ascribed to having any physiological function. Our studies have further determined that MMPAP-12 has activity against both gram positive and gram negative bacteria. Similar to other antibacterial peptides, defensins and cathelicidins, disrupts the bacterial cell wall causing bacterial death. This antimicrobial effect is unique to MMP-12 and the from the other members of the MMP family. C-terminal antimicrobial activity involves in a 22 amino acid. region (SEQ ID NO: 42 is the human 22 amino acid C-terminal MMPAP-12 and is encoded by SEQ ID NO:44, (SEQ ID NO: 43 is the mouse 22 amino acid C-terminal MMPAP-12 and is encoded by SEQ ID NO:45). This sequence contains a predictive amphipathic and alpha helical structure. Amino acid sequence is unique from other members of the MMP family and is unique from other members of the antimicrobial peptides. Cellular activity. Macrophages are the primary source of MMP-12 Macrophages provide a first line cellular host defense against microbial invasion. Macrophage clearance of bacteria depends on phagocytosis and intracellular degradation. MMP-12 is produced almost exclusively by macrophages and stored in granules at rest. Our studies for the first time link macrophage antimicrobial activity and intracellular stores of MMP-12.

Stored MMP-12 represents a pool of antimicrobial peptides. During the process of bacterial recognition and phagocytosis, bacteria are attacked by MMP-12. Killing of bacteri a occurs in a rapid fashion during the first two hours after ingestion. MMP-12 has similar physiological properties to the other antimicrobial peptides. The MMP-12 carboxy terminal domain contains stretches of amino acid sequences that have predicted amphipathic alpha helical structure. Pore formation of bacterial cell wall induces lysis of bacteria. In the absence of MMP-12, macrophages lack this important mechanism of bacterial degradation. During this crucial time period after phagocytosis, bacteria intracellularly proliferate. These experiments also demonstrated a novel intracellular antimicrobial activity not previously demonstrated in other MMPS. Activity appears to be non-enzymatic and is located with in the carboxy terminal domain.

In vivo model systems showed that MMP-12 is important for host defense against gram positive bacterial infections. Current understanding of this enzyme has been associated with its role in matrix destructive disease states. Lungs contain alveolar macrophages and maintain a sterile environment. Loss of this clearance mechanism has large impact on survival in initial macrophage infections.

These studies reiterated the importance of macrophage function in the clearance of microbial invasion. Macrophages are active during the initial stage of infection. After two hours MMP-12−/− macrophages were overwhelmed by the intratracheally induced S. aureus. Mortality for these mice were higher than compared to control in both pneumonia model and in intraperitoneal infection. Mortality was seen after a relatively short period again suggesting that the events occurring with in the initial stage of infection have ramifications toward survival. Most likely this represents a threshold of bacterial burden. With the loss of a macrophage antimicrobial defense, bacteria are able to proliferate and subsequently overwhelming subsequent host defense mechanisms. Macrophages and MMP-12, therefore acts as a central innate immune effector function for the lung and the peritoneum.

MMP-12 has a novel function in the clearance of bacteria. This data shows a physiological function for the clearance of bacteria by macrophages. MMPs extracellular function in the degradation of matrix protein is well described. Antibiotic protection assay for the MMP-12−/− and wild type peritoneal macrophages, illustrated intracellular function. Lack of MMP-12, gives phagocytized bacteria an intracellular survival advantage over bacteria. S. aureus was able to proliferate inside a phagosome. This suggests that intracellular MMP-12 has a role in the intracellular degradation. Either an indirect via cleavage of pro-forms of other antimicrobial peptides like lysozymes or directly degrading the bacterial cell wall. An alternative direct function is in the ability of a linear peptide domain that has pore forming capabilities.

### Example 7

Methods

Bacterial preparation: Staphylococcus aureus a clinical isolate was grown in tryptic soy broth for 18 hours at 37° C. An aliquot was placed in fresh media and grown until mid-log phase of growth. S. aureus was centrifuged and washed in sterile PBS and diluted. Bacteria concentration of O.D.₅₄₀ of 0.9, corresponding to a concentration of 6×10⁷ CFU/ml was used for inoculation.

Peptide preparation: Murine MMP-12 C-terminal peptide SRNQLFLFKDEKYWLINNLV (SEQ ID NO:37; 333-352) and Human MMP-12 C-terminal peptide ARNQVFLFKDDKYWLISNLR (SEQ ID NO:36; 341-359) and a human MMP-12 peptide with a single nucleotide polymorphism: ARNQVFLFKDDKYWLISSLR (SEQ ID NO:55) were solubilized in sterile H₂O at a concentration of 4 mg/ml.

Peritonitis model: C57/B16 mice received intraperitoneal injection of bacteria in a total volume of 1 ml (6×10⁷ CFU). Peptides were intraperitoneally injected immediately after at a dose of 1 mg. Mice were observed for signs of distress and mortality (see Methods described above herein). The control mice received the same intraperitoneal injection of bacteria as in the test groups and then received an injection of vehicle with no peptide.

Dose response: samples of S aureus were incubated with various concentrations of murine peptide (SEQ ID NO: 37), human peptide (SEQ ID NO: 36) and Human SNP peptide (SEQ ID NO:55). The human SNP peptide (SEQ ID NO:55) has a single nucleotide change from the sequence of SEQ ID NO 36. The peptide SEQ ID NO:36 has the amino acid sequence: ARNQVFLFKDDKYWLISNLR and the peptide SEQ ID NO:55 has the amino acid sequence ARNQVFLFKDDKYWLISSLR. The amount of bacteria remaining at various the various concentrations was determined for each group of a 100 minute time course.

Results

Preliminary Results at 72 Hour Time Point

Test and control animals were observed at 72 hour time point. Control mice (n=3) clinically mice demonstrated decreased activity and have 100% survival. Murine MMP-12 peptide mice (n=4) demonstrate decrease activity and have 75% survival. Human MMP-12 peptide mice (n=4) demonstrate normal activity and have 100% survival.

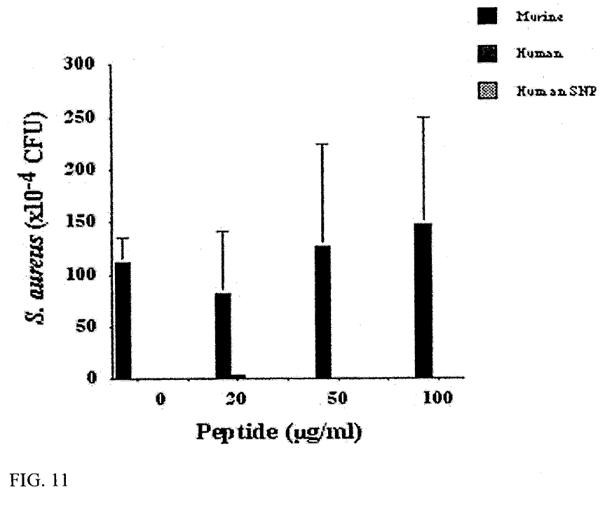

FIG. 11 illustrates the response of S.aureus to various doses of MMP-12 C-terminal peptides. The human peptide (SEQ ID NO:36) had zero S. aureus at most concentrations of the peptide. The Human SNP (SEQ ID NO:55) had zero S aureus at all concentrations and the response to the murine peptide (SEQ ID NO:37) was higher at each concentration of peptide. 100 minutes.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications as of the invention in addition to those shown and described herein will become apparent to those skilled in the art from the foregoing description and fall within the scope of the appended claims. The advantages and objects of the invention are not necessarily encompassed by each embodiment of the invention. It is understood that any mechanism of action described herein for the MMPAP-12 polypeptides is exemplary only and is not intended to be limiting, and the scope of the invention is not bound by any mechanistic descriptions provided herein.

All references, patents and patent publication that are recited in this application are incorporated in their entirety therein by reference.

We claim:

1. An isolated MMPAP-12 polypeptide molecule, wherein the MMPAP-12 polypeptide molecule does not have the amino acid sequence set forth as SEQ ID NO:13 or SEQ ID NO: 15.

2. The isolated MMPAP-12 polypeptide molecule of claim 1, wherein the polypeptide molecule is selected from the group consisting of SEQ ID NOs:1-6, 36, 37, 42, and 43 and functional homologs thereof.

3. A therapeutic composition comprising the isolated MMPAP-12 polypeptide molecule of claim 1, in a pharmaceutically acceptable carrier.

4. A method for treating or preventing an infection in a subject having or at risk of developing the infection, comprising
administering to a subject in need of such treatment a therapeutically effective amount of an MMPAP-12 polypeptide molecule, or functional homolog thereof for treating or preventing the infection.

5. The method of claim 4, wherein the MMPAP-12 polypeptide molecule is selected from the group consisting of SEQ ID NOs:1-6, 36, 37, 42, and 43.

6. The method of claim 4, wherein the infection is a bacterial infection.

7. The method of claim 4, wherein the subject is a vertebrate.

8. The method of claim 4, wherein the subject is human.

9. The method of claim 4, wherein the polypeptide molecule is administered systemically.

10. The method of claim 4, wherein the polypeptide molecule is administered topically.

11. A method for treating or preventing an infection in a subject having or at risk of developing the infection, comprising
administering to a subject in need of such treatment a therapeutically effective amount of an MMPAP-12 nucleic acid molecule, or functional homolog thereof, for treating or preventing the infection.

12. The method of claim 11, wherein the MMPAP-12 nucleic acid molecule is selected from the group consisting of SEQ ID NOs:7-12, 38, 39, 44, and 45.

13. The method of claim 11, wherein the infection is a bacterial infection.

14. The method of claim 11, wherein the subject is a vertebrate.

15. The method of claim 11, wherein the subject is human.

16. The method of claim 11, wherein the nucleic acid molecule is administered systemically.

17. The method of claim 11, wherein the nucleic acid molecule is administered topically.

18. An isolated nucleic acid molecule that encodes the isolated polypeptide of claim 1, wherein the nucleic acid molecule does not have a nucleotide sequence selected from the group consisting of SEQ ID NO:14 and SEQ ID NO:16.

19. A therapeutic composition comprising the isolated nucleic acid molecule of claim 18, in a pharmaceutically acceptable carrier.

20. An expression vector comprising the isolated nucleic acid molecule of claim 18 operably linked to a promoter.

21. A host cell transformed or transfected with the expression vector of claim 20.

22. A transgenic non-human animal comprising the expression vector of claim 20.

23. A transgenic non-human animal of claim 22, that expresses a variable level of an MMPAP-12 molecule.

24. A method for producing an MMPAP-12 polypeptide molecule comprising providing an isolated MMPAP-12 nucleic acid molecule operably linked to a promoter, wherein the MMPAP-12 nucleic acid molecule encodes the MMPAP-12 polypeptide molecule or a fragment thereof, and
expressing the MMPAP-12 nucleic acid molecule in an expression system.

25. The method of claim 24, further comprising:
isolating the MMPAP-12 polypeptide or fragment thereof from the expression system.

26. The method of claim 25, wherein the MMPAP-12 nucleic acid molecule is selected from the group consisting of SEQ ID NOs:7-12, 38, 39, 44, and 45.

27. A kit comprising:
at least one container housing an MMPAP-12 polypeptide molecule of claim 1, and
instructions for administration of the polypeptide.

28. The kit of claim 27, wherein the MMPAP-12 polypeptide molecule, comprises an amino acid sequence selected from the group consisting of SEQ ID NOs. 1-6, 36, 37, 42, and 43.

29. A kit comprising:
at least one container housing an MMPAP-12 nucleic acid molecule of claim 18, and instructions for administration of the nucleic acid.

30. The kit of claim 29, wherein, the MMPAP-12 nucleic acid molecule comprises a nucleotide sequence selected from the group consisting of SEQ ID NOs:7-12, 38, 39, 44, and 45.

31. An anti-microbial composition comprising:
the polypeptide of claim 1 in contact with a surface of a material or mixed with a suitable material.

32. The anti-microbial composition of claim 31, wherein the material is selected from the group consisting of: food, liquid, an instrument, a bead, a film, a monofilament, an unwoven fabric, sponge, cloth, a knitted fabric, a short fiber, a tube, a hollow fiber, an artificial organ, a catheter, a suture, a membrane, a bandage, and gauze.

33. The anti-microbial composition of claim 31, wherein the anti-microbial is an anti-bacterial.

34. A method of preventing or treating microbial contamination of a material comprising,
contacting the material with an MMPAP-12 polypeptide in an effective amount to prevent or reduce the level of microbial contamination of the material.

35. The method of claim 34, wherein the MMPAP-12 polypeptide comprises an amino acid sequence selected from the group consisting of SEQ ID NOs:1-6, 36, 37, 42, and 43, and functional homologs thereof.

36. The method of claim 34, wherein the microbial contamination is bacterial contamination.

37. The method of claim 34, wherein the material is aqueous.

38. The method of claim 37, wherein the material is drinking water.

39. The method of claim 34, wherein the material comprises blood, a body effusion, tissue, or cell.

40. The method of claim 34, wherein the material is food.

41. A method for preparing an animal model of a disorder characterized by aberrant expression of an MMPAP-12 molecule, comprising:
administering to a non-human subject an effective amount of an antisense, siRNA, or RNAi molecule to an MMPAP-12 nucleic acid molecule to reduce expression of the MMPAP-12 nucleic acid molecule in the non-human subject.

42. A method for preparing a non-human animal model of a disorder characterized by aberrant expression of an MMPAP-12 molecule, comprising
administering to a non-human subject an effective amount of a binding polypeptide to an MMPAP-12 polypeptide to reduce expression of the MMPAP-12 polypeptide in the non-human subject.

43. The method of claim 42, wherein the binding polypeptide is an antibody or an antigen-binding fragment thereof.

44. The method of claim 43, wherein the antibodies or antigen-binding fragments are labeled with one or more cytotoxic agents

45. An antisense molecule, comprising a sequence that binds with high stringency to an MMPAP-12 nucleic acid but does not bind to a nucleic acid that encodes a protease domain of an MMP-12 nucleic acid.

46. The antisense molecule of claim 45, wherein the antisense binds to an MMPAP-12 nucleic acid selected from the group consisting of SEQ ID NOs:7-12, 38, 39, 44, and 45.

47. A kit for preparing a non-human animal model of a MMPAP-12-associated disorder in a subject comprising:
one or more of the antisense molecules of claim 46, and
instructions for the use of the antisense molecule in the preparation of a non-human animal model of a disorder associated with aberrant expression of an MMPAP-12 molecule


SEQUENCE LISTING (55 sequences)

SEQ ID NO: 1 - protein, 192 aa, Homo sapiens
PALCDPNLSFDAVTTVGNKIFFFKDRFFWLKVSERPKTSVNLISSLWPTLPSGIEAAYEIEARNQVFLFKDDKYWLISNLRPEPNYPKSIHSFGFPNFVKKIDAAVFNPRFYRTYFFVDNQYWRYDERRQMMDPGYPKLITKNFQGIGPKIDAVFYSKNKYYYFFQGSNQFEYDFLLQRITKTLKSNSWFGC

SEQ ID NO: 2 - protein, 62 aa, Homo sapiens
EARNQVFLFKDDKYWLISNLRPEPNYPKSIHSFGFPNFVKKIDAAVFNPRFYRTYFFVDNQY

SEQ ID NO: 3 - protein, 21 aa, Homo sapiens
EARNQVFLFKDDKYWLISNLR

SEQ ID NO: 4 - protein, 192 aa, Mus musculus
PSTFCHQSLSFDAVTTVGEKILFFKDWFFWWKLPGSPATNITSISSIWPSIPSAIQAAYEIESRNQLFLFKDEKYWLINNLVPEPHYPRSIYSLGFSASVKKVDAAVFDPLRQKVYFFVDKHYWRYDVRQELMDPAYPKLISTHFPGIKPKIDAVLYFKRHYYIFQGAYQLEYDPLFRRVTKTLKSTSWFGC

SEQ ID NO: 5 - protein, 62 aa, Mus musculus
ESRNQLFLFKDEKYWLINNLVPEPHYPRSIYSLGFSASVKKVDAAVFDPLRQKVYFFVDKHY

SEQ ID NO: 6 - protein, 21 aa, Mus musculus
ESRNQLFLFKDEKYWLINNLV

SEQ ID NO: 7 - DNA, 578 bp, Homo sapiens
aaccagctctctgtgaccccaatttgagttttgatgctgtcactaccgtgggaaataagatctttttcttcaaagacaggttcttctggctgaaggtttctgagagaccaaagaccagtgttaatttaatttcttccttatggccaaccttgccatctggcattgaagctgcttatgaaattgaagccagaaatcaagtttttctttttaaagatgacaaatactggttaattagcaatttaagaccagagccaaattatcccaagagcatacattcttttggttttcctaactttgtgaaaaaaattgatgcagctgtttttaacccacgtttttataggacctacttctttgtagataaccagtattggaggtatgatgaaaggagacagatgatggaccctggttatcccaaactgattaccaagaacttccaaggaatcgggcctaaaattgatgcagtcttctattctaaaaacaaatactactatttcttccaaggatctaaccaatttgaatatgacttcctactccaacgtatcaccaaaacactgaaaagcaatagctggtttggttgt

SEQ ID NO: 8 - DNA, 187 bp, Homo sapiens
tgaagccagaaatcaagtttttctttttaaagatgacaaatactggttaattagcaatttaagaccagagccaaattatcccaagagcatacattcttttggttttcctaactttgtgaaaaaaattgatgcagctgtttttaacccacgtttttataggacctacttctttgtagataaccagtat

SEQ ID NO: 9 - DNA, 64 bp, Homo sapiens
tgaagccagaaatcaagtttttctttttaaagatgacaaatactggttaattagcaatttaaga

SEQ ID NO: 10 - DNA, 577 bp, Mus musculus
accatcaactttctgtcaccaaagcttgagttttgatgctgtcacaacagtgggagagaaaatccttttctttaaagactggttcttctggtggaagcttcctgggagtccagccaccaacattacttctatttcttccatatggccaagcatcccatctgctattcaagctgcttacgaaattgaaagcagaaatcaacttttcctttttaaagatgagaagtactggttaataaacaacttagtaccagagccacactatcccaggagcatatattccctgggcttctctgcatctgtgaagaaggttgatgcagctgtctttgacccacttcgccaaaaggtttatttctttgtggataaacactactggaggtatgatgtgaggcaggagctcatggaccctgcttaccccaagctgatttccacacacttcccaggaatcaagcctaaaattgatgcagtcctctatttcaaaagacactactacatcttccaaggagcctatcaattggaatatgaccccctgttccgtcgtgtcaccaaaacattgaaaagtacaagctggtttggttgt

SEQ ID NO: 11 - DNA, 186 bp, Mus musculus
gaaagcagaaatcaacttttcctttttaaagatgagaagtactggttaataaacaacttagtaccagagccacactatcccaggagcatatattccctgggcttctctgcatctgtgaagaaggttgatgcagctgtctttgacccacttcgccaaaaggtttatttctttgtggataaacactac

SEQ ID NO: 12 - DNA, 63 bp, Mus musculus
gaaagcagaaatcaacttttcctttttaaagatgagaagtactggttaataaacaacttagta

SEQ ID NO: 13 - protein, 470 aa, Homo sapiens
MKFLLILLLQATASGALPLNSSTSLEKNNVLFGERYLEKFYGLEINKLPVTKMKYSGNLMKEKIQEMQHFLGLKVTGQLDTSTLEMMHAPRCGVPDLHHFREMPGGPVWRKHYITYRINNYTPDMNREDVDYAIRKAFQVWSNVTPLKFSKINTGMADILVVFARGAHGDFHAFDGKGGILAHAFGPGSGIGGDAHFDEDEFWTTHSGGTNLFLTAVHEIGHSLGLGHSSDPKAVMFPTYKYVDINTFRLSADDIRGIQSLYGDPKENQRLPNPDNSEPALCDPNLSFDAVTTVGNKIFFFKDRFFWLKVSERPKTSVNLISSLWPTLPSGIEAAYEIEARNQVFLFKDDKYWLISNLRPEPNYPKSIHSFGFPNFVKKIDAAVFNPRFYRTYFFVDNQYWRYDERRQMMDPGYPKLITKNFQGIGPKIDAVFYSKNKYYYFFQGSNQFEYDFLLQRITKTLKSNSWFGC

SEQ ID NO: 14 - DNA, 1778 bp, Homo sapiens
tagaagtttacaatgaagtttcttctaatactgctcctgcaggccactgcttctggagctcttcccctgaacagctctacaagcctggaaaaaaataatgtgctatttggtgagagatacttagaaaaattttatggccttgagataaacaaacttccagtgacaaaaatgaaatatagtggaaacttaatgaaggaaaaaatccaagaaatgcagcacttcttgggtctgaaagtgaccgggcaactggacacatctaccctggagatgatgcacgcacctcgatgtggagtccccgatctccatcatttcagggaaatgccaggggggcccgtatggaggaaacattatatcacctacagaatcaataattacacacctgacatgaaccgtgaggatgttgactacgcaatccggaaagctttccaagtatggagtaatgttacccccttgaaattcagcaagattaacacaggcatggctgacattttggtggtttttgcccgtggagctcatggagacttccatgcttttgatggcaaaggtggaatcctagcccatgcttttggacctggatctggcattggaggggatgcacatttcgatgaggacgaattctggactacacattcaggaggcacaaacttgttcctcactgctgttcacgagattggccattccttaggtcttggccattctagtgatccaaaggctgtaatgttccccacctacaaatatgtcgacatcaacacatttcgcctctctgctgatgacatacgtggcattcagtccctgtatggagacccaaaagagaaccaacgcttgccaaatcctgacaattcagaaccagctctctgtgaccccaatttgagttttgatgctgtcactaccgtgggaaataagatctttttcttcaaagacaggttcttctggctgaaggtttctgagagaccaaagaccagtgttaatttaatttcttccttatggccaaccttgccatctggcattgaagctgcttatgaaattgaagccagaaatcaagtttttctttttaaagatgacaaatactggttaattagcaatttaagaccagagccaaattatcccaagagcatacattcttttggttttcctaactttgtgaaaaaaattgatgcagctgtttttaacccacgtttttataggacctacttctttgtagataaccagtattggaggtatgatgaaaggagacagatgatggaccctggttatcccaaactgattaccaagaacttccaaggaatcgggcctaaaattgatgcagtcttctattctaaaaacaaatactactatttcttccaaggatctaaccaatttgaatatgacttcctactccaacgtatcaccaaaacactgaaaagcaatagctggtttggttgttagaaatggtgtaattaatggtttttgttagttcacttcagcttaataagtatttattgcatatttgctatgtcctcagtgtaccactacttagagatatgtatcataaaaataaaatctgtaaaccataggtaatgattatataaaatacataatatttttcaattttgaaaactctaattgtccattcttgcttgactctactattaagtttgaaaatagttaccttcaaagcaagataattctatttgaagcatgctctgtaagttgcttcctaacatccttggactgagaaattatacttacttctggcataactaaaattaagtatatatattttggctcaaataaaattg

SEQ ID NO: 15 - protein, 462 aa, Mus musculus
MKFLMMIVFLQVSACGAAPMNDSEFAEWYLSRFYDYGKDRIPMTKTKTNRNFLKEKLQEMQQFFGLEATGQLDNSTLAIMHIPRCGVPDVQHLRAVPQRSRWMKRYLTYRIYNYTPDMKREDVDYIFQKAFQVWSDVTPLRFRKLHKDEADIMILFAFGAHGDFNYFDGKGGTLAHVFYPGPGIQGDAHFDEAETWTKSFQGTNLFLVAVHELGHSLGLQHSNNPKSIMYPTYRYLNPSTFRLSADDIRNIQSLYGAPVKPPSLTKPSSPPSTFCHQSLSFDAVTTVGEKILFFKDWFFWWKLPGSPATNITSISSIWPSIPSAIQAAYEIESRNQLFLFKDEKYWLINNLVPEPHYPRSIYSLGFSASVKKVDAAVFDPLRQKVYFFVDKHYWRYDVRQELMDPAYPKLISTHFPGIKPKIDAVLYFKRHYYIFQGAYQLEYDPLFRRVTKTLKSTSWFGC

SEQ ID NO: 16 - DNA, 1790 bp, Mus musculus
atgaaatttctcatgatgattgtgttcttacaggtatctgcctgtggggctgctcccatgaatgacagtgaatttgctgaatggtacttgtcaagattttatgattatggaaaggacagaattccaatgacaaaaacaaaaaccaatagaaacttcctaaaagaaaaactccaggaaatgcagcagttctttgggctagaagcaactgggcaactggacaactcaactctggcaataatgcacatccctcgatgtggagtgcccgatgtacagcatcttagagcagtgccccagaggtcaagatggatgaagcggtacctcacttacaggatctataattacactccggacatgaagcgtgaggatgtagactacatatttcagaaagctttccaagtctggagtgatgtgactcctctaagattcagaaagcttcataaagatgaggctgacattatgatactttttgcatttggagctcacggagacttcaactattttgatggcaaaggtggtacactagcccatgttttttatcctggacctggtattcaaggagatgcacattttgatgaggcagaaacgtggactaaaagttttcaaggcacaaacctcttccttgttgctgttcatgaacttggccattccttggggctgcagcattccaataatccaaagtcaataatgtaccccacctacagataccttaaccccagcacatttcgcctctctgctgatgacatacgtaacattcagtccctctatggagccccagtgaaacccccatccttgacaaaacctagcagtccaccatcaactttctgtcaccaaagcttgagttttgatgctgtcacaacagtgggagagaaaatccttttctttaaagactggttcttctggtggaagcttcctgggagtccagccaccaacattacttctatttcttccatatggccaagcatcccatctgctattcaagctgcttacgaaattgaaagcagaaatcaacttttcctttttaaagatgagaagtactggttaataaacaacttagtaccagagccacactatcccaggagcatatattccctgggcttctctgcatctgtgaagaaggttgatgcagctgtctttgacccacttcgccaaaaggtttatttctttgtggataaacactactggaggtatgatgtgaggcaggagctcatggaccctgcttaccccaagctgatttccacacacttcccaggaatcaagcctaaaattgatgcagtcctctatttcaaaagacactactacatcttccaaggagcctatcaattggaatatgaccccctgttccgtcgtgtcaccaaaacattgaaaagtacaagctggtttggttgttaggaagaatgtagtgaagggtgcttgctggtttttcagttttataagtatatttattacatattcactctatgctcagggtgtaactatgtggcaataatgtaacaggaaataaggggaggtgtacaggtcacacacacatagttacacagaaaagtgcttttacaaaattaacctcttttaggaacttttttcacttcattctattcttaattttgaaagtgcatggttcagaggccaactggtttatctgtaagttgttttctaacaaccttcaagtagaatattagaattagaattactctcttgtctttactgaaatgtaacatgttttgttttctttaaataattgaaagaaagtgaaaaaaaaaaaaaaaaaaaaaaaaaaacggaattcccgggga

SEQ ID NO: 17 - protein, 465 aa, Rattus norvegicus
MKFLLVLVLLVSLQVSACGAAPMNESEFAEWYLSRFFDYQGDRIPMTKTKTNRNLLEEKLQEMQQFFGLEVTGQLDTSTLKIMHTSRCGVPDVQHLRAVPQRSRWMKRYLTYRIYNYTPDMKRADVDYIFQKAFQVWSDVTPLRFRKIHKGEADITILFAFGDHGDFYDFDGKGGTLAHAFYPGPGIQGDAHFDEAETWTKSFQGTNLFLVAVHELGHSLGLRHSNNPKSIMYPTYRYLHPNTFRLSADDIHSIQSLYGAPVKNPSLTNPGSPPSTVCHQSLSFDAVTTVGDKIFFFKDWFFWWRLPGSPATNITSISSMWPTIPSGIQAAYEIGGRNQLFLFKDEKYWLINNLVPEPHYPRSIHSLGFPASVKKIDAAVFDPLRQKVYFFVDKQYWRYDVRQELMDAAYPKLISTHFPGIRPKIDAVLYFKRHYYIFQGAYQLEYDPLLDRVTKTLSSTSWFGC

SEQ ID NO: 18 - protein, 192 aa, Rattus norvegicus
PSTVCHQSLSFDAVTTVGDKIFFFKDWFFWWRLPGSPATNITSISSMWPTIPSGIQAAYEIGGRNQLFLFKDEKYWLINNLVPEPHYPRSIHSLGFPASVKKIDAAVFDPLRQKVYFFVDKQYWRYDVRQELMDAAYPKLISTHFPGIRPKIDAVLYFKRHYYIFQGAYQLEYDPLLDRVTKTLSSTSWFGC

SEQ ID NO: 19 - protein, 62 aa, Rattus norvegicus
GGRNQLFLFKDEKYWLINNLVPEPHYPRSIHSLGFPASVKKIDAAVFDPLRQKVYFFVDKQY

SEQ ID NO: 20 - protein, 21 aa, Rattus norvegicus
GGRNQLFLFKDEKYWLINNLV

SEQ ID NO: 21 - protein, 464 aa, Oryctolagus cuniculus
MKFLLLILTLWVTSSGADPLKENDMLFAENYLENFYGLKVERIPMTKMKTNRNFIEEKVQEMQQFLGLNVTGQLDTSTLEMMHKPRCGVPDVYHFKTMPGRPVWRKHYITYRIKNYTPDMKREDVEYAIQKAFQVWSDVTPLKFRKITTGKADIMILFASGAHGDYGAFDGRGGVIAHAFGPGPGIGGDTHFDEDEIWSKSYKGTNLFLVAVHELGHALGLDHSNDPKAIMFPTYGYIDLNTFHLSADDIRGIQSLYGGPEQHQPMPKPDNPEPTACDHNLKFDAVTTVGNKIFFFKDSFFWWKIPKSSTTSVRLISSLWPTLPSGIEAAYEIGDRHQVFLFKGDKFWLISHLRLQPNYPKSIHSLGFPDFVKKIDAAVFNPSLRKTYFFVDNLYWRYDERREVMDAGYPKLITKHFPGIGPKIDAVFYFQRYYYFFQGPNQLEYDTFSSRVTKKLKSNSWFDC

SEQ ID NO: 22 - protein, 26 aa, Homo sapiens
EARNQVFLFKDDKYWLISNLRPEPNY

SEQ ID NO: 23 - protein, 31 aa, Homo sapiens
EARNQVFLFKDDKYWLISNLRPEPNYPDSIH

SEQ ID NO: 24 - protein, 26 aa, Homo sapiens
AAYEIEARNQVFLFKDDKYWLISNLR

SEQ ID NO: 25 - protein, 33 aa, Homo sapiens
TLPSGIEAAYEIEARNQVFLFKDDKYWLISNLR

SEQ ID NO: 26 - protein, 31 aa, Homo sapiens
AAYEIEARNQVFLFKDDKYWLISNLRPEPNY

SEQ ID NO: 27 - protein, 38 aa, Homo sapiens
TLPSGIEAAYEIEARNQVFLFKDDKYWLISNLRPEPNY

SEQ ID NO: 28 - protein, 26 aa, Mus musculus
ESRNQLFLFKDEKYWLINNLVPEPHY

SEQ ID NO: 29 - protein, 29 aa, Mus musculus
ESRNQLFLFKDEKYWLINNLVPEPHYPRS

SEQ ID NO: 30 - protein, 26 aa, Mus musculus
AAYEIESRNQLFLFKDEKYWLINNLV

SEQ ID NO: 31 - protein, 33 aa, Mus musculus
SIPSAIQAAYEIESRNQLFLFKDEKYWLINNLV

SEQ ID NO: 32 - protein, 31 aa, Mus musculus
AAYEIESRNQLFLFKDEKYWLINNLVPEPHY

SEQ ID NO: 33 - protein, 38 aa, Mus musculus
SIPSAIQAAYEIESRNQLFLFKDEKYWLINNLVPEPHY

SEQ ID NO: 34 - DNA, 28 bp, Artificial sequence
  Synthetic Oligonucleotide
ttttatggatatcagtccaccatcaact

SEQ ID NO: 35 - DNA, 31 bp, Artificial sequence
  Synthetic Oligonucleotide
ttttagaattcgaacaaccaaaccagcttgt

SEQ ID NO: 36 - protein, 20 aa, Homo sapiens
ARNQVFLFKDDKYWLISNLR

SEQ ID NO: 37 - protein, 20 aa, Mus musculus
SRNQLFLFKDEKYWLINNLV

SEQ ID NO: 38 - DNA, 61 bp, Homo sapiens
agccagaaatcaagtttttctttttaaagatgacaaatactggttaattagcaatttaaga

SEQ ID NO: 39 - DNA, 60 bp, Mus musculus
agcagaaatcaacttttcctttttaaagatgagaagtactggttaataaacaacttagta

SEQ ID NO: 40 - protein, 20 aa, Mus musculus
RSIYSLGFSASVKKVDAAVF

SEQ ID NO: 41 - protein, 20 aa, Mus musculus
SRDLMFIFRGRKFWALNGYD

SEQ ID NO: 42 - protein, 22 aa, Homo sapiens
EARNQVFLFKDDKYWLISNLRP

SEQ ID NO: 43 - protein, 22 aa, Mus musculus
ESRNQLFLFKDEKYWLINNLVP

SEQ ID NO: 44 - DNA, 66 bp, Homo sapiens
gaagccagaaatcaagtttttctttttaaagatgacaaatactggttaattagcaatttaagacca

SEQ ID NO: 45 - DNA, 66 bp, Mus musculus
gaaagcagaaatcaacttttcctttttaaagatgagaagtactggttaataaacaacttagtacca

SEQ ID NO: 46 - protein, 19 aa, Oryctolagus cuniculus
DRHQVFLFKGDKFWLISHL

SEQ ID NO: 47 - protein, 19 aa, Rattus norvegicus
GRNQLFLFKDEKYWLINNL

SEQ ID NO: 48 - protein, 19 aa, Mus musculus
SRNQLFLFKDEKYWLINNL

SEQ ID NO: 49 - protein, 19 aa, Homo sapiens
ARNQVFLFKDDKYWLISNL

SEQ ID NO: 50 - protein, 18 aa, Mus musculus
SRDLMFIFRGRKFWALNG

SEQ ID NO: 51 - protein, 18 aa, Mus musculus
DRDLVFLFKGRQYWALSG

SEQ ID NO: 52 - protein, 18 aa, Mus musculus
IFKGSQFWAVRGNEVQAG

SEQ ID NO: 53 - protein, 17 aa, Mus musculus
GALHFFKDGWYWKFLNH

SEQ ID NO: 54 - protein, 18 aa, Mus musculus
FAGNEYWVYSASTLERGY

SEQ ID NO: 55 - protein, 20 aa, Homo sapiens
ARNQVFLFKDDKYWLISSLR